SEQUENCE LISTING (13 sequences)

SEQ ID NO: 1 - DNA, 3888 bp, Clostridium botulinum
  source 1..3888: mol_type = genomic DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - protein, 1315 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 4 - protein, 1327 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIIELGGGGSELSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 5 - protein, 1314 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 6 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 7 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 8 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 9 - protein, 10 aa, synthetic construct
TKSLDKGYNK

SEQ ID NO: 10 - protein, 8 aa, synthetic construct
SLDKGYNK

SEQ ID NO: 11 - protein, 437 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSK

SEQ ID NO: 12 - protein, 439 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTK

SEQ ID NO: 13 - protein, 856 aa, synthetic construct
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

(19) United States
(12) Patent Application Publication
(10) Pub. No.: US 2025/0213662 A1
(43) Pub. Date: Jul. 3, 2025

(54) BONT/A FOR USE IN TREATING A FACIAL DYSTONIA

(71) Applicant: IPSEN BIOPHARM LIMITED, Wrexham (GB)

(72) Inventors: Laurent Pons, Wrexham (GB); Nicolae Grigore, v (GB); Fatima Afzal Chowdhry, Wrexham (GB)

(21) Appl. No.: 18/851,469

(22) PCT Filed: Mar. 23, 2023
(86) PCT No.: PCT/GB2023/050746

§ 371 Date: Sep. 26, 2024

(30) Foreign Application Priority Data
Apr. 29, 2022 (GB) 2206361.4

(51) Int. Cl. A61K 38/48; C12N 9/52

(52) U.S. Cl. CPC A61K 38/4893; C12N 9/52; C12Y 304/24069

(57) ABSTRACT

The present invention is directed to a modified botulinum neurotoxin A (BoNT/A) for use in treating a facial dystonia, including blepharospasm and hemifacial spasm, wherein the unit dose of the modified BoNT/A is at least 240 pg of modified BoNT/A, wherein the modified BoNT/A is administered by intramuscular injection to an affected muscle of a subject, wherein the total dose administered during the treatment is greater than 24,000 pg and up to 82,500 pg (e.g. up to 75,000 pg) of the modified BoNT/A, and wherein the modified BoNT/A comprises a BoNT/A light-chain and translocation domain (HN), and a BoNT/B receptor binding domain (HC domain).

### FIELD OF THE INVENTION

The present invention relates to treatment of a disorder affecting an eyelid muscle of a subject.

### BACKGROUND

Disorders affecting an eyelid muscle can negatively-impact the life of patients suffering therefrom. Among those disorders, blepharospasm and facial spasm (e.g. hemifacial spasm) are particularly unpleasant.

Blepharospasm is characterized primarily by abnormal contractions of the orbicularis oculi muscles. More specifically, blepharospasm can manifest as an uncontrollable excessive blinking and spasming of one or both eyes that is further characterized by uncontrollable eyelid closure of durations longer than the typical blink reflex. Blepharospasm symptoms can be recurrent and may last for a few hours or days at a time and, in some cases, the symptoms (e.g. twitching) may be chronic and persistent, causing life-long challenges for subjects suffering from the condition. Other symptoms may include twitching that can radiate into the nose, face and neck, dryness of the eyes, and sensitivity to the sun and bright lights.

The cause of blepharospasm is poorly understood. It has been suggested that blepharospasm can be induced by certain drugs such as, for example, drugs to treat Parkinson's disease, estrogen-replacement therapy, or acute withdrawal from benzodiazepines. Blepharospasm may also be associated with brain disorders (e.g. including neurodegenerative conditions, abnormal functioning of the brain's basal ganglia, and multiple sclerosis), brain damage, or head injuries (e.g. concussion).

Hemifacial spasm is a movement disorder that is characterized by involuntarily tonic—clonic contractions of the mimetic muscles on one side of the face. While bilateral cases are sometimes seen, they are extremely rare. Affected muscles are those innervated by the facial nerve (cranial nerve VII). Initially symptoms of the disorder are typically located to the orbicularis oculi muscle and may spread to include other muscles of facial expression. Hemifacial spasm (HFS) takes two forms: typical HFS and atypical HFS. In the typical form, the twitching/spasm typically begins in the lower eyelid in orbicularis oculi muscle. As time progresses, it spreads to the whole lid, then to the orbicularis oris muscle around the lips, and buccinator muscle in the cheekbone area. In atypical HFS, twitching/spasm typically begins in orbicularis oris muscle around the lips, and buccinator muscle in the cheekbone area in the lower face, then progresses up to the orbicularis oculi muscle in the eyelid over time. The most common form is the typical form, and atypical form is only seen in about 2-3% of patients with hemifacial spasm.

Drug therapy for disorders affecting an eyelid muscle of a subject has proven generally unpredictable and short-termed. Anticholinergics, tranquillizing drugs and botulinum neurotoxins (e.g. Dysport®, Botox® or Xeomin®) are the most commonly used therapeutic options. However, these treatment options are not optimal and are associated with serious side effects, including toxicity and unwanted paralysis of facial muscles. In some cases, invasive surgical procedure may be envisaged for patients who do not respond well to medication or botulinum neurotoxin injection. Thus, new and effective therapies for the treatment of blepharospasm are constantly being tested or sought after.

In more detail, botulinum neurotoxin A (BoNT/A) selectively inhibits the release of acetylcholine from the presynaptic nerve terminals and thus blocks cholinergic transmission at the neuromuscular junction inducing a reduction in the muscle contraction and muscle tone, causing the injected muscles to relax. However, the duration of action of the currently available BoNT/A products is about 12 to 14 weeks, which is when the new nerve endings sprout allowing the nerve function to return to normal, and the original symptoms reappear. Consequently, for the effect to be maintained, injections need to be repeated periodically. Thus, the frequency of BoNT/A injections is an important consideration for the treatment of disorders affecting an eyelid muscle of a subject (e.g. blepharospasm and/or hemifacial spasm), considering the potential chronicity of the conditions and long-term nature of the treatment required. Indeed, this has an impact on the direct and indirect health costs involved for the patients and caregivers, the logistics for injections within the hospitals/clinics, and, most importantly, the quality of life of patients.

Dysport® is approved for the treatment of blepharospasm and hemifacial spasm with a maximum total dose per treatment session of 120 Units per eye. A clinician is required to administer Dysport® to an eyelid muscle of the subject up to the upper threshold of 120 Units total per eye per treatment session (i.e. 240 Units when treating both eyes). The clinician is forced to make difficult choices during treatment of a patient. In other words, in conventional treatment regimens, a clinician must find a balance between the relatively low total amount of BoNT/A that can be administered (necessitated by the highly toxic nature of BoNT/A) and the effective amount at a plurality of different muscles and/or sites thereof. Hence, certain muscles may be neglected while others receive a suboptimal amount of BoNT/A, resulting in suboptimal therapy.

Moreover, the conventional treatment regimens for such disorders are complicated and result in clinicians under-dosing in an effort to avoid toxicity to the patient. There is thus a need for a convenient, safe, and effective single dose unit and a corresponding guide to the number of units that can be administered to an eyelid muscle (e.g. including the number of injection sites per muscle) in a treatment session without resultant patient toxicity.

In conclusion, there is a need for an improved treatment for a disorder affecting an eyelid muscle of a subject (e.g. blepharospasm and/or hemifacial spasm) that would allow an individualised patient-centric approach to tailor the treatment according to the targeted clinical pattern permitting different combinations of muscles and/or sites thereof to be injected depending on the distribution, extent and severity of the disorder, while avoiding toxicity and providing a longer-lasting treatment (resulting in less frequent administration).

The present invention overcomes one or more of the above-mentioned problems.

### SUMMARY OF THE INVENTION

The present inventors have surprisingly found that a modified BoNT/A finds particular utility in treating a disorder affecting an eyelid muscle of a subject (e.g. blepharospasm and/or hemifacial spasm). The modified BoNT/A may comprise a BoNT/A light-chain and translocation domain and a BoNT/B receptor binding domain (H_{C} domain), which results in a modified BoNT/A that exhibits increased retention at (reduced diffusion away from) a site of administration and/or increased duration of action (e.g. 6-9 months).

Advantageously, modified BoNT/A has a safety profile that is improved when compared to unmodified BoNT/A (e.g. Dysport®). This improved safety profile may be expressed by the high Safety Ratio described herein for the modified BoNT/A.

Based on the pre-clinical data herein it has been shown that a higher total amount of modified BoNT/A may be administered to a subject while achieving a similar safety profile to unmodified BoNT/A (e.g. Dysport®) while at such high doses. Thus, more modified BoNT/A may be injected and/or may be injected at a greater number of muscles and/or sites thereof in the treatment of a disorder affecting an eyelid muscle of a subject (e.g. blepharospasm and/or hemifacial spasm) before reaching the maximum total dose. This is a significant and advantageous finding, and yields an improved treatment of such disorders while providing clinicians with a greater range of treatment options. The treatment may be improved in that it provides for longer-lasting treatment (resulting in less frequent administration) and/or is capable of being tailored for the subject and/or results in an improved quality of life of a subject when compared to treatment with unmodified BoNT/A (e.g. Dysport®). Hence, the treatment of the invention is improved compared to conventional treatment regimens.

Moreover, the present invention provides a convenient, safe, and effective single unit dose as well as a total (maximum) dosage that can be safely administered in a single treatment. The present invention also provides a corresponding guide to the number of times at which said unit dose can be administered to a muscle (e.g. including the number of injection sites per muscle) without resultant patient toxicity. Treatment of a disorder affecting an eyelid muscle of a subject (e.g. blepharospasm and/or hemifacial spasm) in accordance with the present invention is thus much less complicated for the clinician and helps avoid under-dosing and/or over-dosing. Furthermore, treatment according to the invention is much more satisfactory to the patient, as it is better tailored to the patient's needs, when compared to conventional treatments.

### DETAILED DESCRIPTION

Broad aspects of the invention provide:
a modified botulinum neurotoxin A (BoNT/A) for use in a method of treating blepharospasm in a subject, wherein the modified BoNT/A is administered by intramuscular injection at a plurality of sites of the face of the subject
a method of treating blepharospasm in a subject, wherein method comprises administering a modified BoNT/A by intramuscular injection at a plurality of sites of the face of the subject
use of a modified botulinum neurotoxin A (BoNT/A) in the manufacture of a medicament for treating blepharospasm in a subject, wherein the modified BoNT/A is administered by intramuscular injection at a plurality of sites of the face of the subject;

wherein the modified BoNT/A is administered as a unit dose comprising at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

Further broad aspects of the invention provide:
a modified botulinum neurotoxin A (BoNT/A) for use in a method of treating typical hemifacial spasm, wherein the modified BoNT/A is administered by intramuscular injection at a plurality of sites of the face of the subject;
a method of treating typical hemifacial spasm, the method comprising administering a modified BoNT/A by intramuscular injection at a plurality of sites of the face of the subject;
use of a modified botulinum neurotoxin A (BoNT/A) in the manufacture of a medicament for treating typical hemifacial spasm, wherein the modified BoNT/A is administered by intramuscular injection at a plurality of sites of the face of the subject;
wherein the modified BoNT/A is administered as a unit dose comprising at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

Further broad aspects of the invention provide:
a modified botulinum neurotoxin A (BoNT/A) for use in a method of treating atypical hemifacial spasm, wherein the modified BoNT/A is administered by intramuscular injection at a plurality of sites of the face of the subject;
a method of treating atypical hemifacial spasm, the method comprising administering a modified BoNT/A by intramuscular injection at a plurality of sites of the face of the subject;
use of a modified botulinum neurotoxin A (BoNT/A) in the manufacture of a medicament for treating atypical hemifacial spasm, wherein the modified BoNT/A is administered by intramuscular injection at a plurality of sites of the face of the subject;
wherein the modified BoNT/A is administered as a unit dose comprising at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

One aspect provides a modified botulinum neurotoxin A (BoNT/A) for use in a method of treating blepharospasm in a subject, wherein the modified BoNT/A is administered by intramuscular injection at a plurality of sites of the face of the subject, the method comprising:
a) administering a unit dose of the modified BoNT/A per injection site at up to six different injection sites of the upper orbicularis oculi muscle proximal to a first eye of the subject, wherein said up to six different injection sites are selected from:
   i) the medial upper orbicularis oculi muscle (e.g. at a preseptal portion or an orbital portion of said medial upper orbicularis oculi muscle, preferably a preseptal portion);
   ii) the superior orbital orbicularis oculi muscle;
   iii) the lateral upper orbicularis oculi muscle (e.g. at a preseptal portion or an orbital portion of said lateral upper orbicularis oculi muscle, preferably a preseptal portion);
   iv) the outer orbital orbicularis oculi muscle;
   v) the medial upper pretarsal orbital orbicularis oculi muscle; and
   vi) the lateral upper pretarsal orbital orbicularis oculi muscle; and/or
b) administering a unit dose of the modified BoNT/A per injection site at up to two different injection sites of the lower orbicularis oculi muscle proximal to the first eye of the subject, wherein said up to two different injection sites are selected from:
   i) the medial lower orbicularis oculi muscle (e.g. at a preseptal portion or an orbital portion of said medial lower orbicularis oculi muscle, preferably a preseptal portion); and
   ii) the lateral lower orbicularis oculi muscle (e.g. at a preseptal portion or an orbital portion of said lateral lower orbicularis oculi muscle, preferably a preseptal portion); and/or
c) administering a unit dose of the modified BoNT/A per injection site at up to two different injection sites selected from:
   i) two different injection sites of the corrugator proximal to the first eye of the subject; and
   ii) one site on the procerus proximal to the first eye of the subject;
   wherein the unit dose of the modified BoNT/A is at least 240 pg (preferably 240 pg to 8,000 pg) of modified BoNT/A,
   wherein the total dose administered during the treatment is greater than 24,000 pg and up to 82,500 pg (e.g. up to 75,000 pg) of the modified BoNT/A, and
   wherein the modified BoNT/A comprises a BoNT/A light-chain and translocation domain (H_{N}), and a BoNT/B receptor binding domain (H_{C} domain).

One aspect provides a method of treating blepharospasm in a subject, the method comprising administering a modified botulinum neurotoxin A (BoNT/A) by intramuscular injection at a plurality of sites of the face of the subject, the method comprising:
a) administering a unit dose of the modified BoNT/A per injection site at up to six different injection sites of the upper orbicularis oculi muscle proximal to a first eye of the subject, wherein said up to six different injection sites are selected from:
   i) the medial upper orbicularis oculi muscle (e.g. at a preseptal portion or an orbital portion of said medial upper orbicularis oculi muscle, preferably a preseptal portion);
   ii) the superior orbital orbicularis oculi muscle;
   iii) the lateral upper orbicularis oculi muscle (e.g. at a preseptal portion or an orbital portion of said lateral upper orbicularis oculi muscle, preferably a preseptal portion);
   iv) the outer orbital orbicularis oculi muscle;
   v) the medial upper pretarsal orbital orbicularis oculi muscle; and
   vi) the lateral upper pretarsal orbital orbicularis oculi muscle; and/or
b) administering a unit dose of the modified BoNT/A per injection site at up to two different injection sites of the lower orbicularis oculi muscle proximal to the first eye of the subject, wherein said up to two different injection sites are selected from:
   i) the medial lower orbicularis oculi muscle (e.g. at a preseptal portion or an orbital portion of said medial lower orbicularis oculi muscle, preferably a preseptal portion); and
   ii) the lateral lower orbicularis oculi muscle (e.g. at a preseptal portion or an orbital portion of said lateral lower orbicularis oculi muscle, preferably a preseptal portion); and/or
c) administering a unit dose of the modified BoNT/A per injection site at up to two different injection sites selected from:
   i) two different injection sites of the corrugator proximal to the first eye of the subject; and
   ii) one site on the procerus proximal to the first eye of the subject;
   wherein the unit dose of the modified BoNT/A is at least 240 pg (preferably 240 pg to 8,000 pg) of modified BoNT/A,
   wherein the total dose administered during the treatment is greater than 24,000 pg and up to 82,500 pg (e.g. up to 75,000 pg) of the modified BoNT/A, and
   wherein the modified BoNT/A comprises a BoNT/A light-chain and translocation domain (H_{N}), and a BoNT/B receptor binding domain (H_{C} domain).

One aspect provides use of a modified botulinum neurotoxin A (BoNT/A) in the manufacture of a medicament for treating typical hemifacial spasm, wherein the modified BoNT/A is administered by intramuscular injection at a plurality of sites of the face of the subject, the treatment comprising:
(a) administering a unit dose of the modified BoNT/A per injection site at up to six different injection sites of the upper orbicularis oculi muscle proximal to a first eye of the subject, wherein said up to six different injection sites are selected from:
   (i) the medial upper orbicularis oculi muscle (e.g. at a preseptal portion or an orbital portion medial upper orbicularis oculi muscle, preferably a preseptal portion);
   (ii) the superior orbital orbicularis oculi muscle;
   (iii) the lateral upper orbicularis oculi muscle (e.g. at a preseptal portion or an orbital portion of said lateral upper orbicularis oculi muscle, preferably a preseptal portion);
   (iv) the outer orbital orbicularis oculi muscle;
   (v) the medial upper pretarsal orbital orbicularis oculi muscle; and
   (vi) the lateral upper pretarsal orbital orbicularis oculi muscle; and/or
(b) administering a unit dose of the modified BoNT/A per injection site at up to two different injection sites of the lower orbicularis oculi muscle proximal to the first eye of the subject, wherein said up to two different injection sites are selected from:
   (i) the medial lower orbicularis oculi muscle (e.g. at a preseptal portion or an orbital portion of said medial lower orbicularis oculi muscle, preferably a preseptal portion); and
   (ii) the lateral lower orbicularis oculi muscle (e.g. at a preseptal portion or an orbital portion of said lateral lower orbicularis oculi muscle, preferably a preseptal portion); and/or
(c) administering a unit dose of the modified BoNT/A per injection site at up to two different injection sites selected from:
   (i) two different injection sites of the corrugator proximal to the first eye of the subject; and
   (ii) one site on the procerus proximal to the first eye of the subject;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a BoNT/A light-chain and translocation domain (H_{N}), and a BoNT/B receptor binding domain (H_{C} domain).

Throughout this disclosure, the injection site “outer orbital orbicularis oculi muscle” is referred to in the context of the injection sites of “the upper orbicularis oculi muscle”, e.g. due to proximity of site “outer orbital orbicularis oculi muscle” to the upper eyelid relative to the lower eyelid. That being said, in any aspect of embodiment described herein (whether for the treatment of blepharospasm, typical hemifacial spasm, or atypical hemifacial spasm) the following term quoted under (1) may be used synonymously with the following term quoted under (2):
(1) “administering a unit dose of the modified BoNT/A per injection site at up to six different injection sites of the upper orbicularis oculi muscle proximal to a first eye (and/or second) of the subject, wherein said up to six different injection sites are selected from:
   (iii) the medial upper orbicularis oculi muscle (e.g. at a preseptal portion or an orbital portion medial upper orbicularis oculi muscle, preferably a preseptal portion);
   (iv) the superior orbital orbicularis oculi muscle;
   (v) the lateral upper orbicularis oculi muscle (e.g. at a preseptal portion or an orbital portion of said lateral upper orbicularis oculi muscle, preferably a preseptal portion);
   (vi) the outer orbital orbicularis oculi muscle;
   (vii) the medial upper pretarsal orbital orbicularis oculi muscle; and
   (viii) the lateral upper pretarsal orbital orbicularis oculi muscle”;
(2) “administering a unit dose of the modified BoNT/A per injection site at up to six different injection sites of the upper orbicularis oculi muscle and/or the outer orbital orbicularis oculi muscle proximal to a first (and/or second) eye of the subject, wherein said up to six different injection sites are selected from:
   (i) the medial upper orbicularis oculi muscle (e.g. at a preseptal portion or an orbital portion medial upper orbicularis oculi muscle, preferably a preseptal portion);
   (ii) the superior orbital orbicularis oculi muscle;
   (iii) the lateral upper orbicularis oculi muscle (e.g. at a preseptal portion or an orbital portion of said lateral upper orbicularis oculi muscle, preferably a preseptal portion);
   (iv) the outer orbital orbicularis oculi muscle;
   (v) the medial upper pretarsal orbital orbicularis oculi muscle; and
   (vi) the lateral upper pretarsal orbital orbicularis oculi muscle”.

The modified BoNT/A may preferably be administered by intramuscular injection at at least six different sites of the face of the subject. In other words, the modified BoNT/A may preferably be administered by intramuscular injection to at least six different sites of the face of the subject. In yet other words, the number of different injection sites that a unit dose is administered to may preferably be at least six.

The injection regimens of the invention allows a clinician to accommodate the individual pattern of involvement of the muscles in the participants' blepharospasm that guide where the injections will be placed across disclosed muscles. At the same time, the risk of reducing ptosis can be mitigated.

If the pretarsal muscles in the upper eyelid are involved, preferably a maximum of two injections may be placed in the pretarsal part of the orbicularis oculi upper eyelid. The regimen preferably avoids the sulcus of the upper eyelid due thus reducing the likelihood of developing ptosis. If the corrugator/procerus muscles are involved, preferably up to two injections may be placed between the eyebrows on each side.

A method for treating blepharospasm may further comprises:
(a) administering a unit dose of the modified BoNT/A per injection site at up to six different injection sites of the upper orbicularis oculi muscle proximal to a second eye of the subject, wherein said up to six different injection sites are selected from:
   (i) the medial upper orbicularis oculi muscle (e.g. at a preseptal portion or an orbital portion of said medial upper orbicularis oculi muscle, preferably a preseptal portion);
   (ii) the superior orbital orbicularis oculi muscle;
   (iii) the lateral upper orbicularis oculi muscle (e.g. at a preseptal portion or an orbital portion of said lateral upper orbicularis oculi muscle, preferably a preseptal portion);
   (iv) the outer orbital orbicularis oculi muscle;
   (v) the medial upper pretarsal orbital orbicularis oculi muscle; and
   (vi) the lateral upper pretarsal orbital orbicularis oculi muscle; and/or
(b) administering a unit dose of the modified BoNT/A per injection site at up to two different injection sites of the lower orbicularis oculi muscle proximal to the second eye of the subject, wherein said up to two different injection sites are selected from:
   (i) the medial lower orbicularis oculi muscle (e.g. at a preseptal portion or an orbital portion of said medial lower orbicularis oculi muscle, preferably a preseptal portion); and
   (ii) the lateral lower orbicularis oculi muscle; and/or
(c) administering a unit dose of the modified BoNT/A per injection site at up to two different injection sites selected from:
   (i) two different injection sites of the corrugator proximal to the second eye of the subject; and
   (ii) one site on the procerus proximal to the second eye of the subject.

The modified BoNT/A may preferably be administered by intramuscular injection at at least six different sites of the face of the subject. In other words, the modified BoNT/A may preferably be administered by intramuscular injection to at least six different sites of the face of the subject. In yet other words, the number of different injection sites that a unit dose is administered to may preferably be at least six.

A method for treating blepharospasm may preferably comprises:
(a) administering a unit dose of the modified BoNT/A to the lateral upper orbicularis oculi muscle (e.g. at a preseptal portion or an orbital portion of said muscle, preferably the lateral upper preseptal orbicularis oculi muscle) proximal to a first eye of the subject;
(b) administering a unit dose of the modified BoNT/A to the medial upper orbicularis oculi muscle (e.g. at a preseptal portion or an orbital portion of said muscle, preferably the medial upper preseptal orbicularis oculi muscle) proximal to the first eye of the subject; and
(c) administering a unit dose of the modified BoNT/A to the lateral lower orbicularis oculi muscle (e.g. at a preseptal portion or an orbital portion of said muscle, preferably the lateral lower orbicularis oculi muscle) proximal to the first eye of the subject.

In other words, a method for treating blepharospasm may preferably comprise administering a unit dose of the modified BoNT/A to each of:
(a) the lateral upper orbicularis oculi muscle (e.g. at a preseptal portion or an orbital portion of said muscle, preferably the lateral upper preseptal orbicularis oculi muscle) proximal to a first eye of the subject;
(b) the medial upper orbicularis oculi muscle (e.g. at a preseptal portion or an orbital portion of said muscle, preferably the medial upper preseptal orbicularis oculi muscle) proximal to the first eye of the subject; and
(c) the lateral lower orbicularis oculi muscle (e.g. at a preseptal portion or an orbital portion of said muscle, preferably the lateral lower orbicularis oculi muscle) proximal to the first eye of the subject.

One aspect provides a modified botulinum neurotoxin A (BoNT/A) for use in a method of treating blepharospasm in a subject, wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the lateral lower preseptal orbicularis oculi muscle) proximal to the first eye of the subject,
   wherein the unit dose of the modified BoNT/A is at least 240 pg (preferably 240 pg to 8,000 pg) of modified BoNT/A,
   wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
   wherein the modified BoNT/A comprises a BoNT/A light-chain and translocation domain (H_{N}), and a BoNT/B receptor binding domain (H_{C} domain).

In a related aspect, the invention provides a modified botulinum neurotoxin A (BoNT/A) for use in a method of treating blepharospasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the lateral lower preseptal orbicularis oculi muscle) proximal to the first eye of the subject,
   wherein the unit dose of the modified BoNT/A is at least 240 pg (preferably 240 pg to 8,000 pg) of modified BoNT/A,
   wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
   wherein the modified BoNT/A comprises a BoNT/A light-chain and translocation domain (H_{N}), and a BoNT/B receptor binding domain (H_{C} domain).

In a related aspect, the invention provides a method of treating blepharospasm in a subject, wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the lateral lower preseptal orbicularis oculi muscle) proximal to the first eye of the subject,
   wherein the unit dose of the modified BoNT/A is at least 240 pg (preferably 240 pg to 8,000 pg) of modified BoNT/A,
   wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
   wherein the modified BoNT/A comprises a BoNT/A light-chain and translocation domain (HN), and a BoNT/B receptor binding domain (HC domain).

In a related aspect, the invention provides a method of treating blepharospasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the lateral lower preseptal orbicularis oculi muscle) proximal to the first eye of the subject,
   wherein the unit dose of the modified BoNT/A is at least 240 pg (preferably 240 pg to 8,000 pg) of modified BoNT/A,
   wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
   wherein the modified BoNT/A comprises a BoNT/A light-chain and translocation domain (HN), and a BoNT/B receptor binding domain (HC domain).

In one aspect, the invention provides the use of a modified botulinum neurotoxin A (BoNT/A) in the manufacture of medicament for treating blepharospasm in a subject, comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the lateral lower preseptal orbicularis oculi muscle) proximal to the first eye of the subject,
   wherein the unit dose of the modified BoNT/A is at least 240 pg (preferably 240 pg to 8,000 pg) of modified BoNT/A,
   wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
   wherein the modified BoNT/A comprises a BoNT/A light-chain and translocation domain (HN), and a BoNT/B receptor binding domain (HC domain).

In one aspect, the invention provides the use of a modified botulinum neurotoxin A (BoNT/A) in the manufacture of medicament for treating blepharospasm in a subject for a longer duration than that treated by an unmodified BoNT/A (e.g. SEQ ID NO: 2 [such as a di-chain form of SEQ ID NO: 2]), comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the lateral lower preseptal orbicularis oculi muscle) proximal to the first eye of the subject,
   wherein the unit dose of the modified BoNT/A is at least 240 pg (preferably 240 pg to 8,000 pg) of modified BoNT/A,
   wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
   wherein the modified BoNT/A comprises a BoNT/A light-chain and translocation domain (HN), and a BoNT/B receptor binding domain (HC domain).

One aspect provides a modified botulinum neurotoxin A (BoNT/A) for use in a method of treating typical hemifacial spasm, wherein the modified BoNT/A is administered by intramuscular injection at a plurality of sites of the face of the subject, the method comprising:
(a) administering a unit dose of the modified BoNT/A per injection site at up to six different injection sites of the upper orbicularis oculi muscle proximal to a first eye of the subject, wherein said up to six different injection sites are selected from:
   (i) the medial upper orbicularis oculi muscle (e.g. at a preseptal portion or an orbital portion of said muscle, preferably a preseptal portion);
   (ii) the superior orbital orbicularis oculi muscle;
   (iii) the lateral upper orbicularis oculi muscle (e.g. at a preseptal portion or an orbital portion of said muscle, preferably a preseptal portion);
   (iv) the outer orbital orbicularis oculi muscle;
   (v) the medial upper pretarsal orbital orbicularis oculi muscle; and
   (vi) the lateral upper pretarsal orbital orbicularis oculi muscle; and/or
(b) administering a unit dose of the modified BoNT/A per injection site at up to two different injection sites of the lower orbicularis oculi muscle proximal to the first eye of the subject, wherein said up to two different injection sites are selected from:
   (i) the medial lower orbicularis oculi muscle (e.g. at a preseptal portion or an orbital portion of said muscle, preferably a preseptal portion); and
   (ii) the lateral lower orbicularis oculi muscle (e.g. at a preseptal portion or an orbital portion of said muscle, preferably a preseptal portion); and/or
(c) administering a unit dose of the modified BoNT/A per injection site at up to two different injection sites selected from:
   (i) two different injection sites of the corrugator proximal to the first eye of the subject; and
   (ii) one site on the procerus proximal to the first eye of the subject; and/or
(d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
      wherein the unit dose of the modified BoNT/A is at least 240 pg (preferably 240 pg to 8,000 pg) of modified BoNT/A,
      wherein the total dose administered during the treatment is greater than 24,000 pg and up to 82,500 pg (e.g. up to 75,000 pg) of the modified BoNT/A, and
      wherein the modified BoNT/A comprises a botulinum neurotoxin A (BoNT/A) light-chain and translocation domain (H_{N}), and a BoNT/B receptor binding domain (H_{C} domain).

One aspect provides a method of treating typical hemifacial spasm, the method comprising administering a modified BoNT/A by intramuscular injection at a plurality of sites of the face of the subject, the method comprising:
(a) administering a unit dose of the modified BoNT/A per injection site at up to six different injection sites of the upper orbicularis oculi muscle proximal to a first eye of the subject, wherein said up to six different injection sites are selected from:
   (i) the medial upper orbicularis oculi muscle (e.g. at a preseptal portion or an orbital portion of said muscle, preferably a preseptal portion);
   (ii) the superior orbital orbicularis oculi muscle;
   (iii) the lateral upper orbicularis oculi muscle (e.g. at a preseptal portion or an orbital portion of said muscle, preferably a preseptal portion);
   (iv) the outer orbital orbicularis oculi muscle;
   (v) the medial upper pretarsal orbital orbicularis oculi muscle; and
   (vi) the lateral upper pretarsal orbital orbicularis oculi muscle; and/or
(b) administering a unit dose of the modified BoNT/A per injection site at up to two different injection sites of the lower orbicularis oculi muscle proximal to the first eye of the subject, wherein said up to two different injection sites are selected from:
   (i) the medial lower orbicularis oculi muscle (e.g. at a preseptal portion or an orbital portion of said muscle, preferably a preseptal portion); and
   (ii) the lateral lower orbicularis oculi muscle (e.g. at a preseptal portion or an orbital portion of said muscle, preferably a preseptal portion); and/or
(c) administering a unit dose of the modified BoNT/A per injection site at up to two different injection sites selected from:
   (i) two different injection sites of the corrugator proximal to the first eye of the subject; and
   (ii) one site on the procerus proximal to the first eye of the subject; and/or
(d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
      wherein the unit dose of the modified BoNT/A is at least 240 pg (preferably 240 pg to 8,000 pg) of modified BoNT/A,
      wherein the total dose administered during the treatment is greater than 24,000 pg and up to 82,500 pg (e.g. up to 75,000 pg) of the modified BoNT/A, and
      wherein the modified BoNT/A comprises a botulinum neurotoxin A (BoNT/A) light-chain and translocation domain (H_{N}), and a BoNT/B receptor binding domain (H_{C} domain).

One aspect provides use of a modified botulinum neurotoxin A (BoNT/A) in the manufacture of a medicament for treating typical hemifacial spasm, wherein the modified BoNT/A is administered by intramuscular injection at a plurality of sites of the face of the subject, the treatment comprising:
(a) administering a unit dose of the modified BoNT/A per injection site at up to six different injection sites of the upper orbicularis oculi muscle proximal to a first eye of the subject, wherein said up to six different injection sites are selected from:
   (i) the medial upper orbicularis oculi muscle (e.g. at a preseptal portion or an orbital portion of said muscle, preferably a preseptal portion);
   (ii) the superior orbital orbicularis oculi muscle;
   (iii) the lateral upper orbicularis oculi muscle (e.g. at a preseptal portion or an orbital portion of said muscle, preferably a preseptal portion);
   (iv) the outer orbital orbicularis oculi muscle;
   (v) the medial upper pretarsal orbital orbicularis oculi muscle; and
   (vi) the lateral upper pretarsal orbital orbicularis oculi muscle; and/or
(b) administering a unit dose of the modified BoNT/A per injection site at up to two different injection sites of the lower orbicularis oculi muscle proximal to the first eye of the subject, wherein said up to two different injection sites are selected from:
   (i) the medial lower orbicularis oculi muscle (e.g. at a preseptal portion or an orbital portion of said muscle, preferably a preseptal portion); and
   (ii) the lateral lower orbicularis oculi muscle (e.g. at a preseptal portion or an orbital portion of said muscle, preferably a preseptal portion); and/or
(c) administering a unit dose of the modified BoNT/A per injection site at up to two different injection sites selected from:
   (i) two different injection sites of the corrugator proximal to the first eye of the subject; and
   (ii) one site on the procerus proximal to the first eye of the subject; and/or
(d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
      wherein the unit dose of the modified BoNT/A is at least 240 pg (preferably 240 pg to 8,000 pg) of modified BoNT/A,
      wherein the total dose administered during the treatment is greater than 24,000 pg and up to 82,500 pg (e.g. up to 75,000 pg) of the modified BoNT/A, and
      wherein the modified BoNT/A comprises a botulinum neurotoxin A (BoNT/A) light-chain and translocation domain (H_{N}), and a BoNT/B receptor binding domain (H_{C} domain).

The modified BoNT/A may preferably be administered by intramuscular injection at at least six different sites of the face of the subject. In other words, the modified BoNT/A may preferably be administered by intramuscular injection to at least six different sites of the face of the subject. In yet other words, the number of different injection sites that a unit dose is administered to may preferably be at least six.

A method for treating typical hemifacial spasm may preferably comprise:
(a) administering a unit dose of the modified BoNT/A to the lateral upper orbicularis oculi muscle (e.g. at a preseptal portion or an orbital portion of said muscle, preferably the lateral upper preseptal orbicularis oculi muscle) proximal to a first eye of the subject;
(b) administering a unit dose of the modified BoNT/A to the medial upper orbicularis oculi muscle (e.g. at a preseptal portion or an orbital portion of said muscle, preferably the medial upper preseptal orbicularis oculi muscle) proximal to the first eye of the subject; and
(c) administering a unit dose of the modified BoNT/A to the lateral lower orbicularis oculi muscle (e.g. at a preseptal portion or an orbital portion of said muscle, preferably the lateral lower orbicularis oculi muscle) proximal to the first eye of the subject.

In other words, a method for treating typical hemifacial spasm may preferably comprise administering a unit dose of the modified BoNT/A to each of:
(a) the lateral upper orbicularis oculi muscle (e.g. at a preseptal portion or an orbital portion of said muscle, preferably the lateral upper preseptal orbicularis oculi muscle) proximal to a first eye of the subject;
(b) the medial upper orbicularis oculi muscle (e.g. at a preseptal portion or an orbital portion of said muscle, preferably the medial upper preseptal orbicularis oculi muscle) proximal to the first eye of the subject; and
(c) the lateral lower orbicularis oculi muscle (e.g. at a preseptal portion or an orbital portion of said muscle, preferably the lateral lower orbicularis oculi muscle) proximal to the first eye of the subject.

Another aspect provides a modified botulinum neurotoxin A (BoNT/A) for use in a method of treating typical hemifacial spasm, wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle (preferably the lateral lower preseptal orbicularis oculi muscle) proximal to an eye affected by hemifacial spasm; and
d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

In a related aspect, the invention provides a modified botulinum neurotoxin A (BoNT/A) for use in a method of treating typical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle proximal to an eye affected by hemifacial spasm; and
d) administering one or more unit dose of the modified BoNT/A to one or more further muscles (preferably the lateral lower preseptal orbicularis oculi muscle) affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

In a related aspect, the invention provides a method of treating typical hemifacial spasm in a subject, wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle (preferably the lateral lower preseptal orbicularis oculi muscle) proximal to an eye affected by hemifacial spasm; and
d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

In a related aspect, the invention provides a method of treating typical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle (preferably the lateral lower preseptal orbicularis oculi muscle) proximal to an eye affected by hemifacial spasm; and
d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

In one aspect, the invention provides the use of a modified botulinum neurotoxin A (BoNT/A) in the manufacture of medicament for treating typical hemifacial spasm in a subject, wherein the modified BoNT/A is administered by intramuscular injection at a plurality of sites of the face of the subject, comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle (preferably the lateral lower preseptal orbicularis oculi muscle) proximal to an eye affected by hemifacial spasm; and
d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

In one aspect, the invention provides the use of a modified botulinum neurotoxin A (BoNT/A) in the manufacture of medicament for treating typical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle (preferably the lateral lower preseptal orbicularis oculi muscle) proximal to an eye affected by hemifacial spasm; and
d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

One aspect provides a modified botulinum neurotoxin A (BoNT/A) for use in treating atypical hemifacial spasm, wherein the modified BoNT/A is administered by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus minor muscle;
   (iii) up to five unit doses (preferably one unit dose) to a frontalis muscle;
   (iv) one unit dose to a mentalis muscle;
   (v) one unit dose to a platysma muscle;
   (vi) one unit dose to a buccinator muscle;
   (vii) up to two unit doses (preferably one unit dose) to a masseter muscle;
   (viii) one unit dose to a nasalis muscle;
   (ix) one unit dose to a levator palpebrae superiori muscle; and/or
c) optionally administering one or more unit dose of the modified BoNT/A to one or more further muscles affected by said hemifacial spasm in accordance with the following dosage regimen:
   a unit dose per injection site at up to six different injection sites of the upper orbicularis oculi muscle selected from:
      (i) the medial upper orbicularis oculi muscle (e.g. at a preseptal portion or an orbital portion of said muscle, preferably a preseptal portion);
      (ii) the superior orbital orbicularis oculi muscle;
      (iii) the lateral upper orbicularis oculi muscle (e.g. at a preseptal portion or an orbital portion of said muscle, preferably a preseptal portion);
      (iv) the outer orbital orbicularis oculi muscle;
      (v) the medial upper pretarsal orbital orbicularis oculi muscle; and
      (vi) the lateral upper pretarsal orbital orbicularis oculi muscle; and/or
   a unit dose per injection site at up to two different injection sites of the lower orbicularis oculi muscle selected from:
      (i) the medial lower orbicularis oculi muscle (e.g. at a preseptal portion or an orbital portion of said muscle, preferably a preseptal portion); and
      (ii) the lateral lower orbicularis oculi muscle (e.g. at a preseptal portion or an orbital portion of said muscle, preferably a preseptal portion); and/or
   a unit dose per injection site at up to two different injection sites selected from:
      (i) two different injection sites of the corrugator; and
      (ii) one site on the procerus;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

One aspect provides a method of treating atypical hemifacial spasm, the method comprising administering a modified BoNT/A by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
   (x) one unit dose to a zygomaticus major muscle;
   (xi) one unit dose to a zygomaticus minor muscle;
   (xii) up to five unit doses (preferably one unit dose) to a frontalis muscle;
   (xiii) one unit dose to a mentalis muscle;
   (xiv) one unit dose to a platysma muscle;
   (xv) one unit dose to a buccinator muscle;
   (xvi) up to two unit doses (preferably one unit dose) to a masseter muscle;
   (xvii) one unit dose to a nasalis muscle;
   (xviii) one unit dose to a levator palpebrae superiori muscle; and/or
c) optionally administering one or more unit dose of the modified BoNT/A to one or more further muscles affected by said hemifacial spasm in accordance with the following dosage regimen:
   a unit dose per injection site at up to six different injection sites of the upper orbicularis oculi muscle selected from:
      (i) the medial upper orbicularis oculi muscle (e.g. at a preseptal portion or an orbital portion of said muscle, preferably a preseptal portion);
      (ii) the superior orbital orbicularis oculi muscle;
      (iii) the lateral upper orbicularis oculi muscle (e.g. at a preseptal portion or an orbital portion of said muscle, preferably a preseptal portion);
      (iv) the outer orbital orbicularis oculi muscle;
      (v) the medial upper pretarsal orbital orbicularis oculi muscle; and
      (vi) the lateral upper pretarsal orbital orbicularis oculi muscle; and/or a unit dose per injection site at up to two different injection sites of the lower orbicularis oculi muscle selected from:
      (i) the medial lower orbicularis oculi muscle (e.g. at a preseptal portion or an orbital portion of said muscle, preferably a preseptal portion); and
      (ii) the lateral lower orbicularis oculi muscle (e.g. at a preseptal portion or an orbital portion of said muscle, preferably a preseptal portion); and/or
   a unit dose per injection site at up to two different injection sites selected from:
      (i) two different injection sites of the corrugator; and
      (ii) one site on the procerus;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

One aspect provides use of a modified botulinum neurotoxin A (BoNT/A) in the manufacture of a medicament for treating atypical hemifacial spasm, wherein the modified BoNT/A is administered by intramuscular injection at a plurality of sites of the face of the subject, the treatment comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus minor muscle;
   (iii) up to five unit doses (preferably one unit dose) to a frontalis muscle;
   (iv) one unit dose to a mentalis muscle;
   (v) one unit dose to a platysma muscle;
   (vi) one unit dose to a buccinator muscle;
   (vii) up to two unit doses (preferably one unit dose) to a masseter muscle;
   (viii) one unit dose to a nasalis muscle;
   (ix) one unit dose to a levator palpebrae superiori muscle; and/or
c) optionally administering one or more unit dose of the modified BoNT/A to one or more further muscles affected by said hemifacial spasm in accordance with the following dosage regimen:
   a unit dose per injection site at up to six different injection sites of the upper orbicularis oculi muscle selected from:
      (i) the medial upper orbicularis oculi muscle (e.g. at a preseptal portion or an orbital portion of said muscle, preferably a preseptal portion);
      (ii) the superior orbital orbicularis oculi muscle;
      (iii) the lateral upper orbicularis oculi muscle (e.g. at a preseptal portion or an orbital portion of said muscle, preferably a preseptal portion);
      (iv) the outer orbital orbicularis oculi muscle;
      (v) the medial upper pretarsal orbital orbicularis oculi muscle; and
      (vi) the lateral upper pretarsal orbital orbicularis oculi muscle; and/or
   a unit dose per injection site at up to two different injection sites of the lower orbicularis oculi muscle selected from:
      (i) the medial lower orbicularis oculi muscle (e.g. at a preseptal portion or an orbital portion of said muscle, preferably a preseptal portion); and
      (ii) the lateral lower orbicularis oculi muscle (e.g. at a preseptal portion or an orbital portion of said muscle, preferably a preseptal portion); and/or
   a unit dose per injection site at up to two different injection sites selected from:
      (i) two different injection sites of the corrugator; and
      (ii) one site on the procerus;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

Another aspect provides a modified botulinum neurotoxin A (BoNT/A) for use in a method of treating atypical hemifacial spasm, wherein the modified BoNT/A is administered by intramuscular injection at a plurality of sites of the face of the subject, the method comprising:
a) administering a unit dose of the modified BoNT/A to an orbicularis oris muscle affected by said hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

In a related aspect, the invention provides a modified botulinum neurotoxin A (BoNT/A) for use in a method of treating atypical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to an orbicularis oris muscle affected by said hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

In a related aspect, the invention provides a method of treating atypical hemifacial spasm in a subject, wherein the modified BoNT/A is administered by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

In a related aspect, the invention provides a method of treating atypical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

In one aspect, the invention provides the use of a modified botulinum neurotoxin A (BoNT/A) in the manufacture of medicament for treating atypical hemifacial spasm in a subject, wherein the modified BoNT/A is administered by intramuscular injection at a plurality of sites of the face of the subject, comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

In one aspect, the invention provides the use of a modified botulinum neurotoxin A (BoNT/A) in the manufacture of medicament for treating atypical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg (e.g. preferably greater than 24,100 pg; more preferably greater than 35,000 pg) and up to 82,500 pg (e.g. up to 75,000 pg) of the modified BoNT/A, and
wherein the modified BoNT/A comprises a BoNT/A light-chain and translocation domain (HN), and a BoNT/B receptor binding domain (HC domain).

Throughout this disclosure, reference to a total dose administered during the treatment of greater than 24,000 pg of the modified BoNT/A may mean that total dose administered during the treatment is greater than 24,100 pg of the modified BoNT/A. More preferably, reference to a total dose administered during the treatment of greater than 24,000 pg of the modified BoNT/A may mean that total dose administered during the treatment is greater than 35,000 pg of the modified BoNT/A.

The term “typical hemifacial spasm” may be used interchangeably with the term “hemifacial spasm” throughout this disclosure.

The term “treating blepharospasm of a subject for a longer duration than that treated by an unmodified BoNT/A” may mean that one or more symptoms of said disorder of the subject are reduced for a longer time period (e.g. 6-9 months) following administration of a modified BoNT/A of the invention, when compared to administration of an unmodified BoNT/A. Said reduction may be determined by comparison to an equivalent control subject exhibiting equivalent symptoms that has been treated with an unmodified BoNT/A. At a time period where the severity of one or more symptoms of the control subject are substantially the same (e.g. the same) as before unmodified BoNT/A treatment, a subject treated with a modified BoNT/A according to the invention may exhibit an improvement in the equivalent one or more symptoms of at least 5%, 10%, 25%, or 50% when compared to the severity of the one or more symptoms before treatment with the modified BoNT/A. The unmodified BoNT/A is preferably SEQ ID NO: 2 present in a di-chain form.

The term “treating typical hemifacial spasm of a subject for a longer duration than that treated by an unmodified BoNT/A” may mean that one or more symptoms of said disorder of the subject are reduced for a longer time period (e.g. 6-9 months) following administration of a modified BoNT/A of the invention, when compared to administration of an unmodified BoNT/A. Said reduction may be determined by comparison to an equivalent control subject exhibiting equivalent symptoms that has been treated with an unmodified BoNT/A. At a time period where the severity of one or more symptoms of the control subject are substantially the same (e.g. the same) as before unmodified BoNT/A treatment, a subject treated with a modified BoNT/A according to the invention may exhibit an improvement in the equivalent one or more symptoms of at least 5%, 10%, 25%, or 50% when compared to the severity of the one or more symptoms before treatment with the modified BoNT/A.

The term “treating atypical hemifacial spasm of a subject for a longer duration than that treated by an unmodified BoNT/A” may mean that one or more symptoms of said disorder of the subject are reduced for a longer time period (e.g. 6-9 months) following administration of a modified BoNT/A of the invention, when compared to administration of an unmodified BoNT/A. Said reduction may be determined by comparison to an equivalent control subject exhibiting equivalent symptoms that has been treated with an unmodified BoNT/A. At a time period where the severity of one or more symptoms of the control subject are substantially the same (e.g. the same) as before unmodified BoNT/A treatment, a subject treated with a modified BoNT/A according to the invention may exhibit an improvement in the equivalent one or more symptoms of at least 5%, 10%, 25%, or 50% when compared to the severity of the one or more symptoms before treatment with the modified BoNT/A.

The unit dose may be at least 240.4 pg, 500 pg, 1,000 pg, 2,000 pg, 3,000 pg or 4,000 pg, for example at least 1,000 pg of modified BoNT/A.

The unit dose may be at least 240.4 pg, 500 pg, 1,000 pg, 2,000 pg, 3,000 pg, 4,000 pg, 5,000 pg, 6000 pg, or 7,000 pg, preferably at least 4,000 pg of modified BoNT/A.

In one embodiment, the upper limit of a unit dose of the invention may be determined based on the total dose administered during the treatment and the number of muscles and/or sites thereof to which the modified BoNT/A is administered. For example, where the total dose administered during a treatment for blepharospasm is up to 75,000 pg of modified BoNT/A and administration comprises (i) a (single) unit dose to the lateral upper orbicularis oculi muscle proximal to a first eye of the subject, (ii) a (single) unit dose to the medial upper orbicularis oculi muscle proximal to the first eye, (iii) a (single) unit dose to the lateral lower orbicularis oculi muscle proximal to the first eye only, (iv) two unit doses to the frontalis proximal to the first eye only, (v) two unit doses to a corrugator muscle proximal to the first eye only and (vi) a (single) unit dose to the procerus (e.g. eight unit doses at a plurality of sites), then the upper limit of the unit dose may be 9,375 pg. If additionally administration comprises (i) a (single) unit dose to the lateral upper orbicularis oculi muscle proximal to a second eye of the subject, (ii) a (single) unit dose to the medial upper orbicularis oculi muscle proximal to a second eye, (iii) a (single) unit dose to the lateral lower orbicularis oculi muscle proximal to a second eye, (iv) two unit doses to the frontalis proximal to a second eye, and (v) two unit doses to a corrugator muscle proximal to a second eye (e.g. an additional seven unit doses such that the total number of unit doses is 15), the upper limit may be 5,000 pg.

The unit dose may be 240 pg to 8,000 pg of modified BoNT/A, wherein the modified BoNT/A comprises a BoNT/A light-chain and translocation domain, and a BoNT/B receptor binding domain (H_{C} domain). An upper limit of the unit dose range may be 7,500, 7,000, 6,500, 6,000, 5,500, 5,000, 4,800, 4,500, 4,000, 3,500, 3,000, 2,500, 2,400, 2,000, 1,500, or 1,250 pg of modified BoNT/A. An upper limit of the unit dose range may be 5,500, 5,000, or 4,800 of modified. A preferred upper limit of the unit dose range may be 5,000 of modified BoNT/A. A lower limit of the unit dose range may be 300, 400, 500, 600, 700, 800, 900, 1,000, 1,500, 2,000, 2,500, 3,000, 3,500, 4,000, 4,500, or 5,000 pg of modified BoNT/A, preferably the lower limit is 1,000 pg. The unit dose may be 1,000 pg to 6,000 pg, 1,000 pg to 5,500 pg, 1,000 pg to 5,000 pg, or 1,000 pg to 4,500 pg of the modified BoNT/A. The unit dose may be 1,000 pg to 4,800 pg, 1,000 pg to 4,000 pg, 1,000 pg to 2,400 pg, or 1,000 pg to 2,000 pg.

The unit dose may be 1,500 to 5,000 pg of modified BoNT/A, preferably 2,000 to 4,500 pg of modified BoNT/A. Examples of suitable unit doses include about 2,500 pg (e.g. 2,000 pg±10%) of modified BoNT/A; and about 4,000 pg (e.g. 4,000 pg±10%) of modified BoNT/A. Such unit doses are particularly suitable in the treatment of blepharospasm (whether bi- or unilateral).

A total dose administered when carrying out the treatment regimen of the present invention may be greater than 24,000 pg (e.g. preferably greater than 24,100 pg; more preferably greater than 35,000 pg) of the modified BoNT/A. In other words, the total amount of the modified BoNT/A administered at a given treatment session may be greater than 24,000 pg. The total dose may be up to 82,500, 80,000, 75,000, 70,000, 65,000, 60,000, 55,000, 50,000, 45,000, 40,000, 35,000, 30,000 or 25,000 pg. The total dose may be at least 24,500, 25,000, 30,000, 35,000, 40,000, 45,000, 50,000, 55,000, 60,000, 65,000 or 70,000 pg. Preferably, the total dose may be at least 30,000 pg of modified BoNT/A. The total dose may be greater than 24,000 pg to 70,000, preferably 30,000 pg to 60,000 pg.

The total dose may be greater than 35,000 pg to 70,000 pg.

The total dose may be between 25,000 pg and 50,000 pg of modified BoNT/A (e.g. 25,000 pg to 50,000 pg). Examples of suitable ranges for the total dose include 25,000 pg to 35,000 pg of modified BoNT/A; and 45,000 pg to 50,000 pg of modified BoNT/A. Examples of suitable total doses include about 30,000 pg (e.g. 30,000 pg±10%) and about 48,000 pg (e.g. 48,000 pg±10%) of modified BoNT/A. Such unit doses are particularly suitable in the treatment of blepharospasm (whether bi- or unilateral).

Accordingly, the unit dose may be at least 240 pg of the modified BoNT/A and the total dose administered when carrying out the treatment regimen of the present invention may be greater than 24,000 pg and up to 82,500 pg of the modified BoNT/A. The unit dose may be at least 240 pg of the modified BoNT/A and the total dose administered when carrying out the treatment regimen of the present invention may be greater than 35000 pg and up to 82,500 pg of the modified BoNT/A.

In a preferable embodiment, the unit dose may be at least 240 pg of the modified BoNT/A and the total dose administered when carrying out the treatment regimen of the present invention may be greater than 24,000 pg and up to 75,000 pg of the modified BoNT/A. The unit dose may be at least 240 pg of the modified BoNT/A and the total dose administered when carrying out the treatment regimen of the present invention may be greater than 35000 pg and up to 75,000 pg of the modified BoNT/A.

In another preferable embodiment, the unit dose may be up to 5,000 pg of the modified BoNT/A and the total dose administered when carrying out the treatment regimen of the present invention may be greater than 24,000 pg and up to 75,000 pg of the modified BoNT/A. The unit dose may be up to 5,000 pg of the modified BoNT/A and the total dose administered when carrying out the treatment regimen of the present invention may be greater than 35,000 pg and up to 75,000 pg of the modified BoNT/A.

In another preferable embodiment, the unit dose may be 2,000 ng to 4,500 pg of modified BoNT/A, and total dose administered when carrying out the treatment regimen of the present invention may be 25,000 pg to 50,000 pg of the modified BoNT/A. Such regimen may be particularly suitable in the treatment of blepharospasm (whether bi- or unilateral). For example, suitable dosage regimens include:
unit dose of about 2,500 pg (e.g. 2,000 pg±10%) of modified BoNT/A and a total dose of about 30,000 pg (e.g. 30,000 pg±10%) of modified BoNT/A
unit dose about 4,000 pg (e.g. 4,000 pg±10%) of modified BoNT/A and a total dose of about 48,000 pg (e.g. 48,000 pg±10%) of modified BoNT/A.

In the case of a unilateral condition (e.g. affecting one side of the face, such as one of the eyes for unilateral blepharospasm), said total doses may refer the total dose administered to the side of the face that is affected by the condition. In the case of a bilateral condition (e.g. affecting both sides of the face, such as both eyes for unilateral blepharospasm), said total doses may refer to the total dose that is administered across both sides of the face. It preferred that 50% of the total dose be administered to each side of the face (e.g. each eye in the case of bilateral blepharospasm).

One aspect provides a modified botulinum neurotoxin A (BoNT/A) for use in a method of treating blepharospasm in a subject, wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the lateral lower preseptal orbicularis oculi muscle) proximal to the first eye of the subject,
   wherein the unit dose of the modified BoNT/A is at least 10 Units (U) of modified BoNT/A, wherein 1 Unit is an amount of the modified BoNT/A that corresponds to the calculated median lethal dose (LD₅₀) in mice,
   wherein the total dose administered during the treatment is greater than 998 U and up to 3,432 U (e.g. up to 3,120 U) of the modified BoNT/A, and
   wherein the modified BoNT/A comprises a BoNT/A light-chain and translocation domain, and a BoNT/B receptor binding domain (H_{C} domain).

In a related aspect, the invention provides a modified botulinum neurotoxin A (BoNT/A) for use in a method of treating blepharospasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the lateral lower preseptal orbicularis oculi muscle) proximal to the first eye of the subject,
   wherein the unit dose of the modified BoNT/A is at least 10 Units (U) of modified BoNT/A, wherein 1 Unit is an amount of the modified BoNT/A that corresponds to the calculated median lethal dose (LD50) in mice,
   wherein the total dose administered during the treatment is greater than 998 U and up to 3,432 U (e.g. up to 3,120 U) of the modified BoNT/A, and
   wherein the modified BoNT/A comprises a BoNT/A light-chain and translocation domain, and a BoNT/B receptor binding domain (HC domain).

In a related aspect, the invention provides a method of treating blepharospasm in a subject, wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the lateral lower preseptal orbicularis oculi muscle) proximal to the first eye of the subject,
   wherein the unit dose of the modified BoNT/A is at least 10 Units (U) of modified BoNT/A, wherein 1 Unit is an amount of the modified BoNT/A that corresponds to the calculated median lethal dose (LD50) in mice,
   wherein the total dose administered during the treatment is greater than 998 U and up to 3,432 U (e.g. up to 3,120 U) of the modified BoNT/A, and
   wherein the modified BoNT/A comprises a BoNT/A light-chain and translocation domain, and a BoNT/B receptor binding domain (HC domain).

In a related aspect, the invention provides a method of treating blepharospasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the lateral lower preseptal orbicularis oculi muscle) proximal to the first eye of the subject,
   wherein the unit dose of the modified BoNT/A is at least 10 Units (U) of modified BoNT/A, wherein 1 Unit is an amount of the modified BoNT/A that corresponds to the calculated median lethal dose (LD50) in mice,
   wherein the total dose administered during the treatment is greater than 998 U and up to 3,432 U (e.g. up to 3,120 U) of the modified BoNT/A, and
   wherein the modified BoNT/A comprises a BoNT/A light-chain and translocation domain, and a BoNT/B receptor binding domain (HC domain).

In one aspect, the invention provides the use of a modified botulinum neurotoxin A (BoNT/A) in the manufacture of medicament for treating blepharospasm in a subject, comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the lateral lower preseptal orbicularis oculi muscle) proximal to the first eye of the subject,
   wherein the unit dose of the modified BoNT/A is at least 10 Units (U) of modified BoNT/A, wherein 1 Unit is an amount of the modified BoNT/A that corresponds to the calculated median lethal dose (LD50) in mice,
   wherein the total dose administered during the treatment is greater than 998 U and up to 3,432 U (e.g. up to 3,120 U) of the modified BoNT/A, and
   wherein the modified BoNT/A comprises a BoNT/A light-chain and translocation domain, and a BoNT/B receptor binding domain (HC domain).

In one aspect, the invention provides the use of a modified botulinum neurotoxin A (BoNT/A) in the manufacture of medicament for treating blepharospasm in a subject for a longer duration than that treated by an unmodified BoNT/A (e.g. SEQ ID NO: 2 [such as a di-chain form of SEQ ID NO: 2]), comprising:
a) administering a unit dose of the modified BoNT/A to the lateral upper orbicularis oculi muscle (preferably the lateral upper preseptal orbicularis oculi muscle) proximal to a first eye of the subject;
b) administering a unit dose of the modified BoNT/A to the medial upper orbicularis oculi muscle (preferably the medial upper preseptal orbicularis oculi muscle) proximal to the first eye of the subject; and
c) administering a unit dose of the modified BoNT/A to the lateral lower orbicularis oculi muscle (preferably the medial upper preseptal orbicularis oculi muscle) proximal to
   the first eye of the subject,
   wherein the unit dose of the modified BoNT/A is at least 10 Units (U) of modified BoNT/A, wherein 1 Unit is an amount of the modified BoNT/A that corresponds to the calculated median lethal dose (LD50) in mice,
   wherein the total dose administered during the treatment is greater than 998 U and up to 3,432 U (e.g. up to 3,120 U) of the modified BoNT/A, and
   wherein the modified BoNT/A comprises a BoNT/A light-chain and translocation domain, and a BoNT/B receptor binding domain (HC domain).

Another aspect provides a modified botulinum neurotoxin A (BoNT/A) for use in a method of treating typical hemifacial spasm, wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle (preferably the lateral lower preseptal orbicularis oculi muscle) proximal to an eye affected by hemifacial spasm; and
d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
wherein the unit dose of the modified BoNT/A is at least 10 Units (U) of modified BoNT/A, wherein 1 Unit is an amount of the modified BoNT/A that corresponds to the calculated median lethal dose (LD50) in mice,
wherein the total dose administered during the treatment is greater than 998 U and up to 3,432 U (e.g. up to 3,120 U) of the modified BoNT/A, and
wherein the modified BoNT/A comprises a BoNT/A light-chain and translocation domain, and a BoNT/B receptor binding domain (HC domain).

In a related aspect, the invention provides a modified botulinum neurotoxin A (BoNT/A) for use in a method of treating typical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle (preferably the lateral lower preseptal orbicularis oculi muscle) proximal to an eye affected by hemifacial spasm; and
d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
wherein the unit dose of the modified BoNT/A is at least 10 Units (U) of modified BoNT/A, wherein 1 Unit is an amount of the modified BoNT/A that corresponds to the calculated median lethal dose (LD50) in mice,
wherein the total dose administered during the treatment is greater than 998 U and up to 3,432 U (e.g. up to 3,120 U) of the modified BoNT/A, and
wherein the modified BoNT/A comprises a BoNT/A light-chain and translocation domain, and a BoNT/B receptor binding domain (HC domain).

In a related aspect, the invention provides a method of treating typical hemifacial spasm in a subject, wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle (preferably the lateral lower preseptal orbicularis oculi muscle) proximal to an eye affected by hemifacial spasm; and
d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
wherein the unit dose of the modified BoNT/A is at least 10 Units (U) of modified BoNT/A, wherein 1 Unit is an amount of the modified BoNT/A that corresponds to the calculated median lethal dose (LD50) in mice,
wherein the total dose administered during the treatment is greater than 998 U and up to 3,432 U (e.g. up to 3,120 U) of the modified BoNT/A, and
wherein the modified BoNT/A comprises a BoNT/A light-chain and translocation domain, and a BoNT/B receptor binding domain (HC domain).

In a related aspect, the invention provides a method of treating typical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle (preferably the lateral lower preseptal orbicularis oculi muscle) proximal to an eye affected by hemifacial spasm; and
d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
wherein the unit dose of the modified BoNT/A is at least 10 Units (U) of modified BoNT/A, wherein 1 Unit is an amount of the modified BoNT/A that corresponds to the calculated median lethal dose (LD50) in mice,
wherein the total dose administered during the treatment is greater than 998 U and up to 3,432 U (e.g. up to 3,120 U) of the modified BoNT/A, and
wherein the modified BoNT/A comprises a BoNT/A light-chain and translocation domain, and a BoNT/B receptor binding domain (HC domain).

In one aspect, the invention provides the use of a modified botulinum neurotoxin A (BoNT/A) in the manufacture of medicament for treating typical hemifacial spasm in a subject, wherein the modified BoNT/A is administered by intramuscular injection at a plurality of sites of the face of the subject, comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle (preferably the lateral lower preseptal orbicularis oculi muscle) proximal to an eye affected by hemifacial spasm; and
d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
      wherein the unit dose of the modified BoNT/A is at least 10 Units (U) of modified BoNT/A, wherein 1 Unit is an amount of the modified BoNT/A that corresponds to the calculated median lethal dose (LD50) in mice,
      wherein the total dose administered during the treatment is greater than 998 U and up to 3,432 U (e.g. up to 3,120 U) of the modified BoNT/A, and
      wherein the modified BoNT/A comprises a BoNT/A light-chain and translocation domain, and a BoNT/B receptor binding domain (HC domain).

In one aspect, the invention provides the use of a modified botulinum neurotoxin A (BoNT/A) in the manufacture of medicament for treating typical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, comprising:
a) administering a unit dose of the modified BoNT/A to the lateral upper orbicularis oculi muscle (preferably the lateral upper preseptal orbicularis oculi muscle) proximal to an eye affected by hemifacial spasm;
b) administering a unit dose of the modified BoNT/A to the medial upper orbicularis oculi muscle (preferably the medial upper preseptal orbicularis oculi muscle) proximal to an eye affected by hemifacial spasm;
c) administering a unit dose of the modified BoNT/A to the lateral lower orbicularis oculi muscle (preferably the lateral lower preseptal orbicularis oculi muscle) proximal to an eye affected by hemifacial spasm; and
d) administering one or more unit dose of the modified BoNT/A to one or more further muscles affected by said hemifacial spasm in accordance with the following dosage regimen:
   (i) one unit dose to an orbicularis oris upper muscle;
   (ii) one unit dose to an orbicularis oris lower muscle;
   (iii) one unit dose to a zygomaticus major muscle;
   (iv) one unit dose to a zygomaticus minor muscle;
   (v) up to five unit doses (preferably one unit dose) to a frontalis muscle;
   (vi) one unit dose to a mentalis muscle;
   (vii) one unit dose to a platysma muscle;
   (viii) up to two unit doses (preferably one unit dose) to a corrugator muscle;
   (ix) one unit dose to a buccinator muscle;
   (x) up to two unit doses (preferably one unit dose) to a masseter muscle;
   (xi) one unit dose to a procerus muscle;
   (xii) one unit dose to a nasalis muscle; and/or
   (xiii) one unit dose to a levator palpebrae superiori muscle;
wherein the unit dose of the modified BoNT/A is at least 10 Units (U) of modified BoNT/A, wherein 1 Unit is an amount of the modified BoNT/A that corresponds to the calculated median lethal dose (LD50) in mice,
wherein the total dose administered during the treatment is greater than 998 U and up to 3,432 U (e.g. up to 3,120 U) of the modified BoNT/A, and
wherein the modified BoNT/A comprises a BoNT/A light-chain and translocation domain, and a BoNT/B receptor binding domain (HC domain).

Another aspect provides a modified botulinum neurotoxin A (BoNT/A) for use in a method of treating atypical hemifacial spasm, wherein the modified BoNT/A is administered by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicul; and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 10 Units (U) of modified BoNT/A, wherein 1 Unit is an amount of the modified BoNT/A that corresponds to the calculated median lethal dose (LD50) in mice,
wherein the total dose administered during the treatment is greater than 998 U and up to 3,432 U (e.g. up to 3,120 U) of the modified BoNT/A, and
wherein the modified BoNT/A comprises a BoNT/A light-chain and translocation domain, and a BoNT/B receptor binding domain (HC domain).

In a related aspect, the invention provides a modified botulinum neurotoxin A (BoNT/A) for use in a method of treating atypical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicul; and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 10 Units (U) of modified BoNT/A, wherein 1 Unit is an amount of the modified BoNT/A that corresponds to the calculated median lethal dose (LD50) in mice,
wherein the total dose administered during the treatment is greater than 998 U and up to 3,432 U (e.g. up to 3,120 U) of the modified BoNT/A, and
wherein the modified BoNT/A comprises a BoNT/A light-chain and translocation domain, and a BoNT/B receptor binding domain (HC domain).

In a related aspect, the invention provides a method of treating atypical hemifacial spasm in a subject, wherein the modified BoNT/A is administered by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicul; and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 10 Units (U) of modified BoNT/A, wherein 1 Unit is an amount of the modified BoNT/A that corresponds to the calculated median lethal dose (LD50) in mice,
wherein the total dose administered during the treatment is greater than 998 U and up to 3,432 U (e.g. up to 3,120 U) of the modified BoNT/A, and
wherein the modified BoNT/A comprises a BoNT/A light-chain and translocation domain, and a BoNT/B receptor binding domain (HC domain).

In a related aspect, the invention provides a method of treating atypical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicul; and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 10 Units (U) of modified BoNT/A, wherein 1 Unit is an amount of the modified BoNT/A that corresponds to the calculated median lethal dose (LD50) in mice, wherein the total dose administered during the treatment is greater than 998 U and up to 3,432 U (e.g. up to 3,120 U) of the modified BoNT/A, and
wherein the modified BoNT/A comprises a BoNT/A light-chain and translocation domain, and a BoNT/B receptor binding domain (HC domain).

In one aspect, the invention provides the use of a modified botulinum neurotoxin A (BoNT/A) in the manufacture of medicament for treating atypical hemifacial spasm in a subject, wherein the modified BoNT/A is administered by intramuscular injection at a plurality of sites of the face of the subject, comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicul; and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 10 Units (U) of modified BoNT/A, wherein 1 Unit is an amount of the modified BoNT/A that corresponds to the calculated median lethal dose (LD50) in mice,
wherein the total dose administered during the treatment is greater than 998 U and up to 3,432 U (e.g. up to 3,120 U) of the modified BoNT/A, and
wherein the modified BoNT/A comprises a BoNT/A light-chain and translocation domain, and a BoNT/B receptor binding domain (HC domain).

In one aspect, the invention provides the use of a modified botulinum neurotoxin A (BoNT/A) in the manufacture of medicament for treating atypical hemifacial spasm in a subject for a longer duration than that treated by an unmodified BoNT/A (e.g. SEQ ID NO: 2 [such as a di-chain form of SEQ ID NO: 2]), wherein the modified BoNT/A is administered by intramuscular injection at a plurality of sites of the face of the subject, comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicul; and
b) optionally administering one or more unit dose of the modified BoNT/A to one or more further muscles affected by said hemifacial spasm in accordance with the following dosage regimen:
   (i) one unit dose to a zygomaticus major muscle;
   (ii) one unit dose to a zygomaticus major muscle;
   (iii) up to five unit doses (preferably one unit dose) to a frontalis muscle;
   (iv) one unit dose to a mentalis muscle;
   (v) one unit dose to a platysma muscle;
   (vi) up to two unit doses (preferably one unit dose) to a corrugator muscle;
   (vii) one unit dose to a buccinator muscle;
   (viii) up to two unit doses (preferably one unit dose) to a masseter muscle;
   (ix) one unit dose to a procerus muscle;
   (x) one unit dose to a nasalis muscle;
   (xi) one unit dose to a lateral upper orbicularis oculi muscle;
   (xii) one unit dose to a medial upper orbicularis oculi muscle;
   (xiii) one unit dose to a lateral lower orbicularis oculi muscle; and/or
   (xiv) one unit dose to a levator palpebrae superiori muscle;
wherein the unit dose of the modified BoNT/A is at least 10 Units (U) of modified BoNT/A, wherein 1 Unit is an amount of the modified BoNT/A that corresponds to the calculated median lethal dose (LD50) in mice,
wherein the total dose administered during the treatment is greater than 998 U and up to 3,432 U (e.g. up to 3,120 U) of the modified BoNT/A, and
wherein the modified BoNT/A comprises a BoNT/A light-chain and translocation domain, and a BoNT/B receptor binding domain (HC domain).

The unit dose may be at least 21 U, 42 U, 83 U, 125 U, or 166 U, preferably at least 42 U, wherein the modified BoNT/A comprises a BoNT/A light-chain and translocation domain, and a BoNT/B receptor binding domain (H_{C} domain).

The unit dose may be 10 U to 332.7 U of modified BoNT/A, wherein the modified BoNT/A comprises a BoNT/A light-chain and translocation domain, and a BoNT/B receptor binding domain (H_{C} domain). An upper limit of the unit dose range may be 312, 291, 270, 250, 229, 208, 199.6, 187, 166.3, 146, 125, 104, 99.8, 89.17, 62, or 52 U of modified BoNT/A. A lower limit of the unit dose range may be 12, 17, 21, 25, 29, 33, 37, 42, 62, 83, 104, 125, 146, 166, 187, or 208 U of modified BoNT/A, preferably the lower limit is 42 U. The unit dose may be 42 U to 199.6 U, 42 U to 166.3 U, 42 U to 99.8 U, or 42 U to 83.17 U.

The unit dose may be 62.4 to 208 U of modified BoNT/A, preferably 83.2 to 187.2 U of modified BoNT/A. Examples of suitable unit doses include about 104 U (e.g. 104 U±10%) of modified BoNT/A; and about 166.4 U (e.g. 166.4 U±10%) of modified BoNT/A. Such unit doses are particularly suitable in the treatment of blepharospasm (whether bi- or unilateral).

A total dose administered when carrying out the treatment regimen of the present invention may be greater than 998U of the modified BoNT/A. In other words, the total amount of the modified BoNT/A administered at a given treatment session may be greater than 998U. The total dose may be up to 3430U, 3326U, 3119U, 2911U, 2703U, 2495U, 2287U, 2079U, 1871U, 1663U, 1455U, 1247U or 1040U. The total dose may be at least 1019U, 1040U, 1247U, 1455U, 1663U, 1871U, 2079U, 2287U, 2495U, 2703U or 2911U. Preferably, the total dose may be at least 125U of modified BoNT/A. The total dose may be greater than 998U to 2911U, preferably 1247U to 2495U.

The total dose may be between 1,040 U and 2080 U of modified BoNT/A. Examples of suitable ranges for the total dose include 1,040 U to 1456 U of modified BoNT/A; and 1871.9 U to 2079.9 U of modified BoNT/A. Examples of suitable total doses include about 1247.9 U (e.g. 1247.9 U±10%) of modified BoNT/A; and about 1996.7 U (e.g. 1996.7 U±10%) of modified BoNT/A. Such unit doses are particularly suitable in the treatment of blepharospasm (whether bi- or unilateral).

Accordingly, the unit dose may be at least 10U of the modified BoNT/A and the total dose administered when carrying out the treatment regimen of the present invention may be greater than 998U and up to 3430U of the modified BoNT/A. In a preferable embodiment, the unit dose may be at least 10U of the modified BoNT/A and the total dose administered when carrying out the treatment regimen of the present invention may be greater than 998U and up to 3119U of the modified BoNT/A. In another preferable embodiment, the unit dose may be up to 208U of the modified BoNT/A and the total dose administered when carrying out the treatment regimen of the present invention may be greater than 998U and up to 3119U of the modified BoNT/A

In another preferable embodiment, the unit dose may be 83.2 U to 187.2 U of modified BoNT/A, and total dose administered when carrying out the treatment regimen of the present invention may be 1039.9 U to 2079.9 U of the modified BoNT/A. Such regimen may be particularly suitable in the treatment of blepharospasm (whether bi- or unilateral). For example, suitable dosage regimens include:
unit dose of about 104 U (e.g. 104 U±10%) of modified BoNT/A and a total dose of about 1247.9 U (e.g. 1247.9 U±10%) of modified BoNT/A
unit dose about 166.4 U (e.g. 166.4 U±10%) of modified BoNT/A and a total dose of about 1996.7 U (e.g. 1996.7 U±10%) of modified BoNT/A.

In the case of a unilateral condition (e.g. affecting one side of the face, such as one of the eyes for unilateral blepharospasm), said total doses may refer the total dose administered to the side of the face that is affected by the condition. In the case of a bilateral condition (e.g. affecting both sides of the face, such as both eyes for unilateral blepharospasm), said total doses may refer to the total dose that is administered across both sides of the face. It preferred that 50% of the total dose be administered to each side of the face (e.g. each eye in the case of bilateral blepharospasm).

Suitable disorders affecting an eyelid muscle (e.g. affecting two or more eyelid muscles) include blepharospasm and facial spasm (e.g. hemifacial spasm). Preferably, a disorder affecting an eyelid muscle of a subject is blepharospasm. Thus, the present invention may be directed to the treatment of blepharospasm and/or facial spasm (e.g. hemifacial spasm), preferably directed to the treatment of blepharospasm.

The present invention is predicated on the surprising outcome of dose escalation studies that demonstrate that high dose treatment of a clostridial neurotoxin (e.g. greater than 24,000 pg and up to 82,500 pg) may be employed to treat these disorders, without exceeding acceptable toxicity levels (see the examples).

A disorder affecting an eyelid muscle of a subject may be an eyelid muscle disorder. The cause of the disorder may be a nerve-related disorder (e.g. a VIIth nerve disorder).

A modified BoNT/A may be administered to any muscle that is affected by the disorder (e.g. an affected eyelid muscle). The affected muscle may contribute to (e.g. cause) one or more symptoms of the disorder (e.g. blepharospasm and/or facial spasm, such as hemifacial spasm).

In one embodiment, (preferably embodiments directed to treating blepharospasm) a single unit dose only of modified BoNT/A is administered to at least each of: the lateral upper orbicularis oculi muscle (e.g. the lateral pretarsal orbicularis oculi of the upper lid) proximal to a first eye of the subject; the medial upper orbicularis oculi muscle (e.g. the medial pretarsal orbicularis oculi of the upper lid) proximal to the first eye of the subject; and the lateral lower orbicularis oculi muscle (e.g. the lateral pretarsal orbicularis oculi of the lower lid) proximal to the first eye of the subject. Preferably, a single unit is administered per injection site, which, in this embodiment may correspond to administration at three injection sites. Thus, three unit doses may be administered according to the above, however further muscles and/or sites thereof may be treated in accordance with the invention, meaning that the total number of unit doses administered may be greater than three.

In one embodiment, (preferably embodiments directed to treating blepharospasm) where the disorder affects eyelid muscles proximal to both eyes of the subject (e.g. bilateral blepharospasm), a single unit dose only of modified BoNT/A may be administered to at least each of: the lateral upper orbicularis oculi muscle proximal to a first eye of the subject; the medial upper orbicularis oculi muscle proximal to the first eye of the subject; the lateral lower orbicularis oculi muscle proximal to the first eye of the subject; the lateral upper orbicularis oculi muscle proximal to a second eye of the subject; the medial upper orbicularis oculi muscle proximal to the second eye of the subject; and the lateral lower orbicularis oculi muscle proximal to the second eye of the subject. Preferably, a single unit is administered per injection site, which, in this embodiment may correspond to administration at six injection sites. Thus, six unit doses may be administered according to the above, however further muscles and/or sites thereof may be treated in accordance with the invention, meaning that the total number of unit doses administered may be greater than six.

The terms “first eye” and “second eye” may refer to either the left eye or the right eye. The terms simply serve to distinguish the two eyes from one another. In other words, if the first eye is the left eye, then the second eye will be the right eye, and vice versa. Reference to a “first eye” is not intended to imply that muscles and/or sites thereof proximal to a “second eye” need always be treated. For example, a “first eye” may be referred to in the context of a unilateral disorder, e.g. unilateral blepharospasm, where muscles and/or sites thereof proximal to a second eye are not affected and, thus, are not treated.

The term “proximal” means that a muscle and/or site thereof referred to is nearest to the eye mentioned. For example, if the first eye is the left eye of a subject, then a muscle and/or site thereof that is proximal to said first eye is a muscle and/or site thereof that is closer to the left eye than to the right eye of the subject.

A modified BoNT/A may be administered to one or more further muscles and/or sites thereof. When administering to further muscles and/or sites thereof, the upper limit of a unit dose is preferably set to ensure that the total amount of modified BoNT/A administered does not exceed a total dose to be administered during treatment as defined according to the invention.

Additional muscles and/or sites thereof treated may be one or more (e.g. at least two, three, four, five, six, seven, eight, nine, ten, eleven, or twelve, or all muscles and/or sites) selected from: the medial lower orbicularis oculi muscle, the orbicularis oris (e.g. the orbicularis oris upper and/or the orbicularis oris lower); the zygomaticus (e.g. zygomaticus major); the nasalis; the mentalis; the platysma; the frontalis; the corrugator; the buccinator; the masseter; the procerus; and the lateral canthus. Additional muscles and/or sites thereof treated may be one or more (e.g. at least two, three, four, five, six, seven, eight, nine, ten, eleven, or twelve, or all muscles and/or sites) selected from: the medial lower orbicularis oculi muscle, the orbicularis oris upper muscle, the orbicularis oris lower muscle, the zygomaticus major muscle, the zygomaticus minor muscle, the frontalis muscle, the mentalis muscle, the platysma muscle, the corrugator muscle, the buccinator muscle, the masseter muscle, the procerus muscle, the nasalis muscle, and the levator palpebrae superiori muscle. Additional muscles and/or sites thereof treated may be one or more (e.g. at least two, three, four, five, six, seven, eight, nine, ten, eleven, or twelve, or all muscles and/or sites) selected from: the orbicularis oris upper muscle, the orbicularis oris lower muscle, the zygomaticus major muscle, the zygomaticus minor muscle, the frontalis muscle, the mentalis muscle, the platysma muscle, the corrugator muscle, the buccinator muscle, the masseter muscle, the procerus muscle, the nasalis muscle, and the levator palpebrae superiori muscle.

Where a muscle is listed for administration in accordance with a treatment of the invention, the invention may further comprise administering an additional, unlisted muscle.

A muscle and/or site thereof proximal to one or both eyes may be treated as necessary. At least a single unit dose may be administered to said muscles and/or sites thereof, for example two or more (e.g. three or more, four or more or five or more) unit doses may be administered.

A modified BoNT/A may also be administered to the medial lower orbicularis oculi muscle (e.g. the medial pretarsal orbicularis oculi of the lower lid). In one embodiment, a single unit dose only may be administered to the medial lower orbicularis oculi muscle. The medial lower orbicularis oculi muscle proximal to one or both eyes may be treated as necessary.

A modified BoNT/A may also be administered to the frontalis muscle. In one embodiment, at least a single unit dose only may be administered to the frontalis muscle, e.g. two or more, three or more, four or more or five or more unit doses may be administered. The frontalis muscle proximal to one or both eyes may be treated as necessary.

A modified BoNT/A may also be administered to the corrugator muscle. In one embodiment, at least a single unit dose only may be administered to the corrugator muscle, e.g. two or more, three or more, four or more or five or more unit doses may be administered. The corrugator muscle proximal to one or both eyes may be treated as necessary.

When treating facial spasm, one or more (e.g. at least two, three, four, five, six, seven, eight, nine, ten, or eleven, or all) additional muscles and/or sites thereof may be treated, wherein the one or more muscles and/or sites thereof are selected from: the orbicularis oris (e.g. the orbicularis oris upper and/or the orbicularis oris lower); the zygomaticus (e.g. zygomaticus major); the nasalis; the mentalis; the platysma; the frontalis; the corrugator; the buccinator; the masseter; the procerus; and the lateral canthus. When treating facial spasm, one or more (e.g. at least two, three, four, five, six, seven, eight, nine, ten, or eleven, or all) additional muscles and/or sites thereof may be treated, wherein the one or more muscles and/or sites thereof are selected from: the orbicularis oris (e.g. the orbicularis oris upper and/or the orbicularis oris lower); the zygomaticus (e.g. zygomaticus major and/or zygomaticus minor); the nasalis; the mentalis; the platysma; the frontalis; the corrugator; the buccinator; the masseter; the procerus; and the levator palpebrae superiori muscle.

Preferably, one or more (e.g. at least two, three or four, or all) additional muscles and/or sites selected from: the corrugator, the frontalis, the zygomaticus major, the buccinators, and the masseter.

Where the facial spasm is bilateral, a modified BoNT/A may be administered to any muscle and/or site thereof on both sides of the subject's face. Where the facial spasm is hemifacial spasm, a modified BoNT/A may be administered to any muscle and/or site thereof on the affected side of the subject's face. At least a single unit dose may be administered to said muscles and/or sites thereof, for example two or more (e.g. three or more, four or more or five or more) unit doses may be administered.

A frontalis muscle may be a venter frontalis muscle.

A corrugator muscle may be a corrugator supercilii muscle.

In any aspect or embodiment of the invention directed to treatment of blepharospasm, it is particularly preferred that the modified BoNT/A is administered by intramuscular injection to each of the following sites:
a) the lateral upper orbicularis oculi muscle;
b) the medial upper orbicularis oculi muscle; and
c) the lateral lower orbicularis oculi muscle.

Said (three) sites outlined in the paragraph directly above may be referred to as the “minimum” sites in the treatment of blepharospasm.

In other words, in any aspect or embodiment of the invention directed to treatment of blepharospasmit is particularly preferred that the modified BoNT/A is administered by intramuscular injection to each of the following muscles:
a) the lateral upper orbicularis oculi muscle affected by said blepharospasm;
b) the medial upper orbicularis oculi muscle affected by said blepharospasm; and
c) the lateral lower orbicularis oculi muscle affected by said blepharospasm.

In yet other words, in any aspect or embodiment of the invention directed to treatment of blepharospasm, it is particularly preferred that the modified BoNT/A is administered by intramuscular injection to each of the following muscles:
a) each lateral upper orbicularis oculi muscle affected by said blepharospasm;
b) each medial upper orbicularis oculi muscle affected by said blepharospasm; and
c) each lateral lower orbicularis oculi muscle affected by said blepharospasm.

For example, where treating blepharospasm, it is particularly preferred that the method comprises:
a) administering up to two unit doses of the modified BoNT/A to the lateral upper orbicularis oculi muscle proximal to a first eye of the subject;
b) administering a unit dose of the modified BoNT/A to the medial upper orbicularis oculi muscle proximal to the first eye of the subject; and
c) administering a unit dose of the modified BoNT/A to the lateral lower orbicularis oculi muscle proximal to the first eye of the subject.

In other words, where treating blepharospasm, it is particularly preferred that the method comprises:
a) administering up to two unit doses of the modified BoNT/A to each lateral upper orbicularis oculi muscle affected by said blepharospasm.
b) administering a unit dose of the modified BoNT/A to each medial upper orbicularis oculi muscle affected by said blepharospasm; and
c) administering a unit dose of the modified BoNT/A to each lateral lower orbicularis oculi muscle affected by said blepharospasm.

For example, where treating blepharospasm, it is particularly preferred that the method comprises:
a) administering two unit doses of the modified BoNT/A to the lateral upper orbicularis oculi muscle proximal to a first eye of the subject;
b) administering a single unit dose of the modified BoNT/A to the medial upper orbicularis oculi muscle proximal to the first eye of the subject; and
c) administering a single unit dose of the modified BoNT/A to the lateral lower orbicularis oculi muscle proximal to the first eye of the subject.

In other words, where treating blepharospasm, it is particularly preferred that the method comprises:
a) administering two unit doses of the modified BoNT/A to each lateral upper orbicularis oculi muscle affected by said blepharospasm.
b) administering a single unit dose of the modified BoNT/A to each medial upper orbicularis oculi muscle affected by said blepharospasm; and
c) administering a single unit dose of the modified BoNT/A to each lateral lower orbicularis oculi muscle affected by said blepharospasm.

Reference to an “upper” orbicularis oculi muscle refers to an orbicularis oculi muscle of an upper eyelid. Similarly, reference to a “lower” orbicularis oculi muscle refers an orbicularis oculi muscle of a lower eyelid.

The skilled person understands that the term “medial” (e.g. in the context of anatomy) means toward the midline of the body. Similarly, the skilled person understands that the term “lateral” (e.g. in the context of anatomy) means away from the midline of the body. Thus:
the “lateral” upper orbicularis oculi muscle refers to a site of the orbicularis oculi muscle, of an upper eyelid, that is positioned away from the midline of the body (see, for example, positions (1) and (2) of FIG. 8)
the “medial” upper orbicularis oculi muscle refers to a site of the orbicularis oculi muscle, of an upper eyelid, that is positioned toward the midline of the body (see, for example, positions (2) of FIG. 8)
the “lateral” lower orbicularis oculi muscle refers to a site of the orbicularis oculi, of a lower eyelid, that is positioned away from the midline of the body (see, for example, positions (4) of FIG. 8).

The term “lateral upper orbicularis oculi muscle” may be used synonymously with the term “the external part of an orbicularis oculi muscle of an upper eyelid”. The term “medial upper orbicularis oculi muscle” may be used synonymously with the term “the inner part of an orbicularis oculi muscle of an upper eyelid”. The term “lateral lower orbicularis oculi muscle” may be used synonymously with the term “the external part of an orbicularis oculi muscle of a lower eyelid”.

An orbicularis oculi muscle comprises a “pretarsal portion” and a “preseptal portion” (either of which can be injected into).

Thus, administration to a lateral upper orbicularis oculi muscle may mean administering to a lateral “pretarsal” upper orbicularis oculi muscle, or to a lateral “preseptal” upper orbicularis oculi muscle.

Administration to a medial upper orbicularis oculi muscle may mean administration to a medial upper “pretarsal” orbicularis oculi muscle, or to a medial upper “preseptal” orbicularis oculi muscle.

Administration to a lateral lower orbicularis oculi muscle may mean administration to a lateral lower “pretarsal” orbicularis oculi muscle, or to a lateral lower “preseptal” orbicularis oculi muscle.

When administering to a lateral upper orbicularis oculi muscle, it is preferred that said unit dose is administered to the preseptal portion (in other words, to a lateral “preseptal” upper orbicularis oculi muscle). When administering to a medial upper orbicularis oculi muscle, it is preferred that said unit dose is administered to the preseptal portion (in other words, to a medial upper “preseptal” orbicularis oculi muscle). When administering to a lateral lower orbicularis oculi muscle, it is preferred that said unit dose is administered to the preseptal portion (in other words, to a lateral lower “preseptal” orbicularis oculi muscle).

In any aspect or embodiment of the invention directed to treatment of blepharospasm, the invention may further comprise administering one or more unit dose of the modified BoNT/A to one or more further muscles affected by said blepharospasm in accordance with the following dosage regimen:
(i) one unit dose to an orbicularis oris upper muscle;
(ii) one unit dose to an orbicularis oris lower muscle;
(iii) one unit dose to a zygomaticus major muscle;
(iv) one unit dose to a zygomaticus minor muscle;
(v) up to five unit doses (preferably one unit dose) to a frontalis muscle;
(vi) one unit dose to a mentalis muscle;
(vii) one unit dose to a platysma muscle;
(viii) up to two unit doses (preferably one unit dose) to a corrugator muscle;
(ix) one unit dose to a buccinator muscle;
(x) up to two unit doses (preferably one unit dose) to a masseter muscle;
(xi) one unit dose to a procerus muscle;
(xii) one unit dose to a nasalis muscle; and/or
(xiii) one unit dose to a levator palpebrae superiori muscle.

In any aspect or embodiment of the invention directed to treatment of blepharospasm, the invention may further comprise administering one or more unit dose of the modified BoNT/A to one or more further muscles affected by said blepharospasm in accordance with the following dosage regimen:
(i) up to two unit doses (preferably one unit dose) to a corrugator muscle;
(ii) up to five unit doses (preferably one unit dose) to a frontalis muscle; and/or
(iii) one unit dose to a procerus muscle.

In other words, in any aspect or embodiment of the invention directed to treatment of blepharospasm, the invention may further comprise administering one or more unit dose of the modified BoNT/A to one or more further muscles affected by said blepharospasm in accordance with the following dosage regimen:
(i) up to two unit doses (preferably one unit dose) per corrugator muscle (e.g. that is affected by said blepharospasm);
(ii) up to five unit doses (preferably one unit dose) to a frontalis muscle; and/or
(iii) one unit dose to a procerus muscle.

In any aspect or embodiment of the invention directed to treatment of blepharospasm, the invention may further comprise administering one or more unit dose of the modified BoNT/A to one or more further muscles affected by said blepharospasm in accordance with the following dosage regimen:
(i) up to two unit doses (preferably one unit dose) to a corrugator muscle; or one unit dose to a procerus muscle; and/or
(ii) up to five unit doses (preferably one unit dose) to a frontalis muscle.

In other words, in any aspect or embodiment of the invention directed to treatment of blepharospasm, the invention may further comprise administering one or more unit dose of the modified BoNT/A to one or more further muscles affected by said blepharospasm in accordance with the following dosage regimen:
(i) up to two unit doses (preferably one unit dose) per corrugator muscle (e.g. that is affected by said blepharospasm); or one unit dose to a procerus muscle; and/or
(ii) up to five unit doses (preferably one unit dose) to a frontalis muscle.

In any aspect or embodiment of the invention directed to treatment of blepharospasm, the invention may further comprise administering one or more unit dose of the modified BoNT/A to one or more further muscles affected by said blepharospasm in accordance with the following dosage regimen:
(i) up to two unit doses (preferably one unit dose) to a corrugator muscle, and/or one unit dose to a procerus muscle; and optionally
(ii) up to five unit doses (preferably one unit dose) to a frontalis muscle.

In other words, in any aspect or embodiment of the invention directed to treatment of blepharospasm, the invention may further comprise administering one or more unit dose of the modified BoNT/A to one or more further muscles affected by said blepharospasm in accordance with the following dosage regimen:
(i) up to two unit doses (preferably one unit dose) per corrugator muscle (e.g. that is affected by said blepharospasm), and/or one unit dose to a procerus muscle; and optionally
(ii) up to five unit doses (preferably one unit dose) to a frontalis muscle.

In any aspect or embodiment of the invention directed to treatment of blepharospasm, the invention may further comprise administering the modified BoNT/A to further muscles affected by said blepharospasm in accordance with the following dosage regimen:
(i) up to two unit doses (preferably one unit dose) to a corrugator muscle;
(ii) up to five unit doses (preferably one unit dose) to a frontalis muscle; and
(iii) one unit dose to a procerus muscle.

In other words, in any aspect or embodiment of the invention directed to treatment of blepharospasm, the invention may further comprise administering the modified BoNT/A to further muscles affected by said blepharospasm in accordance with the following dosage regimen:
(i) up to two unit doses (preferably one unit dose) per corrugator muscle (e.g. that is affected by said blepharospasm);
(ii) up to five unit doses (preferably one unit dose) to a frontalis muscle; and
(iii) one unit dose to a procerus muscle.

In any aspect or embodiment of the invention directed to treatment of blepharospasm, the invention may further comprise administering the modified BoNT/A to further muscles affected by said blepharospasm in accordance with the following dosage regimen:
(i) up to two unit doses (preferably one unit dose) to a corrugator muscle, or one unit dose to a procerus muscle; and
(ii) up to five unit doses (preferably one unit dose) to a frontalis muscle.

In other words, in any aspect or embodiment of the invention directed to treatment of blepharospasm, the invention may further comprise administering the modified BoNT/A to further muscles affected by said blepharospasm in accordance with the following dosage regimen:
(i) up to two unit doses (preferably one unit dose) per corrugator muscle (e.g. that is affected by said blepharospasm), or one unit dose to a procerus muscle; and
(ii) up to five unit doses (preferably one unit dose) to a frontalis muscle.

In any aspect or embodiment of the invention directed to treatment of blepharospasm, the invention may further comprise administering one or more unit dose of the modified BoNT/A to one or more further muscles affected by said blepharospasm in accordance with the following dosage regimen:
(i) one unit dose to a corrugator muscle;
(ii) two unit doses (e.g. one unit dose per affected eye) to a frontalis muscle; and/or
(iii) one unit dose to a procerus muscle.

In other words, in any aspect or embodiment of the invention directed to treatment of blepharospasm, the invention may further comprise administering one or more unit dose of the modified BoNT/A to one or more further muscles affected by said blepharospasm in accordance with the following dosage regimen:
(i) one unit dose per corrugator muscle (e.g. that is affected by said blepharospasm);
(ii) two unit doses (e.g. one unit dose per affected eye) to a frontalis muscle; and/or
(iii) one unit dose to a procerus muscle.

In any aspect or embodiment of the invention directed to treatment of blepharospasm, the invention may further comprise administering one or more unit dose of the modified BoNT/A to one or more further muscles affected by said blepharospasm in accordance with the following dosage regimen:
(i) one unit dose to a corrugator muscle, or one unit dose to a procerus muscle; and/or
(ii) two unit doses (doses (e.g. one unit dose per affected eye) to a frontalis muscle.

In other words, in any aspect or embodiment of the invention directed to treatment of blepharospasm, the invention may further comprise administering one or more unit dose of the modified BoNT/A to one or more further muscles affected by said blepharospasm in accordance with the following dosage regimen:
(i) one unit dose per corrugator muscle (e.g. that is affected by said blepharospasm), or one unit dose to a procerus muscle; and/or
(ii) two unit doses to a frontalis muscle.

In any aspect or embodiment of the invention directed to treatment of blepharospasm, the invention may further comprise administering the modified BoNT/A to further muscles affected by said blepharospasm in accordance with the following dosage regimen:
(i) one unit dose to a corrugator muscle;
(ii) two unit doses (e.g. one unit dose per affected eye) to a frontalis muscle; and
(iii) one unit dose to a procerus muscle.

In other words, in any aspect or embodiment of the invention directed to treatment of blepharospasm, the invention may further comprise administering the modified BoNT/A to further muscles affected by said blepharospasm in accordance with the following dosage regimen:
(i) one unit dose per corrugator muscle (e.g. that is affected by said blepharospasm);
(ii) two unit doses (e.g. one unit dose per affected eye) to a frontalis muscle; and
(iii) one unit dose to a procerus muscle.

In any aspect or embodiment of the invention directed to treatment of blepharospasm, the invention may further comprise administering the modified BoNT/A to further muscles affected by said blepharospasm in accordance with the following dosage regimen:
(i) one unit dose to a corrugator muscle, or one unit dose to a procerus muscle; and
(ii) two unit doses (doses (e.g. one unit dose per affected eye) to a frontalis muscle.

In other words, in any aspect or embodiment of the invention directed to treatment of blepharospasm, the invention may further comprise administering the modified BoNT/A to further muscles affected by said blepharospasm in accordance with the following dosage regimen:
(i) one unit dose per corrugator muscle (e.g. that is affected by said blepharospasm), or one unit dose to a procerus muscle; and
(ii) two unit doses to a frontalis muscle.

In any aspect or embodiment of the invention directed to treatment of blepharospasm, the invention may further comprise administering one unit dose to an orbicularis oris upper muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (viii) one unit dose to a buccinator muscle; (ix) up to two unit doses (preferably one unit dose) to a masseter muscle; (x) one unit dose to a procerus muscle; (x). one unit dose to a nasalis muscle; and/or (xii) one unit dose to a levator palpebrae superiori muscle.

In any aspect or embodiment of the invention directed to treatment of blepharospasm, the invention may further comprise administering one unit dose to an orbicularis oris lower muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris upper muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (viii) one unit dose to a buccinator muscle; (ix) up to two unit doses (preferably one unit dose) to a masseter muscle; (x) one unit dose to a procerus muscle; (x) one unit dose to a nasalis muscle; and/or (xii) one unit dose to a levator palpebrae superiori muscle.

In any aspect or embodiment of the invention directed to treatment of blepharospasm, the invention may further comprise administering one unit dose to a zygomaticus major muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to an orbicularis oris upper muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (viii) one unit dose to a buccinator muscle; (ix) up to two unit doses (preferably one unit dose) to a masseter muscle; (x) one unit dose to a procerus muscle; (x) one unit dose to a nasalis muscle; and/or (xii) one unit dose to a levator palpebrae superiori muscle.

In any aspect or embodiment of the invention directed to treatment of blepharospasm, the invention may further comprise administering one unit dose to a zygomaticus minor muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to a zygomaticus major muscle; (iii) one unit dose to an orbicularis oris upper muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (viii) one unit dose to a buccinator muscle; (ix) up to two unit doses (preferably one unit dose) to a masseter muscle; (x) one unit dose to a procerus muscle; (x). one unit dose to a nasalis muscle; and/or (xii) one unit dose to a levator palpebrae superiori muscle.

In any aspect or embodiment of the invention directed to treatment of blepharospasm, the invention may further comprise administering up to five unit doses (preferably one unit dose; more preferably two unit doses; most preferably three unit doses) to a frontalis muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to a zygomaticus major muscle; (iii) one unit dose to a zygomaticus minor muscle; (iv) one unit dose to an orbicularis oris upper muscle; (v) one unit dose to a mentalis muscle; (vi) one unit dose to a platysma muscle; (vii) up to two unit doses (preferably one unit dose) to a corrugator muscle; (viii) one unit dose to a buccinator muscle; (ix) up to two unit doses (preferably one unit dose) to a masseter muscle; (x) one unit dose to a procerus muscle; (x).
one unit dose to a nasalis muscle; and/or (xii) one unit dose to a levator palpebrae superiori muscle.

In any aspect or embodiment of the invention directed to treatment of blepharospasm, the invention may further comprise administering one unit dose to a mentalis muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to a zygomaticus major muscle; (iii) one unit dose to a zygomaticus minor muscle; (iv) up to five unit doses (preferably one unit dose) to a frontalis muscle; (v) one unit dose to an orbicularis oris upper muscle; (vi) one unit dose to a platysma muscle; (vii) up to two unit doses (preferably one unit dose) to a corrugator muscle; (viii) one unit dose to a buccinator muscle; (ix) up to two unit doses (preferably one unit dose) to a masseter muscle; (x) one unit dose to a procerus muscle; (x). one unit dose to a nasalis muscle; and/or (xii) one unit dose to a levator palpebrae superiori muscle.

In any aspect or embodiment of the invention directed to treatment of blepharospasm, the invention may further comprise administering one unit dose to a platysma muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to an orbicularis oris upper muscle; (vii) up to two unit doses (preferably one unit dose) to a corrugator muscle; (viii) one unit dose to a buccinator muscle; (ix) up to two unit doses (preferably one unit dose) to a masseter muscle; (x) one unit dose to a procerus muscle; (x) one unit dose to a nasalis muscle; and/or (xii) one unit dose to a levator palpebrae superiori muscle.

In any aspect or embodiment of the invention directed to treatment of blepharospasm, the invention may further comprise administering up to two unit doses (preferably one unit dose) to a corrugator muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) one unit dose to an orbicularis oris upper muscle; (viii) one unit dose to a buccinator muscle; (ix) up to two unit doses (preferably one unit dose) to a masseter muscle; (x) one unit dose to a procerus muscle; (x). one unit dose to a nasalis muscle; and/or (xii) one unit dose to a levator palpebrae superiori muscle.

In any aspect or embodiment of the invention directed to treatment of blepharospasm, the invention may further comprise administering one unit dose to a buccinator muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (viii one unit dose to an orbicularis oris upper muscle; (ix) up to two unit doses (preferably one unit dose) to a masseter muscle; (x) one unit dose to a procerus muscle; (x). one unit dose to a nasalis muscle; and/or (xii) one unit dose to a levator palpebrae superiori muscle.

In any aspect or embodiment of the invention directed to treatment of blepharospasm, the invention may further comprise administering one unit dose to up to two unit doses (preferably one unit dose) to a masseter muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (viii) one unit dose to a buccinator muscle; (ix) one unit dose to an orbicularis oris upper muscle; (x) one unit dose to a procerus muscle; (x) one unit dose to a nasalis muscle; and/or (xii) one unit dose to a levator palpebrae superiori muscle.

In any aspect or embodiment of the invention directed to treatment of blepharospasm, the invention may further comprise administering one unit dose to a procerus muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (viii) one unit dose to a buccinator muscle; (ix) up to two unit doses (preferably one unit dose) to a masseter muscle; (x) one unit dose to an orbicularis oris upper muscle; (x) one unit dose to a nasalis muscle; and/or (xii) one unit dose to a levator palpebrae superiori muscle.

In any aspect or embodiment of the invention directed to treatment of blepharospasm, the invention may further comprise administering one unit dose to a nasalis muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (viii) one unit dose to a buccinator muscle; (ix) up to two unit doses (preferably one unit dose) to a masseter muscle; (x) one unit dose to a procerus muscle; (x) one unit dose to an orbicularis oris upper muscle; and/or (xii) one unit dose to a levator palpebrae superiori muscle.

In any aspect or embodiment of the invention directed to treatment of blepharospasm, the invention may further comprise administering one unit dose to a levator palpebrae superiori muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said blepharospasm in accordance with the following dosage regimen:
(i) one unit dose to an orbicularis oris lower muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (viii) one unit dose to a buccinator muscle; (ix) up to two unit doses (preferably one unit dose) to a masseter muscle; (x) one unit dose to a procerus muscle; (x) one unit dose to a nasalis muscle; and/or (xii) one unit dose to an orbicularis oris upper muscle.

As outlined above, in aspects of the invention directed to treatment of typical hemifacial spasm, the invention may comprise administering one or more unit dose of the modified BoNT/A to one or more further muscles affected by said typical hemifacial spasm in accordance with the following dosage regimen: (i) one unit dose to an orbicularis oris upper muscle; (ii) one unit dose to an orbicularis oris lower muscle; (iii) one unit dose to a zygomaticus major muscle; (iv) one unit dose to a zygomaticus minor muscle; (v) up to five unit doses (preferably one unit dose) to a frontalis muscle; (vi) one unit dose to a mentalis muscle; (vii) one unit dose to a platysma muscle; (viii) up to two unit doses (preferably one unit dose) to a corrugator muscle; (ix) one unit dose to a buccinator muscle; (x) up to two unit doses (preferably one unit dose) to a masseter muscle; (xi) one unit dose to a procerus muscle; (xii) one unit dose to a nasalis muscle; and/or (xiii) one unit dose to a levator palpebrae superiori muscle.

In any aspect or embodiment of the invention directed to treatment of typical hemifacial spasm, the invention may further comprise administering (i) one unit dose to an orbicularis oris upper muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(ii) one unit dose to an orbicularis oris lower muscle; (iii) one unit dose to a zygomaticus major muscle; (iv) one unit dose to a zygomaticus minor muscle; (v) up to five unit doses (preferably one unit dose) to a frontalis muscle; (vi) one unit dose to a mentalis muscle; (vii) one unit dose to a platysma muscle; (viii) up to two unit doses (preferably one unit dose) to a corrugator muscle; (ix) one unit dose to a buccinator muscle; (x) up to two unit doses (preferably one unit dose) to a masseter muscle; (xi) one unit dose to a procerus muscle; (xii) one unit dose to a nasalis muscle; and/or (xiii) one unit dose to a levator palpebrae superiori muscle.

In any aspect or embodiment of the invention directed to treatment of typical hemifacial spasm, the invention may further comprise: administering one unit dose to an orbicularis oris lower muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (iix) one unit dose to a buccinator muscle; (ix) up to two unit doses (preferably one unit dose) to a masseter muscle; (x) one unit dose to a procerus muscle; (xi) one unit dose to a nasalis muscle; and/or (xii) one unit dose to a levator palpebrae superiori muscle.

In any aspect or embodiment of the invention directed to treatment of typical hemifacial spasm, the invention may further comprise: administering one unit dose to a zygomaticus major muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus minor muscle; (iii) an orbicularis oris lower muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (iix) one unit dose to a buccinator muscle; (ix) up to two unit doses (preferably one unit dose) to a masseter muscle; (x) one unit dose to a procerus muscle; (xi) one unit dose to a nasalis muscle; and/or (xii) one unit dose to a levator palpebrae superiori muscle.

In any aspect or embodiment of the invention directed to treatment of typical hemifacial spasm, the invention may further comprise: administering one unit dose to a zygomaticus minor muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus major muscle; (iii) one unit dose to an orbicularis oris lower muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (iix) one unit dose to a buccinator muscle; (ix) up to two unit doses (preferably one unit dose) to a masseter muscle; (x) one unit dose to a procerus muscle; (xi) one unit dose to a nasalis muscle; and/or (xii) one unit dose to a levator palpebrae superiori muscle.

In any aspect or embodiment of the invention directed to treatment of typical hemifacial spasm, the invention may further comprise: administering up to five unit doses (preferably one unit dose) to a frontalis muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus major muscle; (iii) one unit dose to a zygomaticus minor muscle; (iv) one unit dose to an orbicularis oris lower muscle; (v) one unit dose to a mentalis muscle; (vi) one unit dose to a platysma muscle; (vii) up to two unit doses (preferably one unit dose) to a corrugator muscle; (iix) one unit dose to a buccinator muscle; (ix) up to two unit doses (preferably one unit dose) to a masseter muscle; (x) one unit dose to a procerus muscle; (xi) one unit dose to a nasalis muscle; and/or (xii) one unit dose to a levator palpebrae superiori muscle.

In any aspect or embodiment of the invention directed to treatment of typical hemifacial spasm, the invention may further comprise: administering one unit dose to a mentalis muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus major muscle; (iii) one unit dose to a zygomaticus minor muscle; (iv) up to five unit doses (preferably one unit dose) to a frontalis muscle; (v) one unit dose to an orbicularis oris lower muscle; (vi) one unit dose to a platysma muscle; (vii) up to two unit doses (preferably one unit dose) to a corrugator muscle; (iix) one unit dose to a buccinator muscle; (ix) up to two unit doses (preferably one unit dose) to a masseter muscle; (x) one unit dose to a procerus muscle; (xi) one unit dose to a nasalis muscle; and/or (xii) one unit dose to a levator palpebrae superiori muscle.

In any aspect or embodiment of the invention directed to treatment of typical hemifacial spasm, the invention may further comprise: administering one unit dose to a platysma muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to an orbicularis oris lower muscle; (vii) up to two unit doses (preferably one unit dose) to a corrugator muscle; (iix) one unit dose to a buccinator muscle; (ix) up to two unit doses (preferably one unit dose) to a masseter muscle; (x) one unit dose to a procerus muscle; (xi) one unit dose to a nasalis muscle; and/or (xii) one unit dose to a levator palpebrae superiori muscle.

In any aspect or embodiment of the invention directed to treatment of typical hemifacial spasm, the invention may further comprise: administering up to two unit doses (preferably one unit dose) to a corrugator muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) one unit dose to an orbicularis oris lower muscle; (iix) one unit dose to a buccinator muscle; (ix) up to two unit doses (preferably one unit dose) to a masseter muscle; (x) one unit dose to a procerus muscle; (xi) one unit dose to a nasalis muscle; and/or (xii) one unit dose to a levator palpebrae superiori muscle.

In any aspect or embodiment of the invention directed to treatment of typical hemifacial spasm, the invention may further comprise: administering one unit dose to a buccinator muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (iix) one unit dose to an orbicularis oris lower muscle; (ix) up to two unit doses (preferably one unit dose) to a masseter muscle; (x) one unit dose to a procerus muscle; (xi) one unit dose to a nasalis muscle; and/or (xii) one unit dose to a levator palpebrae superiori muscle.

In any aspect or embodiment of the invention directed to treatment of typical hemifacial spasm, the invention may further comprise: administering up to two unit doses (preferably one unit dose) to a masseter muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (iix) one unit dose to a buccinator muscle; (ix) one unit dose to an orbicularis oris lower muscle; (x) one unit dose to a procerus muscle; (xi) one unit dose to a nasalis muscle; and/or (xii) one unit dose to a levator palpebrae superiori muscle.

In any aspect or embodiment of the invention directed to treatment of typical hemifacial spasm, the invention may further comprise: administering one unit dose to a procerus muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (iix) one unit dose to a buccinator muscle; (ix) up to two unit doses (preferably one unit dose) to a masseter muscle; (x) one unit dose to an orbicularis oris lower muscle; (xi) one unit dose to a nasalis muscle; and/or (xii) one unit dose to a levator palpebrae superiori muscle.

In any aspect or embodiment of the invention directed to treatment of typical hemifacial spasm, the invention may further comprise: administering one unit dose to a nasalis muscle affected by said typical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (iix) one unit dose to a buccinator muscle; (ix) up to two unit doses (preferably one unit dose) to a masseter muscle; (x) one unit dose to a procerus muscle; (xi) one unit dose to an orbicularis oris lower muscle; and/or (xii) one unit dose to a levator palpebrae superiori muscle.

In any aspect or embodiment of the invention directed to treatment of typical hemifacial spasm, the invention may further comprise: administering one unit dose to a levator palpebrae superiori muscle affected by said typical hemifacial spasm; and optionally one or more unit dose of the modified BoNT/A to one or more (e.g. 2 or more, 3 or more, 4 or more, or more, 6 or more, 7 or more, 8 or more, 9 or more, 10 or more, 11 or more, or 12 or more) further muscles affected by said typical hemifacial spasm in accordance with the following dosage regimen:
(i) one unit dose to an orbicularis oris upper muscle; (ii) one unit dose to a zygomaticus major muscle; (iii) one unit dose to a zygomaticus minor muscle; (iv) up to five unit doses (preferably one unit dose) to a frontalis muscle; (v) one unit dose to a mentalis muscle; (vi) one unit dose to a platysma muscle; (vii) up to two unit doses (preferably one unit dose) to a corrugator muscle; (iix) one unit dose to a buccinator muscle; (ix) up to two unit doses (preferably one unit dose) to a masseter muscle; (x) one unit dose to a procerus muscle; (xi) one unit dose to a nasalis muscle; and/or (xii) one unit dose to an orbicularis oris lower muscle.

As outlined above, in aspects of the invention directed to treatment of atypical hemifacial spasm, the invention may comprise administering one or more unit dose of the modified BoNT/A to one or more further muscles affected by said atypical hemifacial spasm in accordance with the following dosage regimen:
(i) one unit dose to a zygomaticus major muscle; (ii) one unit dose to a zygomaticus minor muscle; (iii) up to five unit doses (preferably one unit dose) to a frontalis muscle; (iv) one unit dose to a mentalis muscle; (v) one unit dose to a platysma muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a procerus muscle; (x) one unit dose to a nasalis muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) one unit dose to a zygomaticus major muscle affected by said atypical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a mentalis muscle; (v) one unit dose to a platysma muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a procerus muscle; (x) one unit dose to a nasalis muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) one unit dose to a zygomaticus minor muscle affected by said atypical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus major muscle; (iii) up to five unit doses (preferably one unit dose) to a frontalis muscle; (iv) one unit dose to a mentalis muscle; (v) one unit dose to a platysma muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a procerus muscle; (x) one unit dose to a nasalis muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) up to five unit doses (preferably one unit dose) to a frontalis muscle; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) one unit dose to a zygomaticus major muscle; (iv) one unit dose to a mentalis muscle; (v) one unit dose to a platysma muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a procerus muscle; (x) one unit dose to a nasalis muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) one unit dose to a mentalis muscle affected by said atypical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a zygomaticus major muscle; (v) one unit dose to a platysma muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a procerus muscle; (x) one unit dose to a nasalis muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) one unit dose to a platysma muscle affected by said atypical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a mentalis muscle; (v) one unit dose to a zygomaticus major muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a procerus muscle; (x) one unit dose to a nasalis muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) up to two unit doses (preferably one unit dose) to a corrugator muscle affected by said atypical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a mentalis muscle; (v) one unit dose to a platysma muscle; (vi) one unit dose to a zygomaticus major muscle; (vii) one unit dose to a buccinator muscle; (viii) up to two unit doses (preferably one unit dose) to a masseter muscle; (ix) one unit dose to a procerus muscle; (x) one unit dose to a nasalis muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) one unit dose to a buccinator muscle affected by said atypical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a mentalis muscle; (v) one unit dose to a platysma muscle; (vi) up to two unit doses (preferably one unit dose) to a corrugator muscle; (vii) one unit dose to a zygomaticus major muscle; (viii) up to two unit doses (preferably one unit dose) to a masseter muscle; (ix) one unit dose to a procerus muscle; (x) one unit dose to a nasalis muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) up to two unit doses (preferably one unit dose) to a masseter muscle affected by said atypical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a mentalis muscle; (v) one unit dose to a platysma muscle; (vi) up to two unit doses (preferably one unit dose) to a corrugator muscle; (vii) one unit dose to a buccinator muscle; (viii) one unit dose to a zygomaticus major muscle; (ix) one unit dose to a procerus muscle; (x) one unit dose to a nasalis muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) one unit dose to a procerus muscle affected by said atypical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a mentalis muscle; (v) one unit dose to a platysma muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a zygomaticus major muscle; (x) one unit dose to a nasalis muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) one unit dose to a nasalis muscle affected by said atypical hemifacial spasm; and optionally one or more unit dose of the modified BoNT/A to one or more (e.g. 2 or more, 3 or more, 4 or more, 5 or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a mentalis muscle; (v) one unit dose to a platysma muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a procerus muscle; (x) one unit dose to a zygomaticus major muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) one unit dose to a lateral upper orbicularis oculi muscle affected by said atypical hemifacial spasm; and optionally one or more unit dose of the modified BoNT/A to one or more (e.g. 2 or more, 3 or more, 4 or more, or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a mentalis muscle; (v) one unit dose to a platysma muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a procerus muscle; (x) one unit dose to a nasalis muscle; (xi) one unit dose to a zygomaticus major muscle; (xii) one unit dose to a medial upper orbicularis oculi muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) one unit dose to a medial upper orbicularis oculi muscle affected by said atypical hemifacial spasm; and optionally one or more unit dose of the modified BoNT/A to one or more (e.g. 2 or more, 3 or more, 4 or more, or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a mentalis muscle; (v) one unit dose to a platysma muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a procerus muscle; (x) one unit dose to a nasalis muscle; (xi) one unit dose to a lateral upper orbicularis oculi muscle; (xii) one unit dose to a zygomaticus major muscle; (xiii) one unit dose to a lateral lower orbicularis oculi muscle; and/or (xiv) one unit dose to a levator palpebrae superiori muscle.

In any aspect or embodiment of the invention directed to treatment of atypical hemifacial spasm, the invention may further comprise: administering (i) one unit dose to a lateral lower orbicularis oculi muscle affected by said atypical hemifacial spasm; and optionally one or more unit dose of the modified BoNT/A to one or more (e.g. 2 or more, 3 or more, 4 or more, or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a mentalis muscle; (v) one unit dose to a platysma muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a procerus muscle; (x) one unit dose to a nasalis muscle; (xi) one unit dose to a lateral upper orbicularis oculi muscle; (xii) one unit dose to a medial upper orbicularis oculi muscle; (xiii) one unit dose to a zygomaticus major muscle; and/or (xiv) one unit dose to a levator palpebrae superiori muscle.

In any aspect or embodiment of the invention directed to treatment of atypical hemifacial spasm, the invention may further comprise: administering (i) one unit dose to a levator palpebrae superiori muscle affected by said atypical hemifacial spasm; and optionally one or more unit dose of the modified BoNT/A to one or more (e.g. 2 or more, 3 or more, 4 or more, or more, 6 or more, 7 or more, 8 or more, 9 or more, 10 or more, 11 or more, 12 or more, or 13 or more) further muscles affected by said atypical hemifacial spasm in accordance with the following dosage regimen:
(ii) one unit dose to a zygomaticus minor muscle; (iii) up to five unit doses (preferably one unit dose) to a frontalis muscle; (iv) one unit dose to a mentalis muscle; (v) one unit dose to a platysma muscle; (vi) up to two unit doses (preferably one unit dose) to a corrugator muscle; (vii) one unit dose to a buccinator muscle; (viii) up to two unit doses (preferably one unit dose) to a masseter muscle; (ix) one unit dose to a procerus muscle; (x) one unit dose to a nasalis muscle; (xi) one unit dose to a lateral upper orbicularis oculi muscle; (xii) one unit dose to a medial upper orbicularis oculi muscle; (xiii) one unit dose to a lateral lower orbicularis oculi muscle; and/or (xiv) one unit dose to a zygomaticus major muscle.

A modified BoNT/A may be administered to a muscle and/or site thereof according to the invention by any suitable means.

As an alternative to intramuscular injection, a modified BoNT/A may be administered subcutaneously, e.g. by subcutaneous injection. Said subcutaneous injection may include injection medially and/or laterally into the junction between the preseptal and orbital parts of the upper and/or lower orbicularis oculi muscles, as required.

Most preferably, a modified BoNT/A is administered intramuscularly, e.g. by intramuscular injection.

Electromyographic control/guidance may be employed to assist in administering a modified BoNT/A in accordance with the invention.

The term “a unit dose” may embrace more than one unit dose. For example, the term “a unit dose” may mean up to two unit doses, up to three unit doses, up to four unit doses or up to five unit doses. The term “a unit dose” may also refer to a single unit dose.

A single unit dose may be administered to an affected muscle at one or more injection sites. Where a single unit dose is administered at more than one injection site, the unit dose may be divided (equally or unequally) between two or more injection sites. However, it is preferred that a single unit dose is administered per injection site.

The term “a single unit dose is administered” means substantially all of a single unit dose is administered. For example, a residual amount (e.g. up to 1%, 0.1% or 0.01%) of the unit dose may remain in a vial in which the modified BoNT/A has been reconstituted. However, preferably all of a single unit dose is administered (e.g. at one or more injection sites, preferably per injection site). This definition applies analogously to administration of two unit doses, three unit doses, etc.

Potency of a modified BoNT/A for use according to the invention may be determined by a mouse LD₅₀ assay according to standard techniques. In said assay, 1 Unit is defined as an amount of the modified BoNT/A that corresponds to the calculated median lethal dose (LD₅₀) in mice. Preferably, the calculated median lethal intraperitoneal dose in mice

Where a modified BoNT/A for use in the invention is modified BoNT/A comprising a BoNT/A light-chain and translocation domain, and a BoNT/B receptor binding domain (H_{C} domain), an amount of a modified BoNT/A that corresponds to 1 Unit in said assay is preferably 24.04 pg.

The term “up to” when used in reference to a value (e.g. up to 82,500 pg) means up to and including the value recited. Thus, as an example, reference to administering “up to 82,500 pg” of modified BoNT/A encompasses administration of 82,500 pg of modified BoNT/A as well as administration of less than 82,500 pg of modified BoNT/A.

A unit dose may be expressed in terms of an amount of modified BoNT/A, in Units of modified BoNT/A, or a combination thereof.

The total number of unit doses administered may be up to 20, 15, 10, 5 or 3. The total number of unit doses administered may be at least 3, 5, 10, or 15. The total number of unit doses administered may be 3-20, 4-16, or 5-12. In one embodiment, 5 unit doses are administered. In one embodiment, 6 unit doses are administered. In one embodiment, 10 unit doses are administered. In one embodiment, 12 unit doses are administered. In one embodiment, 15 doses are administered. Administration of 12 units doses in total is preferred, particularly in the treatment of blepharospasm (more particularly bilateral blepharospasm).

The modified BoNT/A may be administered by intramuscular injection to total of six, seven, eight, nine, ten, eleven or twelve sites of the of a first eye of the subject. The modified BoNT/A may be administered by intramuscular injection to total of six, seven, eight, nine, ten, or eleven sites of the of a first eye of the subject. Additionally or alternatively, the modified BoNT/A may be administered by intramuscular injection to total of six, seven, eight, nine, ten, eleven or twelve sites of the of a second eye of the subject. The modified BoNT/A may be administered by intramuscular injection to total of six, seven, eight, nine, ten, or eleven sites of the of a second eye of the subject.

In a preferable embodiment, the modified BoNT/A is administered by intramuscular injection to total of six sites of the of a first eye of the subject. Additionally or alternatively, the modified BoNT/A may preferably be administered by intramuscular injection to total of six sites of the of a second eye of the subject.

In the case of treating blepharospasm (e.g. preferably bilateral blepharospasm), it is preferred that up to 12 unit doses be administered across the following sites:
two unit doses to the lateral upper orbicularis oculi muscle of an affected eye (e.g. two unit doses into said site of each eye for a total of four unit doses);
one unit dose to the medial upper orbicularis oculi muscle of an affected eye (e.g. one unit dose into said site of each eye for a total of two unit doses);
one unit dose to the lateral lower orbicularis oculi muscle of an affected eye (e.g. one unit dose into said site of each eye for a total of two unit doses);
one unit dose to a procerus muscle;
one unit dose to a corrugator muscle proximal to an affected (e.g. one unit dose into said site of each eye for a total of two unit doses); and
one unit dose into a frontalis muscle proximal to an affected eye (e.g. one unit dose into said site of each eye for a total of two unit doses).

For example, the treatment may comprise a total of 12 unit dose injections, across the above described sites. It is preferred that 12 unit dose injections (particularly for blepharospasm, more particularly of the bilateral type) be administered, across the above described sites.

In the case of treating blepharospasm that is unilateral, it is preferred that up to 6 unit doses be administered across the following sites:
two unit doses to the lateral upper orbicularis oculi muscle of an affected eye
one unit dose to the medial upper orbicularis oculi muscle of an affected eye
one unit dose to the lateral lower orbicularis oculi muscle of an affected eye
one unit dose to a procerus muscle;
one unit dose to a corrugator muscle proximal to an affected; and
one unit dose into a frontalis muscle proximal to an affected eye.

For example, the treatment may comprise a total of 6 unit dose injections, across the above described sites.

Optionally, a unit dose may be administered to only one (but not both) selected from the procerus and a corrugator.

It is preferred that each of the following injection sites are encompassed by the above-described treatment regimens (e.g. which may be referred to as the “minimum” injection sites):
two unit doses to the lateral upper orbicularis oculi muscle of an affected eye (e.g. two unit doses into said site of each eye for a total of four unit doses);
one unit dose to the medial upper orbicularis oculi muscle of an affected eye (e.g. one unit dose into said site of each eye for a total of two unit doses); and
one unit dose to the lateral lower orbicularis oculi muscle of an affected eye (e.g. one unit dose into said site of each eye for a total of two unit doses).

Said ‘minimum’ injection sites may be sufficient to effect the treatment, such that a total of four unit doses per eye (e.g. a total of four unit doses in the case of unilateral blepharospasm) are administered. That being said, the treatment may be extended to the procerus and/or corrugator (preferably procerus or corrugator), adding an additional unit dose for the procerus and/or an additional unit dose per corrugator to the treatment regimen. Additionally or alternatively, the treatment may be extended to a frontalis of an affected eye, adding an additional unit dose (for the frontalis) per affected eye.

The skilled person will take into consideration when a subject has recently had (or is subsequently having) additional treatment with a clostridial neurotoxin (e.g. unmodified BoNT), e.g. as part of a cosmetic treatment or treatment for a different indication. Using techniques routine in the art, the skilled person will adapt the present treatment regimen accordingly. Preferably, the present invention excludes treatment with a further clostridial neurotoxin (e.g. BoNT).

A modified BoNT/A of the invention preferably has a longer duration of action when compared to unmodified BoNT/A (e.g. Dysport®), e.g. the action being improvement in one or more symptoms blepharospasm or hemifacial spams such as for instance at least 5%, 10%, 25%, or 50% improvement compared to said one or more symptoms pre-treatment. Said duration of action may be at least 1.25×, 1.5×, 1.75×, 2.0×, or 2.25× greater. The duration of action of modified BoNT/A may be between 6 and 9 months. For example, a duration of action may be at least: 4.5 months (from onset), 5.0 months, 5.5 months, 6 months, 6.5 months, 7.0 months, 7.5 months, 8.0 months, 8.5 months or 9.0 months. In particular embodiments, a duration of action may be greater than 9.0 months.

Treatment may be repeated at an appropriate time period following administration of modified BoNT/A. Given that the duration of action is approximately twice that of unmodified BoNT/A (e.g. Dysport®) there are suitably longer periods between subsequent administrations than when a subject is treated with unmodified BoNT/A (e.g. Dysport®). A subject may be re-administered a modified BoNT/A in accordance with the present invention at least 18, 20, 25 or 30 weeks following a previous administration. For example, a subject may be re-administered a modified BoNT/A in accordance with the present invention at least 18-45 weeks, preferably 20-35 weeks following a previous administration.

A “subject” as used herein may be a mammal, such as a human or other mammal. Preferably “subject” means a human subject. A “subject” is preferably an adult subject, i.e. a subject at least 18 years old. The terms “subject” and “patient” are used synonymously herein. Preferably, the subject has been diagnosed with a facial dystonia of the invention (blepharospasm, typical hemifacial spasm, or atypical hemifacial spasm).

A subject for treatment in accordance with the invention may be a subject that is unsuitable for treatment with an unmodified BoNT/A (e.g. of SEQ ID NO: 2). Said subject may be a subject that is resistant to treatment with an unmodified BoNT/A. Resistance may arise due to development of an immune response to a clostridial neurotoxin, including production of anti-clostridial neurotoxin antibodies, by a subject.

The term “treat” or “treating” as used herein encompasses prophylactic treatment (e.g. to prevent onset of a disorder) as well as corrective treatment (treatment of a subject already suffering from a disorder). Preferably “treat” or “treating” as used herein means corrective treatment. The term “treat” or “treating” as used herein refers to the disorder and/or a symptom thereof.

BoNT/A is one example of a clostridial neurotoxin produced by bacteria in the genus Clostridia. Other examples of such clostridial neurotoxins include those produced by C. tetani (TeNT) and by C. botulinum (BoNT) serotypes B-G and X (see WO 2018/009903 A2), as well as those produced by C. baratii and C. butyricum. Said neurotoxins are highly potent and specific and can poison neurons and other cells to which they are delivered. The clostridial toxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins (including BoNT/A) are synthesised as a single-chain polypeptide that is modified post-translationally by a proteolytic cleavage event to form two polypeptide chains joined together by a disulphide bond. Cleavage occurs at a specific cleavage site, often referred to as the activation site (e,g, activation loop), that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the HC domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins.

Accordingly, once delivered to a desired target cell, the non-cytotoxic protease is capable of inhibiting cellular secretion from the target cell. The L-chain proteases of clostridial toxins are non-cytotoxic proteases that cleave SNARE proteins.

In view of the ubiquitous nature of SNARE proteins, clostridial neurotoxins such as botulinum toxin have been successfully employed in a wide range of therapies.

For further details on the genetic basis of toxin production in Clostridium botulinum and C. tetani, see Henderson et al (1997) in The Clostridia: Molecular Biology and Pathogenesis, Academic press.

As discussed above, clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or HC domain) and an N-terminal translocation component (H_{N} domain).

Clostridial neurotoxin domains are described in more detail below.

Examples of L-chain reference sequences include:
Botulinum type A neurotoxin: amino acid residues 1-448
Botulinum type B neurotoxin: amino acid residues 1-440

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441

The translocation domain is a fragment of the H-chain of a clostridial neurotoxin approximately equivalent to the amino-terminal half of the H-chain, or the domain corresponding to that fragment in the intact H-chain.

Examples of reference translocation domains include:
Botulinum type A neurotoxin—amino acid residues (449-871)
Botulinum type B neurotoxin—amino acid residues (441-858)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-S858)

In the context of the present invention, a variety of BoNT/A H_{N} regions comprising a translocation domain can be useful in aspects of the present invention. The H_{N} regions from the heavy-chain of BoNT/A are approximately 410-430 amino acids in length and comprise a translocation domain. Research has shown that the entire length of a H_{N} region from a clostridial neurotoxin heavy-chain is not necessary for the translocating activity of the translocation domain. Thus, aspects of this embodiment can include BoNT/A H_{N} regions comprising a translocation domain having a length of, for example, at least 350 amino acids, at least 375 amino acids, at least 400 amino acids or at least 425 amino acids. Other aspects of this embodiment can include BoNT/A H_{N} regions comprising a translocation domain having a length of, for example, at most 350 amino acids, at most 375 amino acids, at most 400 amino acids or at most 425 amino acids.

The term H_{N} embraces naturally-occurring BoNT/A H_{N} portions, and modified BoNT/A H_{N} portions having amino acid sequences that do not occur in nature and/or synthetic amino acid residues. Preferably, said modified BoNT/A H_{N} portions still demonstrate the above-mentioned translocation function.

Examples of clostridial neurotoxin receptor binding domain (H_{C}) reference sequences include:
BoNT/A—N872-L1296
BoNT/B—E859-E1291

The ˜50 kDa HC domain of a clostridial neurotoxin (such as a BoNT) comprises two distinct structural features that are referred to as the H_{CC} and HC_{N} domains, each typically of ˜25 kDa. Amino acid residues involved in receptor binding are believed to be primarily located in the H_{CC} domain. The H_{C} domain of a native clostridial neurotoxin may comprise approximately 400-440 amino acid residues. This fact is confirmed by the following publications, each of which is herein incorporated in its entirety by reference thereto: Umland TC (1997) Nat. Struct. Biol. 4: 788-792; Herreros J (2000) Biochem. J. 347: 199-204; Halpern J (1993) J. Biol. Chem. 268: 15, pp. 11188-11192; Rummel A (2007) PNAS 104: 359-364; Lacey DB (1998) Nat. Struct. Biol. 5: 898-902; Knapp (1998) Am. Cryst. Assoc. Abstract Papers 25: 90; Swaminathan and Eswaramoorthy (2000) Nat. Struct. Biol. 7: 1751-1759; and Rummel A (2004) Mol. Microbiol. 51(3), 631-643.

Examples of (reference) H_{CN} domains include:
Botulinum type A neurotoxin—amino acid residues (872-1110)
Botulinum type B neurotoxin—amino acid residues (859-1097)

The above sequence positions may vary a little according to serotype/sub-type, and further examples of (reference) H_{CN} domains include:
Botulinum type A neurotoxin—amino acid residues (874-1110)
Botulinum type B neurotoxin—amino acid residues (861-1097)

Examples of (reference) H_{CC} domains include:
Botulinum type A neurotoxin—amino acid residues (Y1111-L1296)
Botulinum type B neurotoxin—amino acid residues (Y1098-E1291)

The L-chain and H_{N} domain (optionally including a complete or partial activation loop, e.g. a complete activation loop when the modified BoNT/A is in a single-chain form and a cleaved/partial activation loop when in a di-chain form) may be collectively referred to as an LH_{N} domain. The LH_{N} domain thus may not further comprise an H_{C} domain.

WO 2017/191315 A1 (which is incorporated herein by reference) teaches modified BoNT/As and methods for preparing and manufacturing the same. Thus, a modified BoNT/A comprising a botulinum neurotoxin A (BoNT/A) light-chain and translocation domain (BoNT/A H_{N}), and a BoNT/B receptor binding domain (H_{C} domain) for use in the present invention may be one taught in WO 2017/191315 A1.

The term “modified BoNT/A” or “chimeric clostridial neurotoxin” or “chimeric neurotoxin” as used herein means a neurotoxin comprising (preferably consisting of) a clostridial neurotoxin light-chain and translocation domain (H_{N} domain) from a first clostridial neurotoxin serotype and a receptor binding domain (H_{C} domain) originating from a second different clostridial neurotoxin serotype. Specifically, a modified BoNT/A for use in the invention comprises a botulinum neurotoxin A (BoNT/A) light-chain and translocation domain (H_{N} domain), and a BoNT/B receptor binding domain (H_{C} domain). The BoNT/A LH_{N} domain of the modified BoNT/A is covalently linked to the BoNT/B H_{C} domain. The modified BoNT/A of the invention may be referred to as a chimeric botulinum neurotoxin. Said modified BoNT/A is also referred to herein as “BoNT/AB”, “mrBoNT/AB” or a “BoNT/AB chimera”.

The L-chain and H_{N} domain (optionally including a complete or partial activation loop, e.g. a complete activation loop when the modified BoNT/A is in a single-chain form and a cleaved/partial activation loop when in a di-chain form) may be collectively referred to as an LH_{N} domain. The LH_{N} domain thus does not further comprise an H_{C} domain.

The modified BoNT/A may consist essentially of a botulinum neurotoxin A (BoNT/A) light-chain and translocation domain (H_{N} domain), and a BoNT/B receptor binding domain (H_{C} domain).

The term “consist(s) essentially of” as used in this context means that the modified BoNT/A does not further comprise one or more amino acid residues that confer additional functionality to the polypeptide, e.g. when administered to a subject. In other words, a polypeptide that “consists essentially of” a botulinum neurotoxin A (BoNT/A) light-chain and translocation domain (H_{N} domain), and a BoNT/B receptor binding domain (H_{C} domain) may further comprise one or more amino acid residues (to those of the botulinum neurotoxin A (BoNT/A) light-chain and translocation domain (H_{N} domain), and BoNT/B receptor binding domain (H_{C} domain)) but said one or more further amino acid residues do not confer additional functionality to the polypeptide, e.g. when administered to a subject. Additional functionality may include enzymatic activity, binding activity and/or any physiological activity whatsoever.

The modified BoNT/A may comprise non-clostridial neurotoxin sequences in addition to any clostridial neurotoxin sequences so long as the non-clostridial neurotoxin sequences do not disrupt the ability of the modified BoNT/A to achieve its therapeutic effect. Preferably, the non-clostridial neurotoxin sequence is not one having catalytic activity, e.g. enzymatic activity. In one embodiment the modified BoNT/A of the invention does not comprise a non-clostridial catalytically active domain. In one embodiment, a modified BoNT/A does not comprise a further catalytically active domain. In one embodiment, the non-clostridial sequence is not one that binds to a cellular receptor. In other words, in one embodiment, the non-clostridial sequence is not a ligand for a cellular receptor. A cellular receptor may be a proteinaceous cellular receptor, such as an integral membrane protein. Examples of cellular receptors can be found in the IUPHAR Guide to Pharmacology Database, version 2019.4, available at https://www.guidetopharmacology.org/download.jsp #db_reports. Non-clostridial neurotoxin sequences may include tags to aid in purification, such as His-tags. In one embodiment, a modified BoNT/A of the invention does not comprise a label or a site for adding a label, such as a sortase acceptor or donor site.

Preferably, a modified BoNT/A may consist of a botulinum neurotoxin A (BoNT/A) light-chain and translocation domain (H_{N} domain), and a BoNT/B receptor binding domain (H_{C} domain).

The modified BoNT/A comprises a light-chain that is capable of exhibiting non-cytotoxic protease activity and of cleaving a SNARE protein in the cytosol of a target neuron. Cell-based and in vivo assays may be used to determine if a clostridial neurotoxin comprising an L-chain and a functional cell binding and translocation domain has non-cytotoxic protease activity. Assays such as the Digit Abduction Score (DAS) assay, the dorsal root ganglia (DRG) assay, spinal cord neuron (SCN) assay, and mouse phrenic nerve hemidiaphragm (PNHD) assay are routine in the art. A suitable assay for determining non-cytotoxic protease activity may be one described in Aoki K R, Toxicon 39: 1815-1820; 2001 or Donald et al (2018), Pharmacol Res Perspect, e00446, 1-14, which are incorporated herein by reference.

When administered to a subject, a modified BoNT/A is preferably in its active di-chain form where the light-chain and heavy-chain are joined together by a disulphide bond. Where a BoNT/A (e.g. modified BoNT/A) is defined herein by way of a polypeptide sequence (SEQ ID NO), an L-chain portion of the sequence (SEQ ID NO) may constitute a first chain of the di-chain clostridial neurotoxin (e.g. di-chain modified BoNT/A) and the H_{N} and H_{C} domains together may constitute a second chain of the di-chain clostridial neurotoxin (e.g. di-chain modified BoNT/A), wherein the first and second chains are joined together by a di-sulphide bond. The skilled person will appreciate that a protease may cleave at one or more positions within the activation loop of the clostridial neurotoxin (e.g. modified BoNT/A), preferably at two positions within the activation loop. Where cleavage occurs at more than one position (preferably at two positions) within the activation loop, a small fragment of the C-terminal L-chain portion of the sequence may be absent from the di-chain clostridial neurotoxin sequence (e.g. di-chain modified BoNT/A). In view of this, the sequence of the di-chain clostridial neurotoxin (e.g. di-chain modified BoNT/A) may be slightly different to that of the corresponding single-chain clostridial neurotoxin (e.g. single-chain modified BoNT/A). The small fragment may be 1-15 amino acids. In particular, in one embodiment, when Lys-C is used to convert a single-chain modified BoNT/A into a di-chain modified BoNT/A, the small fragment of the C-terminal L-chain portion of the sequence that is absent may be SEQ ID NO: 9 or 10.

Most preferably, a modified BoNT/A for use in the invention may comprise a BoNT/A light-chain and translocation domain (a BoNT/A LH_{N} domain), and a BoNT/B H_{C} domain. The BoNT/A LH_{N} domain is covalently linked to the BoNT/B H_{C} domain. Said modified BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

An example of a BoNT/A polypeptide sequence is provided as SEQ ID NO: 2 (such as a di-chain form of SEQ ID NO: 2).

An example of a BoNT/B polypeptide sequence is provided as SEQ ID NO: 8 (UniProt accession number B11NP5).

Reference herein to the “first amino acid residue of the 3₁₀ helix separating the LH_{N} and He domains of BoNT/A” means the N-terminal residue of the 3₁₀ helix separating the LH_{N} and He domains.

Reference herein to the “second amino acid residue of the 3₁₀ helix separating the LH_{N} and He domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3->i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an α-helix at its N-terminus (i.e. at the C-terminal part of the LH_{N} domain) and by a β-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publicly available crystal structures of botulinum neurotoxins, for example 3BTA (http://www.rcsb.org/pdb/explore/explore.do?structureld=3BTA) and 1EPW (http://www.rcsb.org/pdb/explore/explore.do?structureld=1EPW) for botulinum neurotoxins A1 and 1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular, the region around the “H_{N}/H_{CN}” junction may be structurally highly conserved which renders it an ideal region to superimpose different serotypes.

For example, the following methodology may be used to determine the sequence of this 3₁₀ helix in other neurotoxins:
1. The structural homology modelling tool LOOP (http://loopp.org) may be used to obtain a predicted structure of other BoNT serotypes based on the BoNT/A1 crystal structure (3BTA.pdb);
2. The structural (pdb) files thus obtained may be edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) may be used to superpose each serotype onto the 3BTA.pdb structure;
4. The superposed pdb files may be inspected to locate the 3₁₀ helix at the start of the H_{C} domain of BoNT/A1, and corresponding residues in the other serotype may then identified.
5. The other BoNT serotype sequences may be aligned with Clustal Omega in order to check that corresponding residues were correct.

Examples of LH_{N}, H_{C} and 3₁₀ helix domains determined by this method are presented below:

| | Accession Number | | | |
|---|---|---|---|---|
| | (Plus Sequence | | | |
| | Version after | | | |
| Neurotoxin | Decimal) | LH_{N} | H_{C} | 3₁₀ helix |
| BoNT/A1 | A5HZZ9.1 | 1-872 | 873-1296 | ⁸⁷²NIINTS⁸⁷⁷ |
| (SEQ ID | | | | |
| NO: 2) | | | | |
| BoNT/A2 | X73423.3 | 1-872 | 873-1296 | ⁸⁷²NIVNTS⁸⁷⁷ |
| BoNT/A3 | DQ185900.1 (aka | 1-872 | 873-1292 | ⁸⁷²NIVNTS⁸⁷⁷ |
| | Q3LRX9.1) | | | |
| BoNT/A4 | EU341307.1 (aka | 1-872 | 873-1296 | ⁸⁷²NITNAS⁸⁷⁷ |
| | Q3LRX8.1) | | | |
| BoNT/A5 | EU679004.1 (aka | 1-872 | 873-1296 | ⁸⁷²NIINTS⁸⁷⁷ |
| | C1IPK2.1) | | | |
| BoNT/A6 | FJ981696.1 | 1-872 | 873-1296 | ⁸⁷²NIINTS⁸⁷⁷ |
| BoNT/A7 | JQ954969.1 (aka | 1-872 | 873-1296 | ⁸⁷²NIINTS⁸⁷⁷ |
| | K4LN57.1) | | | |
| BoNT/A8 | KM233166.1 | 1-872 | 873-1297 | ⁸⁷²NITNTS⁸⁷⁷ |
| BoNT/B1 | B1INP5.1 | 1-859 | 860-1291 | ⁸⁵⁹EILNNI⁸⁶⁴ |
| (SEQ ID | | | | |
| NO: 8) | | | | |
| BoNT/B2 | AB084152.1 (aka | 1-859 | 860-1291 | ⁸⁵⁹EILNNI⁸⁶⁴ |
| | Q8GR96.1) | | | |
| BoNT/B3 | EF028400.1 (aka | 1-859 | 860-1291 | ⁸⁵⁹EILNNI⁸⁶⁴ |
| | A2I2S2.1) | | | |
| BoNT/B4 | EF051570.1 (aka | 1-859 | 860-1291 | ⁸⁵⁹EILNNI⁸⁶⁴ |
| | A2I2W0.1) | | | |
| BoNT/B5 | EF033130.1 (aka | 1-859 | 860-1291 | ⁸⁵⁹DILNNI⁸⁶⁴ |
| | A2I2U6.1) | | | |
| BoNT/B6 | AB302852.1 (aka | 1-859 | 860-1291 | ⁸⁵⁹EILNNI⁸⁶⁴ |
| | A8R089.1) | | | |
| BoNT/B7 | JQ354985.1 (aka | 1-859 | 860-1291 | ⁸⁵⁹EILNNI⁸⁶⁴ |
| | H9CNK9.1) | | | |
| BoNT/B8 | JQ964806.1 (aka | 1-859 | 860-1292 | ⁸⁵⁹EILNNI⁸⁶⁴ |
| | I6Z8G9.1) | | | |

Using structural analysis and sequence alignments, it was found that the β-strand following the 3₁₀ helix separating the LH_{N} and H_{C} domains is a conserved structure in all botulinum and tetanus neurotoxins and starts at the 8^{th} residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A covalently linked to a He domain from BoNT/B, wherein the C-terminal amino acid residue of the LH_{N} domain corresponds to the eighth amino acid residue N-terminally to the β-strand located at the beginning (N-term) of the H_{C} domain of BoNT/A, and wherein the N-terminal amino acid residue of the H_{C} domain corresponds to the seventh amino acid residue N-terminally to the β-strand located at the beginning (N-term) of the H_{C} domain of BoNT/B.

A BoNT/AB chimera may comprise an LH_{N} domain from BoNT/A covalently linked to a He domain from BoNT/B, wherein the C-terminal amino acid residue of the LH_{N} domain corresponds to the C-terminal amino acid residue of the α-helix located at the end (C-terminus) of the LH_{N} domain of BoNT/A, and wherein the N-terminal amino acid residue of the H_{C} domain corresponds to the amino acid residue immediately C-terminal to the C-terminal amino acid residue of the α-helix located at the end (C-terminus) of the LH_{N} domain of BoNT/B.

The rationale of the design process of the BoNT/AB chimera was to try to ensure that the secondary structure was not compromised and thereby minimise any changes to the tertiary structure and to the function of each domain. Without wishing to be bound by theory, it is hypothesized that by not disrupting the four central amino acid residues of the 3₁₀ helix in the BoNT/AB chimera ensures an optimal conformation for the chimeric neurotoxin, thereby allowing for the chimeric neurotoxin to exert its functions to their full capacity. In fact, surprisingly, retaining solely the first amino acid residue of the 3₁₀ helix of the BoNT/A and the second amino acid residue of the 3₁₀ helix onwards of BoNT/B not only allows the production of soluble and functional BoNT/AB chimera, but further leads to improved properties over other BoNT/AB chimeras, in particular an increased potency, an increased Safety Ratio and/or a longer duration of action (as well as increased Safety Ratio and/or duration of action when compared to unmodified BoNT/A—e.g. SEQ ID NO: 2).

The BoNT/A light-chain, BoNT/A translocation domain, and/or BoNT/B H_{C} domain may be a modified BoNT/A light-chain, BoNT/A translocation domain, and/or BoNT/B H_{C} domain or a derivative thereof, including but not limited to those described below. A modified BoNT/A light-chain, BoNT/A translocation domain, and/or BoNT/B H_{C} domain or derivative may contain one or more amino acids that has been modified as compared to the native (unmodified) form of the BoNT/A light-chain, BoNT/A translocation domain, and/or BoNT/B He domain, or may contain one or more inserted amino acids that are not present in the native (unmodified) form of the BoNT/A light-chain, BoNT/A translocation domain, and/or BoNT/B H_{C} domain. By way of example, a modified BoNT/A light-chain, BoNT/A translocation domain, and/or BoNT/B H_{C} domain may have modified amino acid sequences in one or more domains relative to the native (unmodified) BoNT/A light-chain, BoNT/A translocation domain, and/or BoNT/B H_{C} domain sequence. Such modifications may modify functional aspects thereof, for example biological activity or persistence. Thus, in one embodiment, the BoNT/A light-chain, BoNT/A translocation domain, and/or BoNT/B He domain is a modified BoNT/A light-chain, BoNT/A translocation domain, and/or BoNT/B He domain, or modified BoNT/A light-chain, BoNT/A translocation domain, and/or BoNT/B He domain derivative.

A modified BoNT/B H_{C} domain may have one or more modifications modifying binding to target nerve cells, for example providing higher or lower affinity binding when compared to the native (unmodified) BoNT/B H_{C} domain. Such modifications in the BoNT/B H_{C} domain may include modifying residues in the ganglioside binding site of the H_{C} domain or in the protein (e.g. synaptotagmin) binding site that alter binding to the ganglioside receptor and/or the protein receptor of the target nerve cell. Examples of such modified neurotoxins are described in WO 2006/027207 and WO 2006/114308, both of which are hereby incorporated by reference in their entirety.

A modified light-chain may have one or more modifications in the amino acid sequence thereof, for example modifications in the substrate binding or catalytic domain which may alter or modify the SNARE protein specificity of the modified light-chain, preferably with the proviso that said modifications do not catalytically inactivate said light-chain. Examples of such modified neurotoxins are described in WO 2010/120766 and US 2011/0318385, both of which are hereby incorporated by reference in their entirety.

The LH_{N} domain from BoNT/A may correspond to amino acid residues 1 to 872 of SEQ ID NO: 2, or a polypeptide sequence having at least 70% sequence identity thereto. The LH_{N} domain from BoNT/A may correspond to amino acid residues 1 to 872 of SEQ ID NO: 2, or a polypeptide sequence having at least 80%, 90% or 95% sequence identity thereto. Preferably, the LH_{N} domain from BoNT/A corresponds to amino acid residues 1 to 872 of SEQ ID NO: 2.

The H_{C} domain from BoNT/B may correspond to amino acid residues 860 to 1291 of SEQ ID NO: 8, or a polypeptide sequence having at least 70% sequence identity thereto. The He domain from BoNT/B may correspond to amino acid residues 860 to 1291 of SEQ ID NO: 8, or a polypeptide sequence having at least 80%, 90% or 95% sequence identity thereto. Preferably, the H_{C} domain from BoNT/B corresponds to amino acid residues 860 to 1291 of SEQ ID NO: 8.

Preferably, the BoNT/AB chimera comprises a BoNT/A1 LH_{N} domain and a BoNT/B1 He domain. More preferably, the LH_{N} domain corresponds to amino acid residues 1 to 872 of BoNT/A1 (SEQ ID NO: 2) and the H_{C} domain corresponds to amino acid residues 860 to 1291 of BoNT/B1 (SEQ ID NO: 8).

Most preferably, a BoNT/B H_{C} domain further comprises at least one amino acid residue substitution, insertion, indel or deletion in the H_{CC} subdomain which has the effect of increasing the binding affinity of BoNT/B neurotoxin for human Syt II as compared to the natural BoNT/B sequence. Suitable amino acid residue substitutions, insertions, indels or deletions in the BoNT/B H_{CC} subdomain have been disclosed in WO 2013/180799 and in WO 2016/154534 (both herein incorporated by reference).

A suitable amino acid residue substitution, insertion, indel or deletion in the BoNT/B H_{CC} subdomain may include a substitution mutation selected from the group consisting of: V1118M; Y1183M; E1191M; E11911; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

A suitable amino acid residue substitution, insertion, indel or deletion in the BoNT/B H_{CC} subdomain may further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

A suitable amino acid residue substitution, insertion, indel or deletion in the BoNT/B H_{CC} subdomain may also include a combination of three substitution mutations which are E1191M, S1199W and W1178Q.

Preferably, the amino acid residue substitution, insertion, indel or deletion in the BoNT/B H_{CC} subdomain includes a combination of two substitution mutations which are E1191M and S1199Y. Such modifications are present in modified BoNT/A (e.g. BoNT/AB chimeras) of SEQ ID NO: 5 and SEQ ID NO: 6, for example. E1191M may correspond to position 1204 of SEQ ID NO: 6 and S1199Y may correspond to position 1212. Thus, SEQ ID NO: 6 may comprise 1204M and 1212Y.

The modification may be a modification when compared to unmodified BoNT/B shown as SEQ ID NO: 8, wherein the amino acid residue numbering is determined by alignment with SEQ ID NO: 8. As the presence of a methionine residue at position 1 of SEQ ID NO: 8 (as well as the SEQ ID NOs corresponding to modified BoNT/A polypeptides described herein) is optional, the skilled person will take the presence/absence of the methionine residue into account when determining amino acid residue numbering. For example, where SEQ ID NO: 8 includes a methionine, the position numbering will be as defined above (e.g. E1191 will be E1191 of SEQ ID NO: 8). Alternatively, where the methionine is absent from SEQ ID NO: 8 the amino acid residue numbering should be modified by −1 (e.g. E1191 will be E1190 of SEQ ID NO: 8). Accordingly, an initial methionine amino acid residue of a polypeptide sequence of the modified BoNT/A may be optional or absent. Similar considerations apply when the methionine at position 1 of the other polypeptide sequences described herein is present/absent, and the skilled person will readily determine the correct amino acid residue numbering using techniques routine in the art.

A modified BoNT/A for use in the invention may comprise a polypeptide sequence having at least 70% sequence identity to a polypeptide sequence selected from SEQ ID NOs: 3-7. For example, a polypeptide sequence having at least 80%, 90%, 95% or 99.9% sequence identity to a polypeptide sequence selected from SEQ ID NOs: 3-7. Preferably, a modified BoNT/A for use in the invention may comprise (more preferably consist of) a polypeptide sequence selected from SEQ ID NOs: 3-7.

It is preferred that the modified BoNT/A comprises a polypeptide sequence having at least 70% sequence identity to SEQ ID NO: 6. For example, a polypeptide sequence having at least 80%, 90%, 95% or 99.9% sequence identity to SEQ ID NO: 6. Most preferably, a modified BoNT/A for use in the invention may comprise (more preferably consist of) SEQ ID NO: 6.

The term “deletion” as used herein refers to removal of one or more amino acid residues of a polypeptide without replacement of one or more amino acid residues at the site of deletion.

Thus, where one amino acid residue has been deleted from a polypeptide sequence having x number of amino acid residues (for example), the resultant polypeptide has x−1 amino acid residues.

The term “indel” as used herein refers to deletion of one or more amino acid residues of a polypeptide and insertion at the deletion site of a different number of amino acid residues (either greater or fewer amino acid residues) when compared to the number of amino acid residues deleted. Thus, for an indel where two amino acid residues have been deleted from a polypeptide sequence having x number of amino acid residues (for example), the resultant polypeptide has x−1 amino acid residues or x+≥1 amino acid residues. The insertion and deletion can be carried out in any order, sequentially or simultaneously.

The term “substitution” as used herein refers to replacement of one or more amino acid residues with the same number of amino acid residues at the same site. Thus, for a substitution of a polypeptide sequence having x number of amino acid residues (for example), the resultant polypeptide also has x amino acid residues. Preferably a substitution is a substitution at a single amino acid position.

The term “insertion” as used herein refers to addition of one or more amino acid residues of a polypeptide without deletion of one or more amino acid residues of the polypeptide at the site of insertion. Thus, where one amino acid residue has been inserted into a polypeptide sequence having x number of amino acid residues (for example), the resultant polypeptide has x+1 amino acid residues.

Methods for modifying proteins by substitution, insertion or deletion of amino acid residues are known in the art. By way of example, amino acid modifications may be introduced by modification of a DNA sequence encoding a BoNT/A (e.g. encoding unmodified BoNT/A). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised. Typically a modification may be carried out by either modifying a nucleic acid encoding a native clostridial neurotoxin (or part thereof) such that the modified BoNT/A (or part thereof) encoded by the nucleic acid comprises the modification(s). Alternatively, a nucleic acid that encodes a modified clostridial neurotoxin (or part thereof) comprising the modification(s) may be synthesized.

Where a polypeptide sequence of a modified BoNT/A described herein comprises a tag, e.g. for purification, such as a His-tag, said tag is optional. Preferably, said tag is removed prior to use of the modified BoNT/A according to the invention.

As discussed above, a modified BoNT/A described herein has increased tissue retention properties that also provide increased potency and/or duration of action and can allow for increased dosages without any additional negative effects. One way in which these advantageous properties may be defined is in terms of the Safety Ratio of the modified BoNT/A. In this regard, undesired effects of a clostridial toxin (caused by diffusion of the toxin away from the site of administration) can be assessed experimentally by measuring percentage bodyweight loss in a relevant animal model (e.g. a mouse, where loss of bodyweight is detected within seven days of administration). Conversely, desired on-target effects of a clostridial toxin can be assessed experimentally by Digital Abduction Score (DAS) assay, a measurement of muscle paralysis. The DAS assay may be performed by injection of 20 μl of clostridial neurotoxin, formulated in Gelatin Phosphate Buffer, into the mouse gastrocnemius/soleus complex, followed by assessment of Digital Abduction Score using the method of Aoki (Aoki K R, Toxicon 39: 1815-1820; 2001). In the DAS assay, mice are suspended briefly by the tail in order to elicit a characteristic startle response in which the mouse extends its hind limbs and abducts its hind digits. Following clostridial neurotoxin injection, the varying degrees of digit abduction are scored on a five-point scale (0=normal to 4=maximal reduction in digit abduction and leg extension).

The Safety Ratio of a neurotoxin (e.g. modified BoNT/A of the invention (or unmodified BoNT/A for comparison)) may then be expressed as the ratio between the amount of toxin required for a 10% drop in a bodyweight (measured at peak effect within the first seven days after dosing in a mouse) and the amount of neurotoxin required for a DAS score of 2. High Safety Ratio scores are therefore desired and indicate a neurotoxin that is able to effectively paralyse a target muscle with little undesired off-target effects. A modified BoNT/A of the present invention has a Safety Ratio that is higher than the Safety Ratio of an equivalent unmodified (native) BoNT/A.

A high Safety Ratio is particularly advantageous in therapy because it represents an increase in the therapeutic index. In other words, this means that reduced dosages can be used compared to alternative clostridial neurotoxin therapeutics and/or that increased dosages can be used without any additional (e.g. deleterious) effects. The possibility to use higher doses of neurotoxin without additional effects is particularly advantageous as higher doses usually lead to a longer duration of action of the neurotoxin.

The potency of a modified BoNT/A may be expressed as the minimal dose of neurotoxin which leads to a given DAS score when administered to a mouse gastrocnemius/soleus complex, for example a DAS score of 2 (ED₅₀ dose) or a DAS score of 4. The Potency of a modified BoNT/A may be also expressed as the EC₅₀ dose in a cellular assay measuring SNARE cleavage by the neurotoxin, for example the EC₅₀ dose in a cellular assay measuring SNAP25 cleavage by a modified BoNT/A.

The duration of action of a modified BoNT/A may be expressed as the time required for retrieving a DAS score of 0 after administration of a given dose of neurotoxin, for example the minimal dose of neurotoxin leading to a DAS score of 4, to a mouse gastrocnemius/soleus complex.

Thus, in one embodiment, a modified BoNT/A of the present invention has a Safety Ratio that is greater than 7 (for example, at least 8, 9, 10, 15, 20, 25, 30, 35, 40, 45 or 50), wherein Safety Ratio is calculated as: dose of toxin required for −10% bodyweight change (pg/mouse) divided by DAS ED₅₀ (pg/mouse) [ED₅₀=dose required to produce a DAS score of 2]. For example, a modified BoNT/A may have a Safety Ratio of at least 8, 9, 10, 15, 20, 25, 30, 35, 40, 45 or 50.

In one embodiment, a modified BoNT/A of the present invention has a Safety Ratio of at least 10. In one embodiment, a modified BoNT/A of the present invention has a Safety Ratio of at least 15. Preferably, the modified BoNT/A has a Safety Ratio of at least 10 (e.g. a Safety Ratio of 10), more preferably at least 12 or 13 (e.g. 14-15).

A modified BoNT/A for use in the invention may comprise a polypeptide sequence having at least 70% sequence identity to a polypeptide sequence selected from SEQ ID NOs: 3-7. For example, a polypeptide sequence having at least 80%, 90%, 95% or 99.9% sequence identity to a polypeptide sequence selected from SEQ ID NOs: 3-7. Preferably, a modified BoNT/A for use in the invention may comprise (more preferably consist of) a polypeptide sequence selected from SEQ ID NOs: 3-7. Of said modified BoNT/As, SEQ ID NO: 6 is preferred.

Thus, it is preferred that the modified BoNT/A comprises a polypeptide sequence having at least 70% sequence identity to SEQ ID NO: 6. More preferably, a polypeptide sequence having at least 80%, 90%, 95% or 99.9% sequence identity to SEQ ID NO: 6. Most preferably, a modified BoNT/A for use in the invention may comprise (more preferably consist of) SEQ ID NO: 6.

A di-chain modified BoNT/A of the invention may comprise an L-chain portion of a polypeptide sequence having at least 70%, 80%, 90%, 95%, 99.9%, or 100% sequence identity to any one of SEQ ID NOs: 3-7 constituting a first chain of the di-chain modified BoNT/A, and may comprise the H_{N} and H_{C} domains of a polypeptide sequence having at least 70%, 80%, 90%, 95%, 99.9%, or 100% sequence identity to any one of SEQ ID NOs: 3-7 together constituting a second chain of the di-chain modified BoNT/A, wherein the first and second chains are joined together by a di-sulphide bond.

Where cleavage occurs at more than one position (preferably at two positions) within the activation loop of a modified BoNT/A comprising a polypeptide sequence having at least 70%, 80%, 90%, 95%, 99.9%, or 100% sequence identity to any one of SEQ ID NOs: 3-7, a small fragment of the C-terminal L-chain portion of the sequence having at least 70%, 80%, 90%, 95%, 99.9%, or 100% sequence identity to any one of SEQ ID NOs: 3-7 may be absent from the di-chain modified BoNT/A. In view of this, the sequence of the di-chain modified BoNT/A (e.g. comprising a polypeptide sequence having at least 70%, 80%, 90%, 95%, 99.9%, or 100% sequence identity to any one of SEQ ID NOs: 3-7) may be slightly different to that of the corresponding single-chain modified BoNT/A comprising a polypeptide sequence having at least 70%, 80%, 90%, 95%, 99.9%, or 100% sequence identity to any one of SEQ ID NOs: 3-7. The small fragment may be 1-15 amino acids. In particular, in one embodiment, when Lys-C is used to covert a single-chain modified BoNT/A into a di-chain clostridial neurotoxin, the small fragment of the C-terminal L-chain portion of the sequence that is absent may be SEQ ID NO: 9 or 10.

Preferably, a di-chain modified BoNT/A of the invention may comprise an L-chain portion of a polypeptide sequence having at least 70%, 80%, 90%, 95%, 99.9%, or 100% sequence identity to SEQ ID NO: 6 constituting a first chain of the di-chain modified BoNT/A, and may comprise the H_{N} and H_{C} domains of a polypeptide sequence having at least 70%, 80%, 90%, 95%, 99.9%, or 100% sequence identity to SEQ ID NO: 6 together constituting a second chain of the di-chain modified BoNT/A, wherein the first and second chains are joined together by a di-sulphide bond.

Where cleavage occurs at more than one position (preferably at two positions) within the activation loop of a modified BoNT/A comprising a polypeptide sequence having at least 70%, 80%, 90%, 95%, 99.9%, or 100% sequence identity to SEQ ID NO: 6, a small fragment of the C-terminal L-chain portion of the sequence having at least 70%, 80%, 90%, 95%, 99.9%, or 100% sequence identity to SEQ ID NO: 6 may be absent from the di-chain modified BoNT/A. In view of this, the sequence of the di-chain modified BoNT/A (e.g. comprising a polypeptide sequence having at least 70%, 80%, 90%, 95%, 99.9%, or 100% sequence identity to SEQ ID NO: 6) may be slightly different to that of the corresponding single-chain modified BoNT/A comprising a polypeptide sequence having at least 70%, 80%, 90%, 95%, 99.9%, or 100% sequence identity to SEQ ID NO: 6. The small fragment may be 1-15 amino acids. In particular, in one embodiment, when Lys-C is used to covert a single-chain modified BoNT/A into a di-chain modified BoNT/A, the small fragment of the C-terminal L-chain portion of the sequence that is absent may be SEQ ID NO: 9 or 10.

In a particularly preferred embodiment, a di-chain modified BoNT/A comprises (or consists of) a light-chain comprising a polypeptide sequence having at least 70%, 80%, 90%, 95%, or 99.9% sequence identity to SEQ ID NO: 11 or 12 (preferably SEQ ID NO: 11) and a heavy-chain comprising a polypeptide sequence having at least 70%, 80%, 90%, 95%, or 99.9% sequence identity to SEQ ID NO: 13, wherein the light-chain and heavy-chain are joined together by a di-sulphide bond. More preferably, a di-chain modified BoNT/A comprises (or consists of) a light-chain comprising SEQ ID NO: 11 or 12 (preferably SEQ ID NO: 11) and a heavy-chain comprising SEQ ID NO: 13, wherein the light-chain and heavy-chain are joined together by a di-sulphide bond. Even more preferably, a di-chain modified BoNT/A comprises (or consists of) a light-chain having SEQ ID NO: 11 and a heavy-chain having SEQ ID NO: 13, wherein the light-chain and heavy-chain are joined together by a di-sulphide bond. The di-sulphide bond is preferably formed by and/or is between cysteine residue 429 of SEQ ID NO: 11 or 12 and cysteine residue 6 of SEQ ID NO: 13.

In a preferred embodiment, a modified BoNT/A of the invention does not comprise a therapeutic or diagnostic agent (e.g. a nucleic acid, protein, peptide or small molecule therapeutic or diagnostic agent) additional to the light-chain and heavy-chain. For example, in one embodiment, the modified BoNT/A may not comprise a covalently or non-covalently associated therapeutic or diagnostic agent. Thus, a modified BoNT/A of the invention preferably does not function as a delivery vehicle for a further therapeutic or diagnostic agent.

In embodiments where a modified BoNT/A described herein has a tag for purification (e.g. a His-tag) and/or a linker, said tag and/or linker are optional.

The modified BoNT/A is preferably in a non-complexed form (i.e. may be free from complexing proteins that are present in naturally occurring clostridial neurotoxin complex e.g. BoNT/A complex). Examples of such complexing proteins include a neurotoxin-associated proteins (NAP) and a nontoxic-nonhemagglutinin component (NTNH). However, it is preferred that the modified BoNT/A is a recombinant modified BoNT/A. The modified BoNT/A of the present invention can be produced using recombinant nucleic acid technologies. Thus, in one embodiment, a modified BoNT/A (as described herein) is a recombinant modified BoNT/A.

In one embodiment a nucleic acid (for example, DNA) comprising a nucleic acid sequence encoding a modified BoNT/A is provided. In one embodiment, the nucleic acid sequence is prepared as part of a DNA vector comprising a promoter and a terminator. The nucleic acid sequence may be selected from any of the nucleic acid sequences described herein.

In a preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |

In another preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |
| T5-lac operator | IPTG | 0.2 mM (0.05-2.0 mM) |

The nucleic acid molecules may be made using any suitable process known in the art. Thus, the nucleic acid molecules may be made using chemical synthesis techniques. Alternatively, the nucleic acid molecules of the invention may be made using molecular biology techniques.

The DNA construct of the present invention is preferably designed in silico, and then synthesised by conventional DNA synthesis techniques.

The above-mentioned nucleic acid sequence information is optionally modified for codon-biasing according to the ultimate host cell (e.g. E. coli) expression system that is to be employed.

The terms “nucleotide sequence” and “nucleic acid” are used synonymously herein. Preferably the nucleotide sequence is a DNA sequence.

A modified BoNT/A of the invention may be present as a single-chain or as a di-chain. However, it is preferred that the modified BoNT/A is present as a di-chain in which the L-chain is linked to the H-chain (or component thereof, e.g. the H_{N} domain) via a di-sulphide bond.

Production of a single-chain modified BoNT/A having a light-chain and a heavy-chain may be achieved using a method comprising expressing a nucleic acid encoding a modified BoNT/A in an expression host, lysing the host cell to provide a host cell homogenate containing the single-chain modified BoNT/A, and isolating the single-chain modified BoNT/A. The single-chain modified BoNT/A described herein may be proteolytically processed using a method comprising contacting a single-chain modified BoNT/A with a protease (e.g. Lys-C) that hydrolyses a peptide bond in the activation loop of the modified BoNT/A, thereby converting the single-chain modified BoNT/A into a corresponding di-chain modified BoNT/A (e.g. wherein the light-chain and heavy-chain are joined together by a disulphide bond). A di-chain modified BoNT/A is preferably obtainable by such a method.

Thus, a modified BoNT/A used in the invention is preferably a di-chain modified BoNT/A that has been produced from a single-chain BoNT/A, wherein the single-chain BoNT/A comprises or consists of a polypeptide sequence described herein. For example, it is preferred that the modified BoNT/A used in the invention is a di-chain modified BoNT/A that has been produced from a polypeptide comprising a polypeptide sequence having at least 70% (e.g. at least 80%, 90%, 95% or 99.9%) sequence identity to SEQ ID NO: 6. Most preferably, the modified BoNT/A used in the invention is a di-chain modified BoNT/A that has been produced from a polypeptide comprising (even more preferably consisting of) SEQ ID NO: 6. Accordingly, in some embodiments, the modified BoNT/A is a di-chain modified BoNT/A in which the light-chain (L-chain) is linked to the heavy-chain (H-chain) via a di-sulphide bond obtainable by a method comprising contacting a single-chain modified BoNT/A comprising SEQ ID NO: 6 with a protease that hydrolyses a peptide bond in the activation loop thereof, thereby converting the single-chain modified BoNT/A into the corresponding di-chain modified BoNT/A. In some embodiments, the modified BoNT/A is a di-chain modified BoNT/A in which the L-chain is linked to the H-chain via a di-sulphide bond obtainable by a method comprising contacting a single-chain modified BoNT/A consisting of SEQ ID NO: 6 with a protease that hydrolyses a peptide bond in the activation loop thereof, thereby converting the single-chain modified BoNT/A into the corresponding di-chain modified BoNT/A.

The protease used to cleave the activation loop is preferably Lys-C. Suitable proteases and method for cleaving activation loops to produce di-chain clostridial neurotoxins are taught in WO 2014/080206, WO2014/079495, and EP2677029A2, which are incorporated herein by reference. Lys-C may cleave an activation loop C-terminal to one or more of the lysine residues present therein. Where Lys-C cleaves the activation loop more than once, the skilled person will appreciate that a small peptide of the activation loop of a di-chain modified BoNT/A may be absent when compared to a SEQ ID NO shown herein.

The term “obtainable” as used herein also encompasses the term “obtained”. In one embodiment the term “obtainable” means obtained.

The term “one or more” as used herein may mean at least 2, 3, 4, 5, 6, 7, 8, 9, 10, 15, or 20. In one embodiment, wherein “one or more” precedes a list, “one or more” may mean all of the members of the list. Similarly, the term “at least one” as used herein may mean at least 2, 3, 4, 5, 6, 7, 8, 9, 10, 15, or 20. In one embodiment, wherein “at least one” precedes a list, “at least one” may mean all of the members of the list.

The term “disorder” as used herein also encompasses a “disease”. In one embodiment the disorder is a disease.

The modified BoNT/A of the invention may be formulated in any suitable manner for administration to a subject, for example as part of a pharmaceutical composition. Such a pharmaceutical composition comprising a modified BoNT/A of the invention and a pharmaceutically acceptable carrier, excipient, adjuvant, propellant and/or salt.

Fluid dosage forms are typically prepared utilising the modified BoNT/A and a pyrogen-free sterile vehicle. The modified BoNT/A, depending on the vehicle and concentration used, can be either dissolved or suspended in the vehicle. In preparing solutions the modified BoNT/A can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for an administration route described herein, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Also provided is a unit dosage form of modified BoNT/A for treating blepharospasm, typical hemifacial spasm and/or atypical hemifacial spasm, the unit dosage form comprising:
(a) at least 10 Units (preferably 10 Units to 333 Units) of modified BoNT/A, wherein 1 Unit is an amount of the modified BoNT/A that corresponds to the calculated median lethal dose (LD50) in mice; or
(b) at least 240 pg (preferably 240 pg to 8,000 pg) of modified BoNT/A; and
(c) optionally a pharmaceutically acceptable carrier, excipient, adjuvant, and/or salt,
   wherein the modified BoNT/A comprises a BoNT/A light-chain and translocation domain, and a BoNT/B receptor binding domain (HC domain).

The unit dose may be 1,500 to 5,000 pg of modified BoNT/A, preferably 2,000 to 4,500 pg of modified BoNT/A. Examples of suitable unit doses include about 2,500 pg (e.g. 2,000 pg±10%) of modified BoNT/A; and about 4,000 pg (e.g. 4,000 pg±10%) of modified BoNT/A.

The unit dose may be 62.4 to 208 U of modified BoNT/A, preferably 83.2 to 187.2 U of modified BoNT/A. Examples of suitable unit doses include about 104 U (e.g. 104 U±10%) of modified BoNT/A; and about 166.4 U (e.g. 166.4 U±10%) of modified BoNT/A.

A unit dosage form for treating blepharospasm, typical hemifacial spasm and/or atypical hemifacial spasm may comprise 10 Units to 333 Units of modified BoNT/A. An upper limit of said range may be 300, 250, 200, 150, or 100 Units of modified BoNT/A, preferably wherein the upper limit is 250 Units. A lower limit of say range may be 40, 45, 50, 60, 65, 70, 75, 80, 85, 90, or 100 Units, preferably 50 Units. Preferably, a unit dosage form comprises 42 Units to 300 Units of modified BoNT/A, for example 200 Units to 300 Units of modified BoNT/A.

A unit dosage form for treating blepharospasm, typical hemifacial spasm and/or atypical hemifacial spasm may comprise 240 pg to 8,000 pg of modified BoNT/A. An upper limit may be of said range may be 7,500, 6,500, 5,500, 4,500, 3,500, 2,500, 1,500 or 500 pg of modified BoNT/A, preferably the upper limit is 5,500 pg. A lower limit of said range may be 750, 850, 950, 1000, 1500, 2000, 2,500, 3,000, 3,500, 4,000, 4,500 or 5,000 pg of modified BoNT/A, preferably the lower limit is 1000 pg. Preferably, a unit dosage form comprises 2000 pg to 7,000 pg of modified BoNT/A, e.g. 4,000 pg to 6,000 pg.

Another aspect of the invention provides a kit comprising:
(a) a unit dosage form of modified BoNT/A described herein, wherein the modified BoNT/A comprises a BoNT/A light-chain and translocation domain, and a BoNT/B receptor binding domain (H_{C} domain); and
(b) instructions for use of the same in treating blepharospasm; and
(c) optionally a diluent.

Another aspect of the invention provides a kit comprising:
(a) the unit dosage form of modified BoNT/A described herein, wherein the modified BoNT/A comprises a BoNT/A light-chain and translocation domain, and a BoNT/B receptor binding domain (H_{C} domain); and
(b) instructions for use of the same in treating typical hemifacial spasm; and
(c) optionally a diluent.

Another aspect of the invention provides a kit comprising:
(a) the unit dosage form of modified BoNT/A described herein, wherein the modified BoNT/A comprises a BoNT/A light-chain and translocation domain, and a BoNT/B receptor binding domain (H_{C} domain); and
(b) instructions for use of the same in treating atypical hemifacial spasm; and
(c) optionally a diluent.

The modified BoNT/A of the unit dosage form may comprise a polypeptide sequence having at least 70% sequence identity to to any one of SEQ ID NOs: 3-7. For example, a polypeptide sequence having at least 80%, 90%, 95% or 99.9% sequence identity to any one of SEQ ID NOs: 3-7.

It is preferred that the modified BoNT/A of the unit dosage form comprises a polypeptide sequence having at least 70% sequence identity to SEQ ID NO: 6. For example, a polypeptide sequence having at least 80%, 90%, 95% or 99.9% sequence identity to SEQ ID NO: 6. Most preferably, a modified BoNT/may comprise (more preferably consist of) SEQ ID NO: 6.

Embodiments related to the various therapeutic uses of the invention can be applied to the methods of the invention and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes); preferably this method is used to align a sequence with a subject sequence herein (e.g. SEQ ID NO: 2) to define amino acid position numbering as described herein.

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 1 | 1 | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
   lysine
   histidine
Acidic: glutamic acid
   aspartic acid
Polar: glutamine
   asparagine
Hydrophobic: leucine
   isoleucine
   valine
Aromatic: phenylalanine
   tryptophan
   tyrosine
Small: glycine
   alanine
   serine
   threonine
   methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a modified botulinum neurotoxin A” includes a plurality of such candidate agents and reference to “the modified botulinum neurotoxin A” includes reference to one or more modified botulinum neurotoxin As and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows SDS-PAGE of purified recombinant BoNT/AB chimera 1, 2 and 3A (SEQ ID NO: 3, 4 and 5 respectively). Lanes are labelled “Marker” (molecular weight marker), “-DTT” (oxidised BoNT/AB chimera sample), and “+DTT” (reduced BoNT/AB chimera sample).

FIG. 2 shows cleavage of SNAP-25 in rat spinal cord neurones by recombinant BoNT/AB chimera 1, 2 and 3A (SEQ ID NO: 3, 4 and 5 respectively). Cultured rat primary spinal cord neurons (SCN) were exposed to various concentrations of recombinant BoNT/AB chimera 1, 2 or 3A for 24 hours, at 37° C. in a humidified atmosphere with 10% CO₂. Cells were then lysed with 1× NuPAGE buffer supplemented with DTT and Benzonase. The samples were transferred to microcentrifuge tubes, heated for 5 min at 90° C. on heat block and stored at −20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 3 shows mouse digit abduction scoring assay. Mice were injected into the gastrocnemius-soleus complex muscles of one hind limb, under short general anaesthesia; muscle weakening was measured on a 0-4 scale using the digit abduction score (DAS). DAS max values were determined for each dose and plotted against dose and the data were fitted to a 4-parameter logistic equation, ED50 and dose leading to DAS 4 (DAS 4 dose) values were determined.

FIG. 4 shows SDS-PAGE of purified recombinant BoNT/AB chimera 3B and 3C (SEQ ID NO: 6 and 7 respectively). Lanes are labelled “Marker” (molecular weight marker), “-DTT” (oxidised BoNT/AB chimera sample), and “+DTT” (reduced BoNT/AB chimera sample).

FIG. 5 shows cleavage of SNAP-25 by unmodified BoNT/A and BoNT/AB chimera 3B and 3C (SEQ ID NO: 2, 6 and 7 respectively) in human induced pluripotent stem cell derived peripheral neurons (PERI.4U—Axiogenesis, Germany). PERI.4U cells were exposed to various concentrations of recombinant BoNT/A, or BoNT/AB chimera 3B or 3C for 24 hours, at 37° C. in a humidified CO₂ atmosphere containing 5% CO₂. Cells were then lysed with 1× NuPAGE buffer supplemented with DTT and Benzonase. The samples were transferred to microcentrifuge tubes, heated for 5 min at 90° C. on heat block and stored at −20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 6 shows duration of muscle weakening over time in the mouse digit abduction scoring assay. Mice were injected into the gastrocnemius-soleus complex muscles of one hind limb, under short general anaesthesia; muscle weakening was measured on a 0-4 scale using the digit abduction score (DAS). Animals of the group injected with the lowest dose that induced during the first four days of injection a DAS of 4 were monitored until complete recovery of the muscle weakness to a DAS of 0 (no observed muscle weakness).

FIG. 7 shows, for reference, typical injection sites for the intramuscular administration of modified BoNT/A when treating glabellar lines, forehead lines and lateral canthal lines.

FIG. 8 shows particularly suitable injection sites for the intramuscular administration of modified BoNT/A when treating blepharospasm. Positions (1) and (3) point to the lateral pretarsal orbicularis oculi of the upper lid; position (2) points to the medial pretarsal orbicularis oculi of the upper lid; position (4) points the lateral pretarsal orbicularis oculi of the lower lid; position (5) points to the corrugator(s); position (6) points to the procerus; position (7) points to a frontalis.

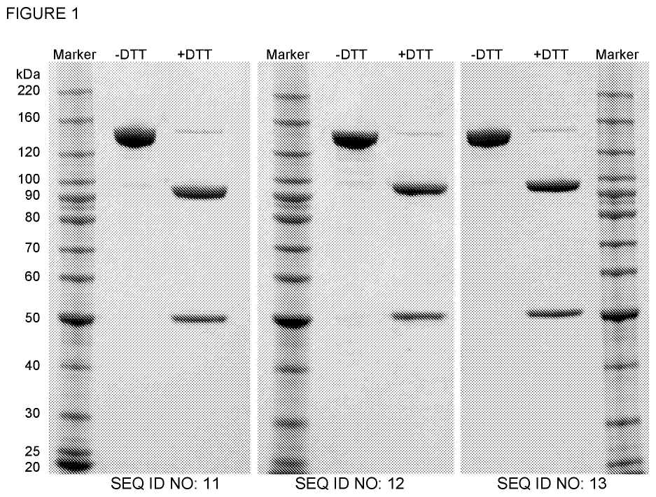

SEQUENCE LISTING

Where an initial Met amino acid residue or a corresponding initial codon is indicated
in any of the following SEQ ID NOs, said residue/codon is optional.
SEQ ID NO: 1 (Nucleotide Sequence of Unmodified BoNT/A)
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAATCATCTGATTGATCTGAGCCGTTATGCAAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAACGCCATTGTCTACAACTCCATGTACGAGAATTTCTCCACCAGCTTCTGGATTCGCATCCCG
AAATACTTCAACAGCATTAGCCTGAACAACGAGTATACTATCATCAACTGTATGGAGAACAACAGCGGTTGGAAG
GTGTCTCTGAACTATGGTGAGATCATTTGGACCTTGCAGGACACCCAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AACAGCAAGATTTACATCAATGGTCGCTTGATCGATCAGAAACCGATTAGCAACCTGGGTAATATCCACGCAAGC
AACAACATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACCAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
SEQ ID NO: 2 (Polypeptide Sequence of Unmodified BoNT/A)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 3 (Polypeptide Sequence of Modified BoNT/A “Chimera 1”)
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANENGQNTEINNMNFTKLKNFT
GLFEFYKLLCVRGIITSKTKSLDKGYNKALNDLCIKVNNWDLFFSPSEDNETNDLNKGEE
ITSDTNIEAAEENISLDLIQQYYLTENEDNEPENISIENLSSDIIGQLELMPNIEREPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDEVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDEDASLKDALLKYIYDNRGTLIGQVDRLKDK
VNNTLSTDIPFQLSKYVDNQRLLSTFTEYIKSEILNNIILNLRYKDNNLIDLSGYGAKVE
VYDGVELNDKNQFKLTSSANSKIRVTQNQNIIFNSVELDESVSEWIRIPKYKNDGIQNYI
HNEYTIINCMKNNSGWKISIRGNRIIWTLIDINGKTKSVFFEYNIREDISEYINRWEFVT
ITNNLNNAKIYINGKLESNTDIKDIREVIANGEIIFKLDGDIDRTQFIWMKYFSIENTEL
SQSNIEERYKIQSYSEYLKDEWGNPLMYNKEYYMENAGNKNSYIKLKKDSPVGEILTRSK
YNQNSKYINYRDLYIGEKFIIRRKSNSQSINDDIVRKEDYIYLDFFNLNQEWRVYTYKYF
KKEEMKLFLAPIYDSDEFYNTIQIKEYDEQPTYSCQLLFKKDEESTDEIGLIGIHRFYES
GIVFEEYKDYFCISKWYLKEVKRKPYNLKLGCNWQFIPKDEGWTEHHHHHHHHHH
SEQ ID NO: 4 (Polypeptide Sequence of Modified BoNT/A “Chimera 2”)
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSEGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKEDKLYKMLTEIYTEDNFVKFFKV
LNRKTYLNFDKAVFKINIVPKVNYTIYDGENLRNTNLAANENGQNTEINNMNFTKLKNFT
GLFEFYKLLCVRGIITSKTKSLDKGYNKALNDLCIKVNNWDLFFSPSEDNETNDLNKGEE
ITSDTNIEAAEENISLDLIQQYYLTENEDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINENIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIIELGGGGSELSEILNNIILNLRYKDNN
LIDLSGYGAKVEVYDGVELNDKNQFKLTSSANSKIRVTQNQNIIFNSVELDESVSFWIRI
PKYKNDGIQNYIHNEYTIINCMKNNSGWKISIRGNRIIWTLIDINGKTKSVFFEYNIRED
ISEYINRWFFVTITNNLNNAKIYINGKLESNTDIKDIREVIANGEIIFKLDGDIDRTQFI
WMKYFSIFNTELSQSNIEERYKIQSYSEYLKDFWGNPLMYNKEYYMFNAGNKNSYIKLKK
DSPVGEILTRSKYNQNSKYINYRDLYIGEKFIIRRKSNSQSINDDIVRKEDYIYLDFENL
NQEWRVYTYKYFKKEEMKLFLAPIYDSDEFYNTIQIKEYDEQPTYSCQLLFKKDEESTDE
IGLIGIHRFYESGIVFEEYKDYFCISKWYLKEVKRKPYNLKLGCNWQFIPKDEGWTEHHH
HHHHHHH
SEQ ID NO: 5 (Polypeptide Sequence of Modified BoNT/A “Chimera 3A”)
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSEGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGENLRNTNLAANENGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTENEDNEPENISIENLSSDIIGQLELMPNIEREPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDEDASLKDALLKYIYDNRGTLIGQVDRLKDK
VNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVELDESVSFWIRIPKYKNDGIQNYIH
NEYTIINCMKNNSGWKISIRGNRIIWTLIDINGKTKSVFFEYNIREDISEYINRWFFVTI
TNNLNNAKIYINGKLESNTDIKDIREVIANGEIIFKLDGDIDRTQFIWMKYESIENTELS
QSNIEERYKIQSYSEYLKDFWGNPLMYNKEYYMFNAGNKNSYIKLKKDSPVGEILTRSKY
NQNSKYINYRDLYIGEKFIIRRKSNSQSINDDIVRKEDYIYLDFENLNQEWRVYTYKYFK
KEEMKLFLAPIYDSDEFYNTIQIKEYDEQPTYSCQLLFKKDEESTDEIGLIGIHRFYESG
IVFEEYKDYFCISKWYLKEVKRKPYNLKLGCNWQFIPKDEGWTEHHHHHHHHHH
SEQ ID NO: 6 (Polypeptide Sequence of Modified BoNT/A “Chimera 3B”)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESGIVFEEYKDYFCISKW
YLKEVKRKPYNLKLGCNWQFIPKDEGWTE
SEQ ID NO: 7 (Polypeptide Sequence of Modified BoNT/A “Chimera 3C”)
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDESVSFWIRIPKYKNDGIQNYIH
NEYTIINCMKNNSGWKISIRGNRIIWTLIDINGKTKSVFFEYNIREDISEYINRWFFVTI
TNNLNNAKIYINGKLESNTDIKDIREVIANGEIIFKLDGDIDRTQFIWMKYFSIENTELS
QSNIEERYKIQSYSEYLKDFWGNPLMYNKEYYMFNAGNKNSYIKLKKDSPVGEILTRSKY
NQNSKYINYRDLYIGEKFIIRRKSNSQSINDDIVRKEDYIYLDFFNLNQEWRVYTYKYFK
KEEEKLFLAPISDSDEFYNTIQIKEYDEQPTYSCQLLFKKDEESTDEIGLIGIHRFYESG
IVFEEYKDYFCISKWYLKEVKRKPYNLKLGCNWQFIPKDEGWTE
SEQ ID NO: 8 (Polypeptide Sequence of BoNT/B)
MPVTINNFNYNDPIDNNNIIMMEPPFARGTGRYYKAFKITDRIWIIPERYTFGYKPEDEN
KSSGIFNRDVCEYYDPDYLNTNDKKNIFLQTMIKLENRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVENNVQENKGASIFNRRGYFSDPALILMHELIHVLHGLY
GIKVDDLPIVPNEKKFFMQSTDAIQAEELYTFGGQDPSIITPSTDKSIYDKVLQNERGIV
DRLNKVLVCISDPNININIYKNKFKDKYKFVEDSEGKYSIDVESFDKLYKSLMFGFTETN
IAENYKIKTRASYFSDSLPPVKIKNLLDNEIYTIEEGENISDKDMEKEYRGQNKAINKQA
YEEISKEHLAVYKIQMCKSVKAPGICIDVDNEDLFFIADKNSFSDDLSKNERIEYNTQSN
YIENDFPINELILDTDLISKIELPSENTESLTDFNVDVPVYEKQPAIKKIFTDENTIFQY
LYSQTFPLDIRDISLTSSFDDALLESNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDENDINSKLNEGINQAIDNINNFINGCSV
SYLMKKMIPLAVEKLLDEDNTLKKNLLNYIDENKLYLIGSAEYEKSKVNKYLKTIMPEDL
SIYINDTILIEMENKYNSEILNNIILNLRYKDNNLIDLSGYGAKVEVYDGVELNDKNQFK
LTSSANSKIRVTQNQNIIFNSVELDESVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSY
SEYLKDEWGNPLMYNKEYYMENAGNKNSYIKLKKDSPVGEILTRSKYNQNSKYINYRDLY
IGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
SEQ ID NO: 9-C-terminal L-chain Fragment
TKSLDKGYNK
SEQ ID NO: 10-C-terminal L-chain Fragment 2
SLDKGYNK
SEQ ID NO: 11-Di-Chain L-Chain 1
PFVNKQFNYKDPVNGVDIAYIKIPNAGQMQPVKAFKIHNKIWVIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEELNL
VIIGPSADIIQFECKSFGHEVLNLTRNGYGSTQYIRFSPDFTFGFEESLEVDTNPLLGAGKFATDPAVTLAHELI
HAGHRLYGIAINPNRVFKVNTNAYYEMSGLEVSFEELRTFGGHDAKFIDSLQENEFRLYYYNKFKDIASTLNKAK
SIVGTTASLQYMKNVFKEKYLLSEDTSGKFSVDKLKFDKLYKMLTEIYTEDNFVKFFKVLNRKTYLNEDKAVFKI
NIVPKVNYTIYDGFNLRNTNLAANFNGQNTEINNMNFTKLKNFTGLFEFYKLLCVRGIITSK
SEQ ID NO: 12-Di-Chain L-Chain 2
PFVNKQFNYKDPVNGVDIAYIKIPNAGQMQPVKAFKIHNKIWVIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEELNL
VIIGPSADIIQFECKSFGHEVLNLTRNGYGSTQYIRFSPDFTFGFEESLEVDTNPLLGAGKFATDPAVTLAHELI
HAGHRLYGIAINPNRVFKVNTNAYYEMSGLEVSFEELRTFGGHDAKFIDSLQENEFRLYYYNKFKDIASTLNKAK
SIVGTTASLQYMKNVFKEKYLLSEDTSGKFSVDKLKFDKLYKMLTEIYTEDNFVKFFKVLNRKTYLNEDKAVFKI
NIVPKVNYTIYDGFNLRNTNLAANFNGQNTEINNMNFTKLKNFTGLFEFYKLLCVRGIITSKTK
SEQ ID NO: 13-Di-Chain H-Chain
ALNDLCIKVNNWDLFFSPSEDNFTNDLNKGEEITSDTNIEAAEENISLDLIQQYYLTFNFDNEPENISIENLSSD
IIGQLELMPNIERFPNGKKYELDKYTMFHYLRAQEFEHGKSRIALTNSVNEALLNPSRVYTFFSSDYVKKVNKAT
EAAMFLGWVEQLVYDFTDETSEVSTTDKIADITIIIPYIGPALNIGNMLYKDDFVGALIFSGAVILLEFIPEIAI
PVLGTFALVSYIANKVLTVQTIDNALSKRNEKWDEVYKYIVTNWLAKVNTQIDLIRKKMKEALENQAEATKAIIN
YQYNQYTEEEKNNINFNIDDLSSKLNESINKAMININKFLNQCSVSYLMNSMIPYGVKRLEDFDASLKDALLKYI
YDNRGTLIGQVDRLKDKVNNTLSTDIPFQLSKYVDNQRLLSTFTEYIKNILNNIILNLRYKDNNLIDLSGYGAKV
EVYDGVELNDKNQFKLTSSANSKIRVTQNQNIIFNSVFLDFSVSFWIRIPKYKNDGIQNYIHNEYTIINCMKNNS
GWKISIRGNRIIWTLIDINGKTKSVFFEYNIREDISEYINRWFFVTITNNLNNAKIYINGKLESNTDIKDIREVI
ANGEIIFKLDGDIDRTQFIWMKYFSIFNTELSQSNIEERYKIQSYSEYLKDFWGNPLMYNKEYYMFNAGNKNSYI
KLKKDSPVGEILTRSKYNQNSKYINYRDLYIGEKFIIRRKSNSQSINDDIVRKEDYIYLDFFNLNQEWRVYTYKY
FKKEEMKLFLAPIYDSDEFYNTIQIKEYDEQPTYSCQLLFKKDEESTDEIGLIGIHRFYESGIVFEEYKDYFCIS
KWYLKEVKRKPYNLKLGCNWQFIPKDEGWTE

### EXAMPLES

### Example 1

Cloning, Expression and Purification of Modified BoNT/A (BoNT/AB Chimeras) BoNT/AB chimeric constructs 1, 2, 3A, 3B, and 3C (SEQ ID NO: 3-7, respectively) were constructed from DNA encoding the parent serotype molecule and appropriate oligonucleotides using standard molecular biology techniques. These were then cloned into the pJ401 expression vector with or without a C-terminal His₁₀-tag and transformed into BLR (DE3) E. coli cells for over-expression. These cells were grown at 37° C. and 225 RPM shaking in 2 L baffled conical flasks containing 1 L modified Terrific Broth (mTB) supplemented with the appropriate antibiotic. Once the A₆₀₀ reached >0.5, the incubator temperature was decreased to 16° C., and then induced with 1 mM IPTG an hour later for 20 h at 225 RPM shaking, to express the recombinant BoNT/AB construct.

Harvested cells were lysed by ultrasonication and clarified by centrifugation at 4500 RPM for 1 h at 4° C. The recombinant BoNT/AB chimeric molecules were then extracted in ammonium sulphate and purified by standard fast protein liquid chromatography (FPLC) techniques. This involved using a hydrophobic interaction resin for capture and an anion-exchange resin for the intermediate purification step. The partially purified molecules were then proteolytically cleaved with endoproteinase Lys-C to yield the active di-chain. This was further purified with a second hydrophobic interaction resin to obtain the final BoNT/AB chimera.

For BoNT/AB chimeric molecules with a decahistadine tag (H₁₀) (chimera 1, 2, 3A), the capture step employed the use of an immobilised nickel resin instead of the hydrophobic interaction resin.

The sequence of each chimera is presented in Table 1.

TABLE 1
chimeric BoNT/AB constructs

| | SEQ | |
|---|---|---|
| Molecule | ID NO | Sequence |
| Chimera 1 | 3 | A1: 1-871 + B1: 858-1291 (E1191M/S1199Y) + |
| | | His₁₀-tag |
| Chimera 2 | 4 | A1: 1-874 + ELGGGGSEL + B1: 858-1291 |
| | | (E1191M/S1199Y) + His₁₀-tag |
| Chimera 3A | 5 | A1: 1-872 + B1: 860-1291 (E1191M/S1199Y) + |
| | | His₁₀-tag |
| Chimera 3B | 6 | A1: 1-872 + B1: 860-1291 (E1191M/S1199Y) |
| Chimera 3C | 7 | A1: 1-872 + B1: 860-1291 |

### Example 2

Comparison of BoNT/AB chimera 1, 2 and 3A

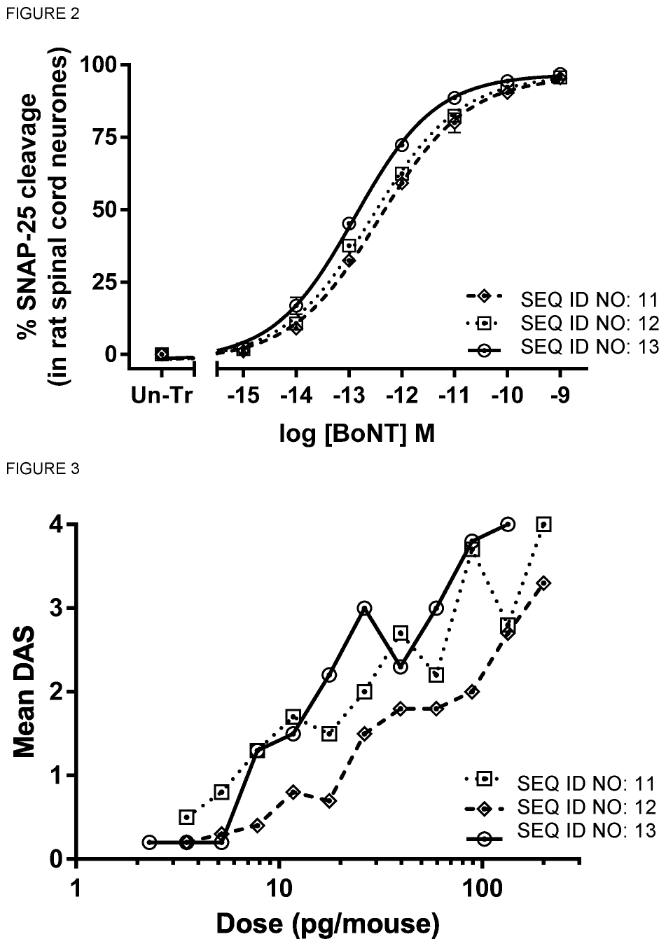

BoNT/AB chimera 1, 2 and 3A which have a C-terminal His₁₀ tag and E1191M/S1199Y double mutation were purified as described in Example 1 (FIG. 2) and tested for functional activity.

### Rat Spinal Cord Neurons SNAP-25 Cleavage Assay

Primary cultures of rat spinal cord neurons (SCN) were prepared and grown, for 3 weeks, in 96 well tissue culture plates (as described in: Masuyer et al., 2011, J. Struct. Biol. Structure and activity of a functional derivative of Clostridium botulinum neurotoxin B; and in: Chaddock et al., 2002, Protein Expr. Purif. Expression and purification of catalytically active, non-toxic endopeptidase derivatives of Clostridium botulinum toxin type A). Serial dilutions of BoNT/AB were prepared in SCN feeding medium. The growth medium from the wells to be treated was collected and filtered (0.2 m filter). 125 μL of the filtered medium was added back to each test well. 125 μL of diluted toxin was then added to the plate (triplicate wells). The treated cells were incubated at 37° C., 10% CO₂, for 24±1 h).

### Analysis of BoNT Activity Using the SNAP-25 Cleavage Assay

Following treatment, BoNT was removed and cells were washed once in PBS (Gibco, UK). Cells were lysed in 1× NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

Table 2 below provides the pEC₅₀ values determined for Chimera 1, 2 and 3A in the rat SCN SNAP-25 cleavage assay. These results show that the three BoNT/AB chimeras retained the ability to enter rat spinal cord neurons and cleave their target substrate. However, chimera 3A was more potent than chimera 1 and 2 in this assay (see also FIG. 2).

TABLE 2
pEC₅₀ values.

| | pEC₅₀ ± SEM |
|---|---|
| Chimera 1 | 12.42 ± 0.04 |
| Chimera 2 | 12.57 ± 0.01 |
| Chimera 3A | 12.89 ± 0.04 |

### Digit Abduction Scoring (DAS) Assay

The method to measure the activity of BoNT/AB chimera 1, 2 and 3A in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10).

On the day of injection, mice were anaesthetized in an induction chamber receiving isoflurane 3% in oxygen. Each mouse received an intramuscular injection of BoNT/AB chimera or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED₅₀ was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days.

FIG. 3 shows the fitted curves for chimera 1, 2 and 3A (SEQ ID NO: 3, 4 and 5 respectively). The chimera 3A curve is shifted to the left, meaning lower doses of chimera 3A achieved a similar DAS response compared to chimera 1 and 2, therefore showing that chimera 3A is more potent than the others in the mouse DAS assay; see also the table below (Table 3) that provides the values for the calculated ED₅₀ and the dose leading to DAS 4 (highest score) for each chimera.

Table 3 below provides the ED₅₀ and DAS 4 doses determined for unmodified recombinant BoNT/A1 (rBoNT/A1—SEQ ID NO: 2) and chimeras 1, 2 and 3A in the mouse DAS assay. These results show that of the three chimeras, chimera 3A has the highest in vivo potency in inducing muscle weakening. Studies shown in FIG. 3 and Table 3 were performed in mice obtained from Charles River laboratories.

TABLE 3
ED₅₀ values.

| | ED₅₀ | DAS 4 dose |
|---|---|---|
| | (pg/mouse) | (pg/mouse) |
| rBoNT/A1 | 1 | 5 |
| Chimera 1 | 23 | 200 |
| Chimera 2 | 89 | >300 |
| Chimera 3A | 18 | 133 |

### Example 3

### Comparison of BoNT/AB Chimera 3B, 3C and Unmodified BoNT/A1

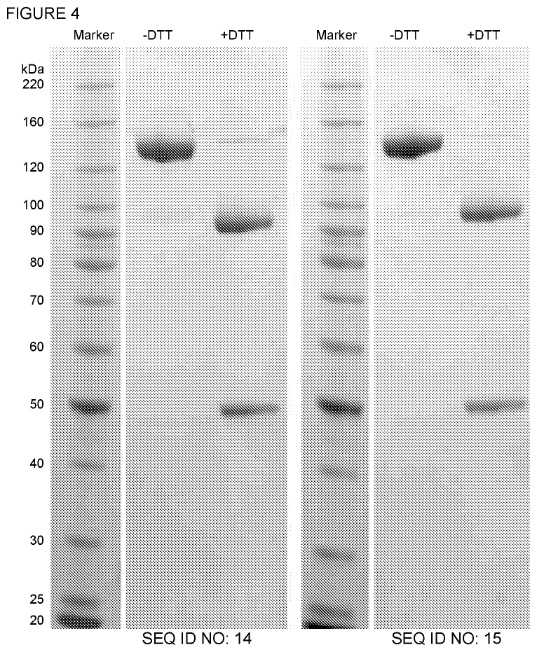

Untagged BoNT/AB chimera 3B and 3C, respectively with and without the presence of the E1191M/S1199Y double mutation (SEQ ID NO: 6 and 7) were purified as described in Example 3 (FIG. 4), and tested for functional activity using unmodified BoNT/A (SEQ ID NO: 2) as a reference.

### Human Pluripotent Stem Cells SNAP-25 Cleavage Assay

Cryopreserved PERI.4U-cells were purchased from Axiogenesis (Cologne, Germany). Thawing and plating of the cells were performed as recommended by the manufacturer. Briefly, cryovials containing the cells were thawed in a water bath at 37° C. for 2 minutes. After gentle resuspension the cells were transferred to a 50 mL tube. The cryovial was washed with 1 mL of Peri.4U® thawing medium supplied by the manufacturer and the medium was transferred drop-wise to the cell suspension to the 50 mL tube, prior to adding a further 2 mL of Peri.4U® thawing medium drop-wise to the 50 mL tube. Cells were then counted using a hemocytometer. After this, a further 6 mL of Peri.4U® thawing medium was added to the cell suspension. A cell pellet was obtained by centrifugation at 260×g (e.g. 1,100 RPM) for 6 minutes at room temperature. Cells were then resuspended in complete Peri.4U® culture medium supplied by the manufacturer. Cells were plated at a density of 50,000 to 150,000 cells per cm² on cell culture plates coated with poly-L-ornithine and laminin. Cells were cultured at 37° C. in a humidified CO₂ atmosphere, and medium was changed completely every 2-3 days during culture.

For toxin treatment, serial dilutions of BoNTs were prepared in Peri.4U® culture medium. The medium from the wells to be treated was collected and filtered (0.2 m filter). 125 μL of the filtered medium was added back to each test well. 125 μL of diluted toxin was then added to the plate (triplicate wells). The treated cells were incubated at 37° C., 10% CO₂, for 48±1 h).

### Analysis of BoNT Activity Using the SNAP-25 Cleavage Assay

Following treatment, BoNT was removed and cells were washed once in PBS (Gibco, UK). Cells were lysed in 1× NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

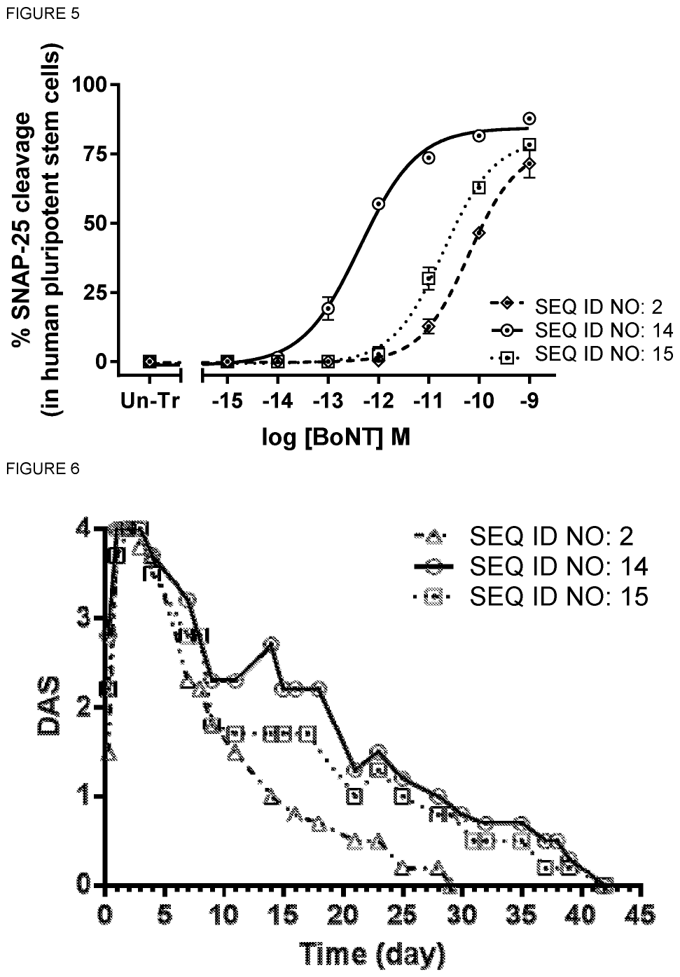

FIG. 5 shows that chimera 3B and 3C displayed greater potency than rBoNT/A1 in cleaving SNAP-25 in induced human pluripotent stem cells but the former significantly more so. This can be explained by the double mutation which increases the affinity of chimera 3B for the human synaptotagmin II protein receptor present in these cells (FIG. 5, Table 4).

TABLE 4
pEC₅₀ values.

| | pEC₅₀ ± SEM |
|---|---|
| rBoNT/A1 | 10.21 ± 0.05 |
| Chimera 3B | 12.38 ± 0.06 |
| Chimera 3C | 10.72 ± 0.08 |

### Digit Abduction Scoring (DAS) Assay—Safety Ratio

The method to measure the activity of BoNTs in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10).

On the day of injection, mice were anaesthetized in an induction chamber receiving isoflurane 3% in oxygen. Each mouse received an intramuscular injection of BoNT or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED₅₀ was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days for all doses. Animals of the groups injected with vehicle and the lowest dose that induced during the first four days of injection a DAS of 4 were thereafter monitored until complete recovery of the muscle weakness to a DAS of 0 (no observed muscle weakness).

For calculation of the safety ratio all animals were weighed the day before toxin injection (D0) and thereafter once daily throughout the duration of the study. The average body weight, its standard deviation, and the standard error mean were calculated daily for each dose-group. To obtain the safety ratio for a BoNT (−10% ΔBW/ED₅₀), the dose at which at any time during the study the average weight of a dose-group was lower than 10% of the average weight at D0 of that same dose-group was divided by the ED₅₀ for the BoNT studied. The lethal dose was defined as the dose at which one or more of the animals within that dose-group died.

FIG. 6 shows the duration of muscle weakening over time in the mouse digit abduction scoring assay for unmodified BoNT/A, chimera 3B and chimera 3C (SEQ ID NO: 2, 6 and 7 converted into a di-chain form), showing that the chimera has longer duration of action.

Table 5 below provides the ED₅₀ and DAS 4 doses determined for rBoNT/A1 and chimeras 3B and 3C in the mouse DAS assay. The table also provide the total duration of action for the DAS 4 dose until complete recovery of the muscle weakness to a DAS of 0 (no observed muscle weakness). In addition, the table shows the mouse lethal dose and the safety ratio (−10% ΔBW/ED₅₀), as defined in the text above. In comparison to rBoNT/A1, chimeras 3B and 3C have longer duration of action, a better safety ratio, and a higher lethal dose. Studies shown in FIG. 6 and Table 5 were performed in mice obtained from Janvier laboratories.

TABLE 5
DAS and Safety Ratios of the BoNT/AB chimeras.

| | ED₅₀ | | | | |
|---|---|---|---|---|---|
| | (DAS 2) | DAS 4 | Total duration | Mouse | Safety |
| | Dose | dose | of action (day) | lethal | ratio |
| | (pg/ | (pg/ | with lowest | dose | (−10%ΔBW/ |
| | mouse) | mouse) | DAS 4 dose | (pg) | ED₅₀) |
| rBoNT/A1 | 0.9 | 2.3 | 29 | 18 | 4.5 |
| Chimera | 8.0 | 89 | 42 | 200 | 14.1 |
| 3B | | | | | |
| Chimera | 5.0 | 26 | 42 | 8.9 | 7.4 |
| 3C | | | | | |

### Example 4

Pre-Clinical Testing of Modified BoNT/a (BoNT/AB Chimera [SEQ ID NO: 6 Converted into a Di-Chain Form])

BoNT/AB chimera SEQ ID NO: 6 converted into a di-chain form was tested in a mouse LD₅₀ assay yielding a result of 1.202 ng/kg. 1 Unit of SEQ ID NO: 6 therefore corresponds to 24.04 pg in this assay.

Additionally, said BoNT/AB chimera was tested in a rat DAS assay to determine the duration of action (as per Example 6) when compared to Dysport®. Results are presented in Table 6 below:

TABLE 6
Duration of action.

| | Dysport ® | BoNT/AB |
|---|---|---|
| | 3 U/rat | 300 pg/rat |
| | 15 U/kg | 1.5 ng/kg |
| Duration of Action (median days) | 21.9 | 47.7 |

In conclusion, the duration of action of BoNT/AB was much higher than Dysport®.

### Example 5

Determination of a Unit Dose of Modified BoNT/a (SEQ ID NO: 6 Converted into a Di-Chain Form) for Treating a Disorder Affecting an Eyelid Muscle of a Subject

In view of pre-clinical pharmacology data, a suitable unit dose (UD) for administration of modified BoNT/A in humans has been determined.

A DAS ED₅₀ of 13 pg/kg was calculated for SEQ ID NO: 6. ED₅₀ is considered as a minimal pharmacologically active dose, which is approximately 300-fold lower than the no observed adverse effect level (NOAEL) of 4 ng/kg in the same animal species. An ED₅₀ of 13 pg/kg of SEQ ID NO: 6 in rats corresponds to a 0.8 ng dose for a human of 60 kg body weight.

Thus, a dose of 1,000 pg was considered preferable. However, as above, given that 10 U Dysport® administered per site during treatment of a disorder affecting an eyelid muscle of a subject (e.g. blepharospasm or hemifacial spasm) is therapeutically effective, it was considered that a corresponding 10 U dose of modified BoNT/A (SEQ ID NO: 6) would also be an efficacious minimum unit dose (e.g. administered similarly to Dysport®). Using the intraperitoneal mouse LD₅₀ data above, 240.4 pg (rounded to 240 pg) of modified BoNT/A equates to approximately 10 U Dysport®.

The NOAEL is 4 ng/kg for both nonclinical safety species (rat and monkey), which when converted into human dose for 60 kg body weight, is 240,000 pg.

Out of an abundance of caution, an upper limit for treatment was selected at 24,000 pg (˜998 U), which is 10-times lower than the NOAEL.

Thus, a suitable treatment for a disorder affecting an eyelid muscle of a subject uses at least 240 pg (10 U) of modified BoNT/A up to a total dose during treatment of 24,000 pg (˜998 U). The upper limit of the unit dose may be determined based on the number of muscles and/or sites to which the modified BoNT/A is administered. For example, where the modified BoNT/A is administered to three muscles and/or sites (e.g. the lateral upper orbicularis oculi muscle, medial upper orbicularis oculi muscle, and lateral lower orbicularis oculi muscle in the treatment of unilateral blepharospasm) a suitable unit dose would be 240 pg to 8,000 pg (10 U to ˜332.7 U) of modified BoNT/A. If administered to six muscles/sites (e.g. 2× lateral upper orbicularis oculi muscle, 2× medial upper orbicularis oculi muscle, and 2× lateral lower orbicularis oculi muscle in the treatment of bilateral blepharospasm) a suitable unit dose would be 240 pg to 4,000 pg (10 U to ˜166.3 U) of modified BoNT/A. This ensures that the total dose is not exceeded.

In view of the improved safety profile when compared to Dysport® as determined by the pre-clinical data of Example 4, total dosages (in units) administered in treating a disorder affecting an eyelid muscle of a subject are expected to be just over 4× greater than that for Dysport®. The maximum total dose of Dysport® for treatment of blepharospasm and, separately, hemifacial spasm is 240 Units (120 Units per eye).

Advantageously, more modified BoNT/A (SEQ ID NO: 6) can be injected and/or can be injected at a greater number of muscles and/or sites in the treatment of disorders affecting an eyelid muscle of a subject before reaching the maximum dose. This is a significant and advantageous finding leading to improved treatment of such conditions while providing clinicians with a greater range of treatment options.

### Example 6

### Dosage Regimen for Treating a Disorder Affecting an Eyelid Muscle of a Subject Using a Modified BoNT/a (SEQ ID NO: 6)

Modified BoNT/A is provided as a lyophilised powder in a vial containing 36 ng of modified BoNT/A per vial. The lyophilised powder is reconstituted.

The unit dose (UD) is 240-8,000 pg (˜10-332.7 Units [measured by mouse LD₅₀]).

The disorder is treated by injection according to the following dosage regimen (Table 7):

TABLE 7
Dosage regimen.

| Muscle/Site | Dosage (Unit Dose) |
|---|---|
| Lateral upper orbicularis oculi | 1 × UD |
| Medial upper orbicularis oculi muscle | 1 × UD |
| Lateral lower orbicularis oculi | 1 × UD |

A maximum total dosage administered is 24,000 pg (˜998 U). This is just over 4× greater than the maximum total dosage of Dysport® that can be administered during treatment of blepharospasm or hemifacial spasm without approaching toxic limits (a concern with conventional treatment regimens). Thus, the clinician is able to tailor treatment to the patient with the knowledge that 24,000 pg (˜998 U) can be administered without any concern of toxicity, thereby allowing the treatment of additional muscles of the subject and/or ensuring each muscle and/or site thereof receives a pharmaceutically effective dose.

### Example 7

Treatment of a Patient with Blepharospasm

Loretta, aged 52, is diagnosed by her GP with bilateral blepharospasm. Modified BoNT/A (SEQ ID NO: 6) is administered to each of the following of Loretta's muscles/sites thereof:
1× unit dose (UD) of 2,000 pg to the lateral upper orbicularis oculi muscle of the left eye;
1× UD of 2,000 pg to the medial upper orbicularis oculi muscle of the left eye;
1× UD of 2,000 pg the lateral lower orbicularis oculi muscle of the left eye;
1× UD of 2,000 pg to the lateral upper orbicularis oculi muscle of the right eye;
1× UD of 2,000 pg to the medial upper orbicularis oculi muscle of the right eye; and
1× UD of 2,000 pg the lateral lower orbicularis oculi muscle of the right eye.

The total amount of modified BoNT/A is less than the 24,000 pg. The blepharospasm is alleviated and, owing to the long duration of the modified BoNT/A, Loretta does not require further treatment for 9 months. Thus, Loretta receives less frequent injections (e.g. per year) when compared to an equivalent subject administered an unmodified BoNT/A. Additionally, Loretta dose not exhibit any side-effects owing to the improved safety profile of the modified BoNT/A.

### Example 8

Treatment of a Patient with Blepharospasm

Eleanor, aged 63, is diagnosed by her GP with unilateral blepharospasm affecting the eyelid muscles proximal to her left eye. Modified BoNT/A (SEQ ID NO: 6) is administered to each of the following of Eleanor's muscles/sites thereof:
1× unit dose (UD) of 250 pg to the lateral upper orbicularis oculi muscle of the left eye;
1× UD of 250 pg to the medial upper orbicularis oculi muscle of the left eye;
1× UD of 250 pg the lateral lower orbicularis oculi muscle of the left eye; and
2× UD of 500 pg to the frontalis muscle of the left eye (at 2× sites).

The total amount of modified BoNT/A is less than the 2,000 pg. The blepharospasm is alleviated and, owing to the long duration of the modified BoNT/A, Eleanor does not require further treatment for greater than 9 months. Thus, Eleanor receives less frequent injections (e.g. per year) when compared to an equivalent subject administered an unmodified BoNT/A.

Additionally, Eleanor dose not exhibit any side-effects owing to the improved safety profile of the modified BoNT/A.

### Example 9

Treatment of a Patient with Hemifacial Spasm

Derek, aged 49, is diagnosed with hemifacial spasm affecting the left side of his face. Modified BoNT/A (SEQ ID NO: 6) is administered to each of the following of Derek's muscles/sites thereof:
1× unit dose (UD) of 3,000 pg to the lateral upper orbicularis oculi muscle of the left eye;
1× UD of 3,000 pg to the medial upper orbicularis oculi muscle of the left eye;
1× UD of 3,000 pg to the lateral lower orbicularis oculi muscle of the left eye;
1× UD of 3,000 pg to the corrugator muscle on the left side of the face;
1× UD of 3,000 pg to the frontalis muscle on the left side of the face;
1× UD of 3,000 pg to the zygomaticus major muscle on the left side of the face;
1× UD of 3,000 pg to the buccinator muscle on the left side of the face; and
1× UD of 3,000 pg to the masseter muscle on the left side of the face.

The total amount of modified BoNT/A is up to 24,000 pg. The hemifacial spasm is alleviated and, owing to the long duration of the modified BoNT/A, Derek does not require further treatment for greater than 9 months. Thus, Derek receives less frequent injections (e.g. per year) when compared to an equivalent subject administered an unmodified BoNT/A. Additionally, Derek dose not exhibit any side-effects owing to the improved safety profile of the modified BoNT/A.

### Example 10

Treatment of a Patient with Hemifacial Spasm

Kayleigh, aged 41, is diagnosed by her GP with hemifacial spasm affecting the eyelid muscles proximal to her right eye. Modified BoNT/A (SEQ ID NO: 6) is administered to each of the following of Kayleigh's muscles/sites thereof:
1× unit dose (UD) of 650 pg to the lateral upper orbicularis oculi muscle of the right eye;
1× UD of 650 pg to the medial upper orbicularis oculi muscle of the right eye; and
1× UD of 650 pg the lateral lower orbicularis oculi muscle of the right eye.

The total amount of modified BoNT/A is less than the upper limit of 2,000 pg. The hemifacial spasm is alleviated and, owing to the long duration of the modified BoNT/A, Kayleigh does not require further treatment for 9 months. Thus, Kayleigh receives less frequent injections (e.g. per year) when compared to an equivalent subject administered an unmodified BoNT/A. Additionally, Kayleigh dose not exhibit any side-effects owing to the improved safety profile of the modified BoNT/A.

### Example 11

### Safety & Efficacy of Modified BoNT/A (SEQ ID NO: 6) in Humans

An integrated phase I/II multicentre, double-blinded, randomised, Dysport and placebo controlled, dose-escalation and dose-finding study to evaluate the safety and efficacy of a modified BoNT/A (SEQ ID NO: 6) in the treatment of moderate to severe upper facial lines was carried out for adults. Said upper facial lines include glabellar lines, forehead lines and lateral canthal lines.

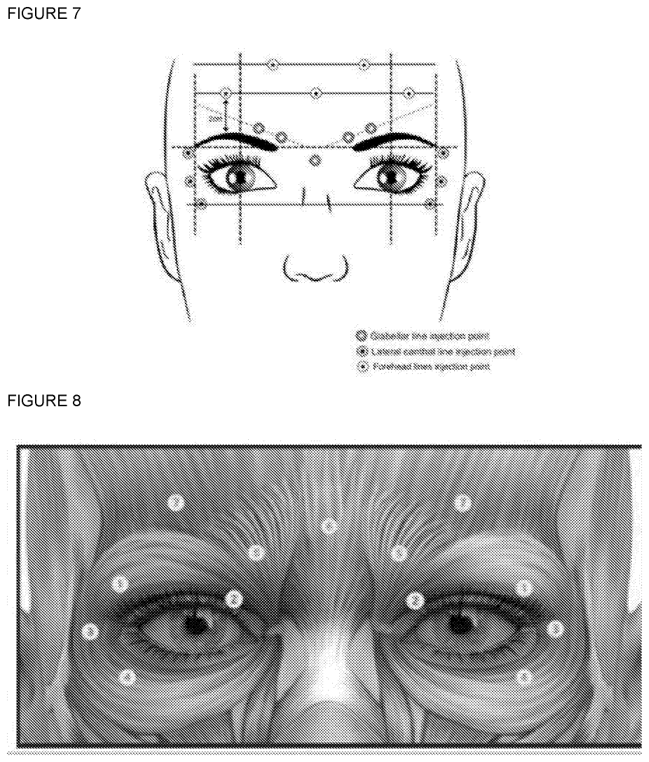

The study included a human sequential dose escalation in cohorts of unique patients with adult upper facial lines. Said upper facial lines include glabellar lines, forehead lines and lateral canthal lines. Each muscle was injected with a single unit dose of modified BoNT/A, Dysport, or placebo. 6 cohorts were administered different (increasing) amounts of modified BoNT/A (SEQ ID NO: 6). The total dose range was 0.1 to 6.0 ng. The modified BoNT/A was injected in up to 16 sites across the upper facial area (five sites in the glabellar lines, five sites in the forehead lines and three sites on each side in lateral canthal lines—see FIG. 7).

Results showed that all unit doses of modified BoNT/A tested and the total dose, (i.e. up to 6,000 pg), were effective, safely tolerated, and no adverse effects were observed, despite the exceptionally high dosage per muscle. This shows that the modified BoNT/A does not diffuse away from the injection site and highlights the exceptional safety profile of modified BoNT/A (SEQ ID NO: 6). Based on these findings, it is considered credible that much higher unit doses can be administered per muscle without resultant adverse effects. Furthermore, given the lack of systemic diffusion of the toxin, it is credible that up to 13-14× the higher unit doses can be administered without safety concerns.

Thus, unit doses of up to 5,000 pg+/−10% (each to be administered up to 16× across the upper facial area (e.g. five sites in the glabellar lines, five sites in the forehead lines and three sites on each side in lateral canthal lines) have been selected for treatment of upper facial lines. The total doses administered during a treatment session will, therefore, be up to 80,000 pg (+/−10%), respectively.

The muscles to which the modified BoNT/A is injected (see FIG. 7) widely overlaps with those injected in the facial area of a patient with a facial dystonia such as blepharospasm or hemifacial spasm. Thus, the present study (in respect of upper facial lines) also informs suitable and safe dosage strategies for treating a facial dystonia. In the case of facial dystonia, it is envisaged that up to 15 sites would be injected.

### Example 12

### Dosage Regimen for Treating Blepharospasm Using a Modified BoNT/A (SEQ ID NO: 6)

Modified BoNT/A is provided as a lyophilised powder in a vial containing 36 ng of modified BoNT/A per vial. The lyophilised powder is reconstituted.

The unit dose (UD) is 240-8,000 pg (˜10-332.7 Units [measured by mouse LD₅₀]).

The disorder is treated by injection according to the following dosage regimen (Table 8):

TABLE 8
Dosage regimen.

| Muscle/Site | Dosage (Unit Dose) |
|---|---|
| Lateral upper orbicularis oculi | 1 × UD |
| Medial upper orbicularis oculi muscle | 1 × UD |
| Lateral lower orbicularis oculi | 1 × UD |

The administration may be unilateral or bilateral as required based on the specific presentation.

As outlined above, Example 11 shows that the modified BoNT/A does not diffuse away from the injection site and highlights the exceptional safety profile of modified BoNT/A (SEQ ID NO: 6). Based on these findings, it is considered credible that much higher unit doses can be administered per muscle without resultant adverse effects. Furthermore, given the lack of systemic diffusion of the toxin, it is credible that up to 13-14× the higher unit doses can be administered without safety concerns.

Thus, a maximum total dosage administered is 82,500 pg. This is just over 13× greater than the maximum total dosage of Dysport® that can be administered during treatment of blepharospasm or hemifacial spasm without approaching toxic limits (a concern with conventional treatment regimens). Thus, the clinician is able to tailor treatment to the patient with the knowledge that a total dose 82,500 pg can be administered without any concern of toxicity, thereby allowing the treatment of additional muscles of the subject and/or ensuring each muscle and/or site thereof receives an effective dose.

### Example 13

### Dosage Regimen for Treating Typical Hemifacial Spasm Using a Modified BoNT/A (SEQ ID NO: 6)

Modified BoNT/A is provided as a lyophilised powder in a vial containing 36 ng of modified BoNT/A per vial. The lyophilised powder is reconstituted.

The unit dose (UD) is 240-8,000 pg (˜10-332.7 Units [measured by mouse LD₅₀]).

The disorder is treated by injection according to the following dosage regimen (Table 9):

TABLE 9
Dosage regimen.

| Muscle/Site | Dosage (Unit Dose) |
|---|---|
| Lateral upper orbicularis oculi | 1 × UD |
| Medial upper orbicularis oculi muscle | 1 × UD |
| Lateral lower orbicularis oculi | 1 × UD |

And also:

| | |
|---|---|
| orbicularis oris upper muscle | 1 × UD |
| orbicularis oris lower muscle | 1 × UD |
| zygomaticus major muscle | 1 × UD |
| zygomaticus minor muscle | 1 × UD |
| frontalis muscle | Up to 5 × UD |
| mentalis muscle | 1 × UD |
| platysma muscle | 1 × UD |
| corrugator muscle | 1 × UD |
| buccinator muscle | 1 × UD |
| masseter muscle | Up to 2 × UD |
| procerus muscle | 1 × UD |
| nasalis muscle | 1 × UD |
| levator palpebrae superiori muscle | 1 × UD |

A maximum total dosage administered is 82,500 pg. This is just over 13× greater than the maximum total dosage of Dysport® that can be administered during treatment of blepharospasm or hemifacial spasm without approaching toxic limits (a concern with conventional treatment regimens). Thus, the clinician is able to tailor treatment to the patient with the knowledge that a total dose 82,500 pg can be administered without any concern of toxicity, thereby allowing the treatment of additional muscles of the subject and/or ensuring each muscle and/or site thereof receives an effective dose.

### Example 14

Treatment of a Patient with Blepharospasm

Bill, aged 53, is diagnosed by his GP with blepharospasm. Modified BoNT/A (SEQ ID NO: 6) is administered by way of one or more unit doses (1× unit dose=5000 pg) to the following muscles indicated below

| | Dosage | Total |
|---|---|---|
| Muscle/Site | (Unit Dose) | Volume |
| Lateral upper orbicularis oculi | 1 × UD | 1 ml |
| Medial upper orbicularis oculi muscle | 1 × UD | 1 ml |
| Lateral lower orbicularis oculi | 1 × UD | 1 ml |

The total dose administered is 82,500 ng modified BoNT/A (SEQ ID NO: 6).

The blepharospasm is alleviated and, owing to the long duration of the modified BoNT/A, Bill does not require further treatment for greater than 9 months. Thus, Bill receives less frequent injections (e.g. per year) when compared to an equivalent subject administered an unmodified BoNT/A. Additionally, Bill does not exhibit any side-effects owing to the improved safety profile of the modified BoNT/A.

### Example 15

Treatment of a Patient with Typical Hemifacial Spasm

Jane, aged 45, is diagnosed by here GP with typical hemifacial spasm. Modified BoNT/A (SEQ ID NO: 6) is administered by way of one or more unit doses (1× unit dose=5000 pg) to the following muscles indicated below

| | Dosage | Total |
|---|---|---|
| Muscle/Site | (Unit Dose) | Volume |
| Lateral upper orbicularis oculi | 1 × UD | 1 ml |
| Medial upper orbicularis oculi muscle | 1 × UD | 1 ml |
| Lateral lower orbicularis oculi | 1 × UD | 1 ml |

And also:

| | | |
|---|---|---|
| orbicularis oris upper muscle | 1 × UD | 1 ml |
| orbicularis oris lower muscle | 1 × UD | 1 ml |
| zygomaticus major muscle | 1 × UD | 1 ml |
| zygomaticus minor muscle | 1 × UD | 1 ml |
| frontalis muscle | Up to 5 × UD | Up to 5 ml |
| mentalis muscle | 1 × UD | 1 ml |
| platysma muscle | 1 × UD | 1 ml |
| corrugator muscle | 1 × UD | 1 ml |
| buccinator muscle | 1 × UD | 1 ml |
| masseter muscle | Up to 2 × UD | Up to 2 ml |
| procerus muscle | 1 × UD | 1 ml |
| nasalis muscle | 1 × UD | 1 ml |
| levator palpebrae superiori muscle | 1 × UD | 1 ml |

The total dose administered is 82,500 ng modified BoNT/A (SEQ ID NO: 6). The hemifacial spasm is alleviated and, owing to the long duration of the modified BoNT/A, Jane does not require further treatment for greater than 9 months. Thus, Jane receives less frequent injections (e.g. per year) when compared to an equivalent subject administered an unmodified BoNT/A. Additionally, Jane does not exhibit any side-effects owing to the improved safety profile of the modified BoNT/A.

### Example 16

Treatment of a Patient with Blepharospasm

James, aged 50, is diagnosed by his GP with unilateral blepharospasm. Modified BoNT/A (SEQ ID NO: 6) is administered by way of one or more unit doses (1× unit dose=2500 pg) to the following muscles (of the eye that is affected by the unilateral blepharospasm) indicated below

| Muscle/Site | Dosage (Unit Dose) |
|---|---|
| Lateral upper orbicularis oculi | 2 × UD |
| Medial upper orbicularis oculi muscle | 1 × UD |
| Lateral lower orbicularis oculi | 1 × UD |

The total dose administered is 15,000 ng (to one eye) of modified BoNT/A (SEQ ID NO: 6).

The blepharospasm is alleviated and, owing to the long duration of the modified BoNT/A, James does not require further treatment for greater than 9 months. Thus, James receives less frequent injections (e.g. per year) when compared to an equivalent subject administered an unmodified BoNT/A. Additionally, James does not exhibit any side-effects owing to the improved safety profile of the modified BoNT/A.

### Example 17

Treatment of a Patient with Blepharospasm

Alice, aged 46, is diagnosed by here GP with unilateral blepharospasm. Modified BoNT/A (SEQ ID NO: 6) is administered by way of one or more unit doses (1× unit dose=4000 pg) to the following muscles (of the eye that is affected by the unilateral blepharospasm) indicated below

| Muscle/Site | Dosage (Unit Dose) |
|---|---|
| Lateral upper orbicularis oculi | 2 × UD |
| Medial upper orbicularis oculi muscle | 1 × UD |
| Lateral lower orbicularis oculi | 1 × UD |

The total dose administered is 24,000 ng (to one eye) of modified BoNT/A (SEQ ID NO: 6).

The blepharospasm is alleviated and, owing to the long duration of the modified BoNT/A, Alice does not require further treatment for greater than 9 months. Thus, Alice receives less frequent injections (e.g. per year) when compared to an equivalent subject administered an unmodified BoNT/A. Additionally, Alice does not exhibit any side-effects owing to the improved safety profile of the modified BoNT/A.

### Example 18

Treatment of a Patient with Blepharospasm

Peter, aged 45, is diagnosed by his GP with bilateral blepharospasm. Modified BoNT/A (SEQ ID NO: 6) is administered by way of one or more unit doses (1× unit dose=2500 pg) to the following muscles (at both eyes that are affected by the bilateral blepharospasm) indicated below

| Muscle/Site | Dosage (Unit Dose) |
|---|---|
| Lateral upper orbicularis oculi | 2 × UD |
| Medial upper orbicularis oculi muscle | 1 × UD |
| Lateral lower orbicularis oculi | 1 × UD |

The total dose administered is 15,000 ng to each eye (thus 30,000 pg across both eyes) of modified BoNT/A (SEQ ID NO: 6).

The blepharospasm is alleviated and, owing to the long duration of the modified BoNT/A, Peter does not require further treatment for greater than 9 months. Thus, Peter receives less frequent injections (e.g. per year) when compared to an equivalent subject administered an unmodified BoNT/A. Additionally, Peter does not exhibit any side-effects owing to the improved safety profile of the modified BoNT/A.

### Example 19

Treatment of a Patient with Blepharospasm

Greta, aged 41, is diagnosed by here GP with bilateral blepharospasm. Modified BoNT/A (SEQ ID NO: 6) is administered by way of one or more unit doses (1× unit dose=4000 pg) to the following muscles (of both eyes that are affected by the bilateral blepharospasm) indicated below

| Muscle/Site | Dosage (Unit Dose) |
|---|---|
| Lateral upper orbicularis oculi | 2 × UD |
| Medial upper orbicularis oculi muscle | 1 × UD |
| Lateral lower orbicularis oculi | 1 × UD |

The total dose administered is 24,000 ng to each eye (thus 48,000 ng across both eyes) of modified BoNT/A (SEQ ID NO: 6).

The blepharospasm is alleviated and, owing to the long duration of the modified BoNT/A, Greta does not require further treatment for greater than 9 months. Thus, Greta receives less frequent injections (e.g. per year) when compared to an equivalent subject administered an unmodified BoNT/A. Additionally, Greta does not exhibit any side-effects owing to the improved safety profile of the modified BoNT/A.

### Example 20

Treatment of a Patient with Blepharospasm

A patient presents with bilateral blepharospasm. A GP prescribes a total of 12 injections of modified BoNT/A (each injection having the same unit dose), 6 per eye. The precise locations are based on the pattern of the patient's blepharospasm. The injection regimen involves a tailored regimen of 6 injections per are eye positioned across: up to six different injection sites of the upper orbicularis oculi muscle across the following sites:
the medial upper orbicularis oculi muscle
the superior orbital orbicularis oculi muscle;
the lateral upper orbicularis oculi muscle
the outer orbital orbicularis oculi muscle;
the medial upper pretarsal orbital orbicularis oculi muscle; and
the lateral upper pretarsal orbital orbicularis oculi muscle; and/or
   up to two different injection sites of the lower orbicularis oculi muscle proximal across the following sites;
the medial lower orbicularis oculi; and
the lateral lower orbicularis oculi muscle; and/or
   up to two different injection sites selected from:
two different injection sites of the corrugator proximal to the first eye of the subject; and
one site on the procerus proximal to the first eye of the subject.

The blepharospasm is alleviated and, owing to the long duration of the modified BoNT/A, the patient does not require further treatment for greater than 9 months. Thus, the patient receives less frequent injections (e.g. per year) when compared to an equivalent subject administered an unmodified BoNT/A. Additionally, the patient does not exhibit any side-effects owing to the improved safety profile of the modified BoNT/A.

### Example 21

Safety & Efficacy of Modified BoNT/a (SEQ ID NO: 6 Converted into a Di-Chain Form) in Humans

SEQ ID NO: 6 (converted into a di-chain form) was administered to human subjects by way of intramuscular injection. Subjects were administered 2× of a 15,000 pg unit dose (i.e. 30,000 pg total), 2× of a 25,000 pg unit dose (i.e. 50,000 pg total), or 2× of a 36,000 pg unit dose (i.e. 72,000 pg total) of modified BoNT/A (SEQ ID NO: 6 converted into a di-chain form).

Results showed that all unit doses of modified BoNT/A tested were effective at muscle paralysis, safely tolerated, and no adverse effects were observed, despite the exceptionally high dosage per muscle (e.g. for the 25,000 pg and 36,000 pg unit dose). This shows that the modified BoNT/A does not diffuse away from the injection site and highlights the exceptional safety profile of modified BoNT/A (SEQ ID NO: 6 converted into a di-chain form).

The unit doses and the total doses described herein in the context or treating a facial (incl. blepharospasm, typical hemifacial spasm, atypical hemifacial spasm) are well within the dose range shown to be safe and efficacious for this advantageous modified BoNT/A molecules described herein, providing clinicians with flexibility in terms of treatment options that includes utilisation of the advantageous properties and exceptional safety profile of modified BoNT/A (such as SEQ ID NO: 6 converted into a di-chain form).

### Clauses

1. A modified botulinum neurotoxin A (BoNT/A) for use in a method of treating a disorder affecting an eyelid muscle of a subject, the method comprising:
administering a single unit dose of the modified BoNT/A to the lateral upper orbicularis oculi muscle proximal to a first eye of the subject;
administering a single unit dose of the modified BoNT/A to the medial upper orbicularis oculi muscle proximal to the first eye of the subject; and
administering a single unit dose of the modified BoNT/A to the lateral lower orbicularis oculi muscle proximal to the first eye of the subject,
wherein the single unit dose of the modified BoNT/A is at least 240 pg (preferably 240 pg to 8,000 pg) of modified BoNT/A,
wherein the total dose administered during the treatment is up to 24,000 pg of the modified BoNT/A, and
wherein the modified BoNT/A comprises a BoNT/A light-chain and translocation domain, and a BoNT/B receptor binding domain (H_{C} domain).
   2. The modified BoNT/A for use according to clause 1, wherein the single unit dose of the modified BoNT/A is 240 pg to 4,800 pg of modified BoNT/A.
   3. The modified BoNT/A for use according to clause 1 or 2, wherein the single unit dose of the modified BoNT/A is 240 pg to 4,000 pg of modified BoNT/A.
   4. The modified BoNT/A for use according to any one of the preceding clauses, wherein the single unit dose of the modified BoNT/A is 240 pg to 2,400 pg of modified BoNT/A.
   5. The modified BoNT/A for use according to any one of the preceding clauses, wherein the single unit dose of the modified BoNT/A is 240 pg to 2,000 pg of modified BoNT/A.
   6. The modified BoNT/A for use according to any one of the preceding clauses, wherein the single unit dose (e.g. the lower limit of the single unit dose) is at least 500 pg of modified BoNT/A.
   7. The modified BoNT/A for use according to any one of the preceding clauses, wherein the single unit dose (e.g. the lower limit of the single unit dose) is at least 1,000 pg of modified BoNT/A.
   8. A modified botulinum neurotoxin A (BoNT/A) for use in a method of treating a disorder affecting an eyelid muscle of a subject, the method comprising:
administering a single unit dose of the modified BoNT/A to the lateral upper orbicularis oculi muscle proximal to a first eye of the subject;
administering a single unit dose of the modified BoNT/A to the medial upper orbicularis oculi muscle proximal to the first eye of the subject; and
administering a single unit dose of the modified BoNT/A to the lateral lower orbicularis oculi muscle proximal to the first eye of the subject,
wherein the single unit dose of the modified BoNT/A is at least 10 Units (U) (preferably 10 U to 332.7 U) of modified BoNT/A, wherein 1 Unit is an amount of the modified BoNT/A that corresponds to the calculated median lethal dose (LD₅₀) in mice,
wherein the total dose administered during the treatment is up to 998 U of the modified BoNT/A, and
wherein the modified BoNT/A comprises a BoNT/A light-chain and translocation domain, and a BoNT/B receptor binding domain (H_{C} domain).
   9. The modified BoNT/A for use according to clause 8, wherein the single unit dose of the modified BoNT/A is 10 U to 199.6 U of modified BoNT/A.
   10. The modified BoNT/A for use according to clause 8 or 9, wherein the single unit dose of the modified BoNT/A is 10 U to 166.3 U of modified BoNT/A.
   11. The modified BoNT/A for use according to any one of clauses 8-10, wherein the single unit dose of the modified BoNT/A is 10 U to 99.8 U of modified BoNT/A.
   12. The modified BoNT/A for use according to any one of clauses 8-11, wherein the single unit dose of the modified BoNT/A is 10 U to 83.17 U of modified BoNT/A.
   13. The modified BoNT/A for use according to any one of clauses 8-12, wherein the single unit dose (e.g. the lower limit of the single unit dose) is at least 21 U of modified BoNT/A.
   14. The modified BoNT/A for use according to any one clauses 8-13, wherein the single unit dose (e.g. the lower limit of the single unit dose) is at least 42 U of modified BoNT/A.
   15. The modified BoNT/A for use according to any one of the preceding clauses, wherein the modified BoNT/A comprises a polypeptide sequence having at least 70% sequence identity to SEQ ID NO: 14.
   16. A modified botulinum neurotoxin A (BoNT/A) for use in a method of treating a disorder affecting an eyelid muscle of a subject, the method comprising:
administering a single unit dose of the modified BoNT/A to the lateral upper orbicularis oculi muscle proximal to a first eye of the subject;
administering a single unit dose of the modified BoNT/A to the medial upper orbicularis oculi muscle proximal to the first eye of the subject; and
administering a single unit dose of the modified BoNT/A to the lateral lower orbicularis oculi muscle proximal to the first eye of the subject,
wherein the single unit dose of the modified BoNT/A is at least 84 pg (preferably 84 pg to 666.7 pg) of modified BoNT/A,
wherein the total dose administered during the treatment is up to 2,000 pg of the modified BoNT/A, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   (i) substitution of an acidic surface exposed amino acid residue with a basic amino acid residue;
   (ii) substitution of an acidic surface exposed amino acid residue with an uncharged amino acid residue;
   (iii) substitution of an uncharged surface exposed amino acid residue with a basic amino acid residue;
   (iv) insertion of a basic amino acid residue; and
   (v) deletion of an acidic surface exposed amino acid residue.
      17. The modified BoNT/A for use according to clause 16, wherein the single unit dose of the modified BoNT/A is 84 pg to 400 pg of modified BoNT/A.
      18. The modified BoNT/A for use according to clause 16 or 17, wherein the single unit dose of the modified BoNT/A is 84 pg to 333.3 pg of modified BoNT/A.
      19. The modified BoNT/A for use according to any one of clauses 16-18, wherein the single unit dose of the modified BoNT/A is 84 pg to 200 pg of modified BoNT/A.
      20. The modified BoNT/A for use according to any one of clauses 16-19, wherein the single unit dose of the modified BoNT/A is 84 pg to 166.7 pg of modified BoNT/A.
      21. The modified BoNT/A for use according to any one of clauses 16-20, wherein the single unit dose (e.g. the lower limit of the single unit dose) is at least 100 pg of modified BoNT/A.
      22. A modified botulinum neurotoxin A (BoNT/A) for use in a method of treating a disorder affecting an eyelid muscle of a subject, the method comprising:
administering a single unit dose of the modified BoNT/A to the lateral upper orbicularis oculi muscle proximal to a first eye of the subject;
administering a single unit dose of the modified BoNT/A to the medial upper orbicularis oculi muscle proximal to the first eye of the subject; and
administering a single unit dose of the modified BoNT/A to the lateral lower orbicularis oculi muscle proximal to the first eye of the subject,
wherein the single unit dose of the modified BoNT/A is at least 10 U (preferably 10 U to 79 U) of modified BoNT/A, wherein 1 Unit is an amount of the modified BoNT/A that corresponds to the calculated median lethal dose (LD₅₀) in mice,
wherein the total dose administered during the treatment is up to 237 U of the modified BoNT/A, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   (i) substitution of an acidic surface exposed amino acid residue with a basic amino acid residue;
   (ii) substitution of an acidic surface exposed amino acid residue with an uncharged amino acid residue;
   (iii) substitution of an uncharged surface exposed amino acid residue with a basic amino acid residue;
   (iv) insertion of a basic amino acid residue; and
   (v) deletion of an acidic surface exposed amino acid residue.
      23. The modified BoNT/A for use according to clause 22, wherein the single unit dose of the modified BoNT/A is 10 U to 47.4 U of modified BoNT/A.
      24. The modified BoNT/A for use according to clause 22 or 23, wherein the single unit dose of the modified BoNT/A is 10 U to 39.5 U of modified BoNT/A.
      25. The modified BoNT/A for use according to any one of clauses 22-24, wherein the single unit dose of the modified BoNT/A is 10 U to 23.7 U of modified BoNT/A.
      26. The modified BoNT/A for use according to any one of clauses 22-25, wherein the single unit dose of the modified BoNT/A is 10 U to 19.75 U of modified BoNT/A.
      27. The modified BoNT/A for use according to any one of clauses 22-26, wherein the single unit dose (e.g. the lower limit of the single unit dose) is at least 12 U of modified BoNT/A.
      28. The modified BoNT/A for use according to any one of clauses 16-27, wherein said modification comprises (preferably consists of) a modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 or THR 1277, and wherein the modified BoNT/A is encoded by a nucleic acid sequence having at least 70% sequence identity to a nucleic acid sequence selected from SEQ ID NOs: 3, 5, 7, and 9, and/or comprises a polypeptide sequence having at least 70% sequence identity to a polypeptide sequence selected from SEQ ID NOs: 4, 6, 8, and 10, preferably wherein said modification comprises (preferably consists of) a modification at one or more amino acid residue(s) selected from: ASN 886, ASN 930, SER 955, GLN 991, ASN 1026, ASN 1052, and GLN 1229, and wherein the modified BoNT/A is encoded by a nucleic acid sequence having at least 70% sequence identity to SEQ ID NO: 3, and/or comprises a polypeptide sequence having at least 70% sequence identity to an amino acid sequence selected from SEQ ID NO: 4.
      29. The modified BoNT/A for use according any to any one of clauses 16-28, wherein the modification is a substitution, preferably a substitution with lysine or arginine.
      30. The modified BoNT/A for use according to any one of the preceding clauses, wherein the modified BoNT/A has a Safety Ratio of greater than 7, wherein the Safety Ratio is calculated as: dose of toxin required for −10% bodyweight change measured as pg/mouse divided by DAS ED₅₀ measured as pg/mouse, wherein ED₅₀=dose required to produce a DAS score of 2.
      31. The modified BoNT/A for use according to any one of clauses 1, 3-8, 10-16, 18-22 or 24-30, wherein the method further comprises:
administering a single unit dose of the modified BoNT/A to the lateral upper orbicularis oculi muscle proximal to a second eye of the subject;
administering a single unit dose of the modified BoNT/A to the medial upper orbicularis oculi muscle proximal to the second eye of the subject; and
administering a single unit dose of the modified BoNT/A to the lateral lower orbicularis oculi muscle proximal to the second eye of the subject.
   32. The modified BoNT/A for use according to any one of the preceding clauses, wherein the disorder affecting an eyelid muscle of a subject is blepharospasm.
   33. The modified BoNT/A for use according to any one of the preceding clauses, further comprising administering a single unit dose of the modified BoNT/A to the medial lower orbicularis oculi muscle proximal to the eye of the subject, wherein the total dose of modified BoNT/A administered during the treatment does not exceed that stated (e.g. in the preceding clause).
   34. The modified BoNT/A for use according to any one of the preceding clauses, further comprising administering at least a single unit dose (e.g. two unit doses) of the modified BoNT/A to the frontalis muscle proximal to the eye of the subject, wherein the total dose of modified BoNT/A administered during the treatment does not exceed that stated (e.g. in the preceding clause).
   35. The modified BoNT/A for use according to any one of the preceding clauses, further comprising administering at least a single unit dose (e.g. two unit doses) of the modified BoNT/A to the corrugator muscle proximal to the eye of the subject, wherein the total dose of modified BoNT/A administered during the treatment does not exceed that stated (e.g. in the preceding clause).
   36. The modified BoNT/A for use according to any one of the preceding clauses, wherein the disorder affecting an eyelid muscle of a subject is hemifacial spasm.
   37. The modified BoNT/A for use according to clause 36, further comprising administering the modified BoNT/A to one or more muscles selected from: the orbicularis oris (e.g. the orbicularis oris upper and/or the orbicularis oris lower); the zygomaticus (e.g. zygomaticus major); the nasalis; the mentalis; the platysma; the frontalis; the corrugator; the buccinator; the masseter; the procerus; and the lateral canthus, wherein the total dose of modified BoNT/A administered during the treatment does not exceed that stated (e.g. in the preceding clause).
   38. The modified BoNT/A for use according to any one of the preceding clauses, wherein the BoNT/A is administered subcutaneously, preferably by subcutaneous injection.
   39. The modified BoNT/A for use according to any one of clauses 1-38, wherein the BoNT/A is administered intramuscularly, preferably by intramuscular injection.
   40. The modified BoNT/A for use according to any one of the preceding clauses, wherein the modified BoNT/A is administered by way of a single unit dose per injection site.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A modified botulinum neurotoxin A (BoNT/A) for use in a method of treating blepharospasm in a subject, wherein the modified BoNT/A is administered by intramuscular injection at a plurality of sites of the face of the subject, the method comprising:
a) administering a unit dose of the modified BoNT/A per injection site at up to six different injection sites of the upper orbicularis oculi muscle proximal to a first eye of the subject, wherein said up to six different injection sites are selected from:
i) the medial upper preseptal orbicularis oculi muscle;
ii) the superior orbital orbicularis oculi muscle;
iii) the lateral upper preseptal orbicularis oculi muscle;
iv) the outer orbital orbicularis oculi muscle;
v) the medial upper pretarsal orbital orbicularis oculi muscle; and
vi) the lateral upper pretarsal orbital orbicularis oculi muscle; and/or
b) administering a unit dose of the modified BoNT/A per injection site at up to two different injection sites of the lower orbicularis oculi muscle proximal to the first eye of the subject, wherein said up to two different injection sites are selected from:
i) the medial lower orbicularis oculi muscle; and
ii) the lateral lower orbicularis oculi muscle; and/or
c) administering a unit dose of the modified BoNT/A per injection site at up to two different injection sites selected from:
i) two different injection sites of the corrugator proximal to the first eye of the subject; and
ii) one site on the procerus proximal to the first eye of the subject;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a BoNT/A light-chain and translocation domain (H_{N}), and a BoNT/B receptor binding domain (H_{C} domain).

2. A method of treating blepharospasm in a subject, the method comprising administering a modified botulinum neurotoxin A (BoNT/A) by intramuscular injection at a plurality of sites of the face of the subject, the method comprising:
a) administering a unit dose of the modified BoNT/A per injection site at up to six different injection sites of the upper orbicularis oculi muscle proximal to a first eye of the subject, wherein said up to six different injection sites are selected from:
i) the medial upper preseptal orbicularis oculi muscle;
ii) the superior orbital orbicularis oculi muscle;
iii) the lateral upper preseptal orbicularis oculi muscle;
iv) the outer orbital orbicularis oculi muscle;
v) the medial upper pretarsal orbital orbicularis oculi muscle; and
vi) the lateral upper pretarsal orbital orbicularis oculi muscle; and/or
b) administering a unit dose of the modified BoNT/A per injection site at up to two different injection sites of the lower orbicularis oculi muscle proximal to the first eye of the subject, wherein said up to two different injection sites are selected from:
i) the medial lower orbicularis oculi muscle; and
ii) the lateral lower orbicularis oculi muscle; and/or
c) administering a unit dose of the modified BoNT/A per injection site at up to two different injection sites selected from:
i) two different injection sites of the corrugator proximal to the first eye of the subject; and
ii) one site on the procerus proximal to the first eye of the subject;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a BoNT/A light-chain and translocation domain (H_{N}), and a BoNT/B receptor binding domain (H_{C} domain).

3. Use of a modified botulinum neurotoxin A (BoNT/A) in the manufacture of a medicament for treating typical hemifacial spasm, wherein the modified BoNT/A is administered by intramuscular injection at a plurality of sites of the face of the subject, the treatment comprising:
(a) administering a unit dose of the modified BoNT/A per injection site at up to six different injection sites of the upper orbicularis oculi muscle proximal to a first eye of the subject, wherein said up to six different injection sites are selected from:
(i) the medial upper preseptal orbicularis oculi muscle;
(ii) the superior orbital orbicularis oculi muscle;
(iii) the lateral upper preseptal orbicularis oculi muscle;
(iv) the outer orbital orbicularis oculi muscle;
(v) the medial upper pretarsal orbital orbicularis oculi muscle; and
(vi) the lateral upper pretarsal orbital orbicularis oculi muscle; and/or
(b) administering a unit dose of the modified BoNT/A per injection site at up to two different injection sites of the lower orbicularis oculi muscle proximal to the first eye of the subject, wherein said up to two different injection sites are selected from:
(i) the medial lower orbicularis oculi muscle; and
(ii) the lateral lower orbicularis oculi muscle; and/or
(c) administering a unit dose of the modified BoNT/A per injection site at up to two different injection sites selected from:
(i) two different injection sites of the corrugator proximal to the first eye of the subject; and
(ii) one site on the procerus proximal to the first eye of the subject;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a BoNT/A light-chain and translocation domain (H_{N}), and a BoNT/B receptor binding domain (H_{C} domain).

4. The modified BoNT/A for use, the method or the use according to any one of claims the preceding claims, wherein the modified BoNT/A is administered by intramuscular injection at at least six different sites of the face of the subject.

5. The modified BoNT/A for use, the method or the use according to any one of the preceding claims, wherein the method for treating blepharospasm further comprises:
(a) administering a unit dose of the modified BoNT/A per injection site at up to six different injection sites of the upper orbicularis oculi muscle proximal to a second eye of the subject, wherein said up to six different injection sites are selected from:
(i) the medial upper preseptal orbicularis oculi muscle;
(ii) the superior orbital orbicularis oculi muscle;
(iii) the lateral upper preseptal orbicularis oculi muscle;
(iv) the outer orbital orbicularis oculi muscle;
(v) the medial upper pretarsal orbital orbicularis oculi muscle; and
(vi) the lateral upper pretarsal orbital orbicularis oculi muscle; and/or
(b) administering a unit dose of the modified BoNT/A per injection site at up to two different injection sites of the lower orbicularis oculi muscle proximal to the second eye of the subject, wherein said up to two different injection sites are selected from:
(i) the medial lower orbicularis oculi muscle; and
(ii) the lateral lower orbicularis oculi muscle; and/or
(c) administering a unit dose of the modified BoNT/A per injection site at up to two different injection sites selected from:
(i) two different injection sites of the corrugator proximal to the second eye of the subject; and
(ii) one site on the procerus proximal to the second eye of the subject.

6. The modified BoNT/A for use, the method or the use according to claim 5, wherein the modified BoNT/A is administered by intramuscular injection at at least six different sites per eye of the subject.

7. The modified BoNT/A for use, the method or the use according to any one of the preceding claims, wherein the method comprises:
(a) administering a unit dose of the modified BoNT/A to the lateral upper preseptal orbicularis oculi muscle proximal to a first eye of the subject;
(b) administering a unit dose of the modified BoNT/A to the medial upper preseptal orbicularis oculi muscle proximal to the first eye of the subject; and
(c) administering a unit dose of the modified BoNT/A to the lateral lower orbicularis oculi muscle (preferably the lateral lower preseptal orbicularis oculi muscle) proximal to the first eye of the subject.

8. A modified botulinum neurotoxin A (BoNT/A) for use in a method of treating typical hemifacial spasm, wherein the modified BoNT/A is administered by intramuscular injection at a plurality of sites of the face of the subject, the method comprising:
(a) administering a unit dose of the modified BoNT/A per injection site at up to six different injection sites of the upper orbicularis oculi muscle proximal to a first eye of the subject, wherein said up to six different injection sites are selected from:
(i) the medial upper preseptal orbicularis oculi muscle;
(ii) the superior orbital orbicularis oculi muscle;
(iii) the lateral upper preseptal orbicularis oculi muscle;
(iv) the outer orbital orbicularis oculi muscle;
(v) the medial upper pretarsal orbital orbicularis oculi muscle; and
(vi) the lateral upper pretarsal orbital orbicularis oculi muscle; and/or
(b) administering a unit dose of the modified BoNT/A per injection site at up to two different injection sites of the lower orbicularis oculi muscle proximal to the first eye of the subject, wherein said up to two different injection sites are selected from:
(i) the medial lower orbicularis oculi muscle; and
(ii) the lateral lower orbicularis oculi muscle; and/or
(c) administering a unit dose of the modified BoNT/A per injection site at up to two different injection sites selected from:
(i) two different injection sites of the corrugator proximal to the first eye of the subject; and
(ii) one site on the procerus proximal to the first eye of the subject; and/or
(d) administering one or more unit dose of the modified BoNT/A to one or more further muscles affected by said hemifacial spasm in accordance with the following dosage regimen:
(i) one unit dose to an orbicularis oris upper muscle;
(ii) one unit dose to an orbicularis oris lower muscle;
(iii) one unit dose to a zygomaticus major muscle;
(iv) one unit dose to a zygomaticus minor muscle;
(v) up to five unit doses (preferably one unit dose) to a frontalis muscle;
(vi) one unit dose to a mentalis muscle;
(vii) one unit dose to a platysma muscle;
(viii) up to two unit doses (preferably one unit dose) to a corrugator muscle;
(ix) one unit dose to a buccinator muscle;
(x) up to two unit doses (preferably one unit dose) to a masseter muscle;
(xi) one unit dose to a procerus muscle;
(xii) one unit dose to a nasalis muscle; and/or
(xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

9. A method of treating typical hemifacial spasm, the method comprising administering a modified BoNT/A by intramuscular injection at a plurality of sites of the face of the subject, the method comprising:
(a) administering a unit dose of the modified BoNT/A per injection site at up to six different injection sites of the upper orbicularis oculi muscle proximal to a first eye of the subject, wherein said up to six different injection sites are selected from:
(i) the medial upper preseptal orbicularis oculi muscle;
(ii) the superior orbital orbicularis oculi muscle;
(iii) the lateral upper preseptal orbicularis oculi muscle;
(iv) the outer orbital orbicularis oculi muscle;
(v) the medial upper pretarsal orbital orbicularis oculi muscle; and
(vi) the lateral upper pretarsal orbital orbicularis oculi muscle; and/or
(b) administering a unit dose of the modified BoNT/A per injection site at up to two different injection sites of the lower orbicularis oculi muscle proximal to the first eye of the subject, wherein said up to two different injection sites are selected from:
(i) the medial lower orbicularis oculi muscle; and
(ii) the lateral lower orbicularis oculi muscle; and/or
(c) administering a unit dose of the modified BoNT/A per injection site at up to two different injection sites selected from:
(i) two different injection sites of the corrugator proximal to the first eye of the subject; and
(ii) one site on the procerus proximal to the first eye of the subject; and/or
(d) administering one or more unit dose of the modified BoNT/A to one or more further muscles affected by said hemifacial spasm in accordance with the following dosage regimen:
(i) one unit dose to an orbicularis oris upper muscle;
(ii) one unit dose to an orbicularis oris lower muscle;
(iii) one unit dose to a zygomaticus major muscle;
(iv) one unit dose to a zygomaticus minor muscle;
(v) up to five unit doses (preferably one unit dose) to a frontalis muscle;
(vi) one unit dose to a mentalis muscle;
(vii) one unit dose to a platysma muscle;
(viii) up to two unit doses (preferably one unit dose) to a corrugator muscle;
(ix) one unit dose to a buccinator muscle;
(x) up to two unit doses (preferably one unit dose) to a masseter muscle;
(xi) one unit dose to a procerus muscle;
(xii) one unit dose to a nasalis muscle; and/or
(xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

10. Use of a modified botulinum neurotoxin A (BoNT/A) in the manufacture of a medicament for treating typical hemifacial spasm, wherein the modified BoNT/A is administered by intramuscular injection at a plurality of sites of the face of the subject, the treatment comprising:
(a) administering a unit dose of the modified BoNT/A per injection site at up to six different injection sites of the upper orbicularis oculi muscle proximal to a first eye of the subject, wherein said up to six different injection sites are selected from:
(i) the medial upper preseptal orbicularis oculi muscle;
(ii) the superior orbital orbicularis oculi muscle;
(iii) the lateral upper preseptal orbicularis oculi muscle;
(iv) the outer orbital orbicularis oculi muscle;
(v) the medial upper pretarsal orbital orbicularis oculi muscle; and
(vi) the lateral upper pretarsal orbital orbicularis oculi muscle; and/or
(b) administering a unit dose of the modified BoNT/A per injection site at up to two different injection sites of the lower orbicularis oculi muscle proximal to the first eye of the subject, wherein said up to two different injection sites are selected from:
(i) the medial lower orbicularis oculi muscle; and
(ii) the lateral lower orbicularis oculi muscle; and/or
(c) administering a unit dose of the modified BoNT/A per injection site at up to two different injection sites selected from:
(i) two different injection sites of the corrugator proximal to the first eye of the subject; and
(ii) one site on the procerus proximal to the first eye of the subject; and/or
(d) administering one or more unit dose of the modified BoNT/A to one or more further muscles affected by said hemifacial spasm in accordance with the following dosage regimen:
(i) one unit dose to an orbicularis oris upper muscle;
(ii) one unit dose to an orbicularis oris lower muscle;
(iii) one unit dose to a zygomaticus major muscle;
(iv) one unit dose to a zygomaticus minor muscle;
(v) up to five unit doses (preferably one unit dose) to a frontalis muscle;
(vi) one unit dose to a mentalis muscle;
(vii) one unit dose to a platysma muscle;
(viii) up to two unit doses (preferably one unit dose) to a corrugator muscle;
(ix) one unit dose to a buccinator muscle;
(x) up to two unit doses (preferably one unit dose) to a masseter muscle;
(xi) one unit dose to a procerus muscle;
(xii) one unit dose to a nasalis muscle; and/or
(xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

11. The modified BoNT/A for use, the method or the use according to any one of the preceding claims, wherein the modified BoNT/A is administered by intramuscular injection at at least six different sites of the face of the subject.

12. The modified BoNT/A for use, the method or the use according to any one of the preceding claims, wherein the method comprises:
(a) administering a unit dose of the modified BoNT/A to the lateral upper preseptal orbicularis oculi muscle proximal to a first eye of the subject;
(b) administering a unit dose of the modified BoNT/A to the medial upper preseptal orbicularis oculi muscle proximal to the first eye of the subject; and
(c) administering a unit dose of the modified BoNT/A to the lateral lower orbicularis oculi muscle (preferably the lateral lower preseptal orbicularis oculi muscle) proximal to the first eye of the subject.

13. A modified botulinum neurotoxin A (BoNT/A) for use in treating atypical hemifacial spasm, wherein the modified BoNT/A is administered by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
(i) one unit dose to a zygomaticus major muscle;
(ii) one unit dose to a zygomaticus minor muscle;
(iii) up to five unit doses (preferably one unit dose) to a frontalis muscle;
(iv) one unit dose to a mentalis muscle;
(v) one unit dose to a platysma muscle;
(vi) one unit dose to a buccinator muscle;
(vii) up to two unit doses (preferably one unit dose) to a masseter muscle;
(viii) one unit dose to a nasalis muscle;
(ix) one unit dose to a levator palpebrae superiori muscle; and/or
c) optionally administering one or more unit dose of the modified BoNT/A to one or more further muscles affected by said hemifacial spasm in accordance with the following dosage regimen:
a unit dose per injection site at up to six different injection sites of the upper orbicularis oculi muscle selected from:
(i) the medial upper preseptal orbicularis oculi muscle;
(ii) the superior orbital orbicularis oculi muscle;
(iii) the lateral upper preseptal orbicularis oculi muscle;
(iv) the outer orbital orbicularis oculi muscle;
(v) the medial upper pretarsal orbital orbicularis oculi muscle; and
(vi) the lateral upper pretarsal orbital orbicularis oculi muscle; and/or
a unit dose per injection site at up to two different injection sites of the lower orbicularis oculi muscle selected from:
(i) the medial lower orbicularis oculi muscle; and
(ii) the lateral lower orbicularis oculi muscle; and/or
a unit dose per injection site at up to two different injection sites selected from:
(i) two different injection sites of the corrugator; and
(ii) one site on the procerus;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

14. A method of treating atypical hemifacial spasm, the method comprising administering a modified BoNT/A by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
(i) one unit dose to a zygomaticus major muscle;
(ii) one unit dose to a zygomaticus minor muscle;
(iii) up to five unit doses (preferably one unit dose) to a frontalis muscle;
(iv) one unit dose to a mentalis muscle;
(v) one unit dose to a platysma muscle;
(vi) one unit dose to a buccinator muscle;
(vii) up to two unit doses (preferably one unit dose) to a masseter muscle;
(viii) one unit dose to a nasalis muscle;
(ix) one unit dose to a levator palpebrae superiori muscle; and/or
c) optionally administering one or more unit dose of the modified BoNT/A to one or more further muscles affected by said hemifacial spasm in accordance with the following dosage regimen:
a unit dose per injection site at up to six different injection sites of the upper orbicularis oculi muscle selected from:
(i) the medial upper preseptal orbicularis oculi muscle;
(ii) the superior orbital orbicularis oculi muscle;
(iii) the lateral upper preseptal orbicularis oculi muscle;
(iv) the outer orbital orbicularis oculi muscle;
(v) the medial upper pretarsal orbital orbicularis oculi muscle; and
(vi) the lateral upper pretarsal orbital orbicularis oculi muscle; and/or
a unit dose per injection site at up to two different injection sites of the lower orbicularis oculi muscle selected from:
(i) the medial lower orbicularis oculi muscle; and
(ii) the lateral lower orbicularis oculi muscle; and/or
a unit dose per injection site at up to two different injection sites selected from:
(i) two different injection sites of the corrugator; and
(ii) one site on the procerus;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

15. Use of a modified botulinum neurotoxin A (BoNT/A) in the manufacture of a medicament for treating atypical hemifacial spasm, wherein the modified BoNT/A is administered by intramuscular injection at a plurality of sites of the face of the subject, the treatment comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
(i) one unit dose to a zygomaticus major muscle;
(ii) one unit dose to a zygomaticus minor muscle;
(iii) up to five unit doses (preferably one unit dose) to a frontalis muscle;
(iv) one unit dose to a mentalis muscle;
(v) one unit dose to a platysma muscle;
(vi) one unit dose to a buccinator muscle;
(vii) up to two unit doses (preferably one unit dose) to a masseter muscle;
(viii) one unit dose to a nasalis muscle;
(ix) one unit dose to a levator palpebrae superiori muscle; and/or
c) optionally administering one or more unit dose of the modified BoNT/A to one or more further muscles affected by said hemifacial spasm in accordance with the following dosage regimen:
a unit dose per injection site at up to six different injection sites of the upper orbicularis oculi muscle selected from:
(i) the medial upper preseptal orbicularis oculi muscle;
(ii) the superior orbital orbicularis oculi muscle;
(iii) the lateral upper preseptal orbicularis oculi muscle;
(iv) the outer orbital orbicularis oculi muscle;
(v) the medial upper pretarsal orbital orbicularis oculi muscle; and
(vi) the lateral upper pretarsal orbital orbicularis oculi muscle; and/or
a unit dose per injection site at up to two different injection sites of the lower orbicularis oculi muscle selected from:
(i) the medial lower orbicularis oculi muscle; and
(ii) the lateral lower orbicularis oculi muscle; and/or
a unit dose per injection site at up to two different injection sites selected from:
(i) two different injection sites of the corrugator; and
(ii) one site on the procerus;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

16. A modified botulinum neurotoxin A (BoNT/A) for use in a method of treating blepharospasm in a subject, wherein the modified BoNT/A is administered by intramuscular injection at a plurality of sites of the face of the subject, the method comprising:
a) administering a unit dose of the modified BoNT/A to the lateral upper orbicularis oculi muscle proximal to a first eye of the subject;
b) administering a unit dose of the modified BoNT/A to the medial upper orbicularis oculi muscle proximal to the first eye of the subject; and
c) administering a unit dose of the modified BoNT/A to the lateral lower orbicularis oculi muscle proximal to the first eye of the subject,
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a BoNT/A light-chain and translocation domain (H_{N}), and a BoNT/B receptor binding domain (H_{C} domain).

17. A method of treating blepharospasm in a subject, the method comprising administering a modified BoNT/A by intramuscular injection at a plurality of sites of the face of the subject, the method comprising:
a) administering a unit dose of the modified BoNT/A to the lateral upper orbicularis oculi muscle proximal to a first eye of the subject;
b) administering a unit dose of the modified BoNT/A to the medial upper orbicularis oculi muscle proximal to the first eye of the subject; and
c) administering a unit dose of the modified BoNT/A to the lateral lower orbicularis oculi muscle proximal to the first eye of the subject,
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a BoNT/A light-chain and translocation domain (H_{N}), and a BoNT/B receptor binding domain (H_{C} domain).

18. Use of a modified botulinum neurotoxin A (BoNT/A) in the manufacture of a medicament for treating blepharospasm in a subject, wherein the modified BoNT/A is administered by intramuscular injection at a plurality of sites of the face of the subject, the treatment comprising:
a) administering a unit dose of the modified BoNT/A to the lateral upper orbicularis oculi muscle proximal to a first eye of the subject;
b) administering a unit dose of the modified BoNT/A to the medial upper orbicularis oculi muscle proximal to the first eye of the subject; and
c) administering a unit dose of the modified BoNT/A to the lateral lower orbicularis oculi muscle proximal to the first eye of the subject,
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a BoNT/A light-chain and translocation domain (H_{N}), and a BoNT/B receptor binding domain (H_{C} domain).

19. A modified botulinum neurotoxin A (BoNT/A) for use in a method of treating typical hemifacial spasm, wherein the modified BoNT/A is administered by intramuscular injection at a plurality of sites of the face of the subject, the method comprising:
a) administering a unit dose of the modified BoNT/A to the lateral upper orbicularis oculi muscle proximal to an eye affected by hemifacial spasm;
b) administering a unit dose of the modified BoNT/A to the medial upper orbicularis oculi muscle proximal to an eye affected by hemifacial spasm;
c) administering a unit dose of the modified BoNT/A to the lateral lower orbicularis oculi muscle proximal to an eye affected by hemifacial spasm; and
d) administering one or more unit dose of the modified BoNT/A to one or more further muscles affected by said hemifacial spasm in accordance with the following dosage regimen:
(i) one unit dose to an orbicularis oris upper muscle;
(ii) one unit dose to an orbicularis oris lower muscle;
(iii) one unit dose to a zygomaticus major muscle;
(iv) one unit dose to a zygomaticus minor muscle;
(v) up to five unit doses (preferably one unit dose) to a frontalis muscle;
(vi) one unit dose to a mentalis muscle;
(vii) one unit dose to a platysma muscle;
(viii) up to two unit doses (preferably one unit dose) to a corrugator muscle;
(ix) one unit dose to a buccinator muscle;
(x) up to two unit doses (preferably one unit dose) to a masseter muscle;
(xi) one unit dose to a procerus muscle;
(xii) one unit dose to a nasalis muscle; and/or
(xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

20. A method of treating typical hemifacial spasm, the method comprising administering a modified BoNT/A by intramuscular injection at a plurality of sites of the face of the subject, the method comprising:
a) administering a unit dose of the modified BoNT/A to the lateral upper orbicularis oculi muscle proximal to an eye affected by hemifacial spasm;
b) administering a unit dose of the modified BoNT/A to the medial upper orbicularis oculi muscle proximal to an eye affected by hemifacial spasm;
c) administering a unit dose of the modified BoNT/A to the lateral lower orbicularis oculi muscle proximal to an eye affected by hemifacial spasm; and
d) administering one or more unit dose of the modified BoNT/A to one or more further muscles affected by said hemifacial spasm in accordance with the following dosage regimen:
(i) one unit dose to an orbicularis oris upper muscle;
(ii) one unit dose to an orbicularis oris lower muscle;
(iii) one unit dose to a zygomaticus major muscle;
(iv) one unit dose to a zygomaticus minor muscle;
(v) up to five unit doses (preferably one unit dose) to a frontalis muscle;
(vi) one unit dose to a mentalis muscle;
(vii) one unit dose to a platysma muscle;
(viii) up to two unit doses (preferably one unit dose) to a corrugator muscle;
(ix) one unit dose to a buccinator muscle;
(x) up to two unit doses (preferably one unit dose) to a masseter muscle;
(xi) one unit dose to a procerus muscle;
(xii) one unit dose to a nasalis muscle; and/or
(xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

21. Use of a modified botulinum neurotoxin A (BoNT/A) in the manufacture of a medicament for treating typical hemifacial spasm, wherein the modified BoNT/A is administered by intramuscular injection at a plurality of sites of the face of the subject, the treatment comprising:
a) administering a unit dose of the modified BoNT/A to the lateral upper orbicularis oculi muscle proximal to an eye affected by hemifacial spasm;
b) administering a unit dose of the modified BoNT/A to the medial upper orbicularis oculi muscle proximal to an eye affected by hemifacial spasm;
c) administering a unit dose of the modified BoNT/A to the lateral lower orbicularis oculi muscle proximal to an eye affected by hemifacial spasm; and
d) administering one or more unit dose of the modified BoNT/A to one or more further muscles affected by said hemifacial spasm in accordance with the following dosage regimen:
(i) one unit dose to an orbicularis oris upper muscle;
(ii) one unit dose to an orbicularis oris lower muscle;
(iii) one unit dose to a zygomaticus major muscle;
(iv) one unit dose to a zygomaticus minor muscle;
(v) up to five unit doses (preferably one unit dose) to a frontalis muscle;
(vi) one unit dose to a mentalis muscle;
(vii) one unit dose to a platysma muscle;
(viii) up to two unit doses (preferably one unit dose) to a corrugator muscle;
(ix) one unit dose to a buccinator muscle;
(x) up to two unit doses (preferably one unit dose) to a masseter muscle;
(xi) one unit dose to a procerus muscle;
(xii) one unit dose to a nasalis muscle; and/or
(xiii) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

22. A modified botulinum neurotoxin A (BoNT/A) for use in treating atypical hemifacial spasm, wherein the modified BoNT/A is administered by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
(i) one unit dose to a zygomaticus major muscle;
(ii) one unit dose to a zygomaticus minor muscle;
(iii) up to five unit doses (preferably one unit dose) to a frontalis muscle;
(iv) one unit dose to a mentalis muscle;
(v) one unit dose to a platysma muscle;
(vi) up to two unit doses (preferably one unit dose) to a corrugator muscle;
(vii) one unit dose to a buccinator muscle;
(viii) up to two unit doses (preferably one unit dose) to a masseter muscle;
(ix) one unit dose to a procerus muscle;
(x) one unit dose to a nasalis muscle;
(xi) one unit dose to a lateral upper orbicularis oculi muscle;
(xii) one unit dose to a medial upper orbicularis oculi muscle;
(xiii) one unit dose to a lateral lower orbicularis oculi muscle; and/or
(xiv) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

23. A method of treating atypical hemifacial spasm, the method comprising administering a modified BoNT/A by intramuscular injection at a plurality of sites of the face of the subject, the method comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
(i) one unit dose to a zygomaticus major muscle;
(ii) one unit dose to a zygomaticus minor muscle;
(iii) up to five unit doses (preferably one unit dose) to a frontalis muscle;
(iv) one unit dose to a mentalis muscle;
(v) one unit dose to a platysma muscle;
(vi) up to two unit doses (preferably one unit dose) to a corrugator muscle;
(vii) one unit dose to a buccinator muscle;
(viii) up to two unit doses (preferably one unit dose) to a masseter muscle;
(ix) one unit dose to a procerus muscle;
(x) one unit dose to a nasalis muscle;
(xi) one unit dose to a lateral upper orbicularis oculi muscle;
(xii) one unit dose to a medial upper orbicularis oculi muscle;
(xiii) one unit dose to a lateral lower orbicularis oculi muscle; and/or
(xiv) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

24. Use of a modified botulinum neurotoxin A (BoNT/A) in the manufacture of a medicament for treating atypical hemifacial spasm, wherein the modified BoNT/A is administered by intramuscular injection at a plurality of sites of the face of the subject, the treatment comprising:
a) administering a unit dose of the modified BoNT/A to an orbicularis oris muscle affected by hemifacial spasm (e.g. administering one unit dose of the modified BoNT/A to an orbicularis oris upper muscle and/or one unit dose to an orbicularis oris lower muscle affected by hemifacial spasm; preferably administering one unit dose of the modified BoNT/A to an orbicularis oris upper muscle and one unit dose to an orbicularis oris lower muscle affected by said hemifacial spasm); and
b) optionally administering one or more unit dose of the modified BoNT/A to one or more further muscles affected by said hemifacial spasm in accordance with the following dosage regimen:
(i) one unit dose to a zygomaticus major muscle;
(ii) one unit dose to a zygomaticus minor muscle;
(iii) up to five unit doses (preferably one unit dose) to a frontalis muscle;
(iv) one unit dose to a mentalis muscle;
(v) one unit dose to a platysma muscle;
(vi) up to two unit doses (preferably one unit dose) to a corrugator muscle;
(vii) one unit dose to a buccinator muscle;
(viii) up to two unit doses (preferably one unit dose) to a masseter muscle;
(ix) one unit dose to a procerus muscle;
(x) one unit dose to a nasalis muscle;
(xi) one unit dose to a lateral upper orbicularis oculi muscle;
(xii) one unit dose to a medial upper orbicularis oculi muscle;
(xiii) one unit dose to a lateral lower orbicularis oculi muscle; and/or
(xiv) one unit dose to a levator palpebrae superiori muscle;
wherein the unit dose of the modified BoNT/A is at least 240 pg (preferably 240 pg to 8,000 pg) of modified BoNT/A,
wherein the total dose administered during the treatment is greater than 24,000 pg and up to 82,500 pg (e.g. up to 75,000 pg) of the modified BoNT/A, and
wherein the modified BoNT/A comprises a botulinum neurotoxin A (BoNT/A) light-chain and translocation domain (H_{N}), and a BoNT/B receptor binding domain (H_{C} domain).

25. The modified BoNT/A for use, the method or the use according to any one of the preceding claims, further comprising administering one or more unit dose of the modified BoNT/A to one or more further muscles affected by said blepharospasm in accordance with the following dosage regimen:
(i) one unit dose to an orbicularis oris upper muscle;
(ii) one unit dose to an orbicularis oris lower muscle;
(iii) one unit dose to a zygomaticus major muscle;
(iv) one unit dose to a zygomaticus minor muscle;
(v) up to five unit doses (preferably one unit dose) to a frontalis muscle;
(vi) one unit dose to a mentalis muscle;
(vii) one unit dose to a platysma muscle;
(viii) up to two unit doses (preferably one unit dose) to a corrugator muscle;
(ix) one unit dose to a buccinator muscle;
(x) up to two unit doses (preferably one unit dose) to a masseter muscle;
(xi) one unit dose to a procerus muscle;
(xii) one unit dose to a nasalis muscle; and/or
(xiii) one unit dose to a levator palpebrae superiori muscle.

26. The modified BoNT/A for use, the method or the use according to any one of the preceding claims, further comprising administering one or more unit dose of the modified BoNT/A to one or more further muscles affected by said blepharospasm in accordance with the following dosage regimen:
(i) one unit dose to a levator palpebrae superiori muscle.

27. The modified BoNT/A for use, the method or the use according to any one of the preceding claims, wherein the unit dose of the modified BoNT/A is 240 pg to 5,500 pg of modified BoNT/A.

28. The modified BoNT/A for use, the method or the use according to any one of the preceding claims, wherein the unit dose of the modified BoNT/A is 240 pg to 5,000 pg of modified BoNT/A.

29. The modified BoNT/A for use, the method or the use according to any one of the preceding claims, wherein the unit dose of the modified BoNT/A is 240 pg to 2,400 pg of modified BoNT/A.

30. The modified BoNT/A for use, the method or the use according to any one of the preceding claims, wherein the unit dose of the modified BoNT/A is 240 pg to 2,000 pg of modified BoNT/A.

31. The modified BoNT/A for use, the method or the use according to any one of the preceding claims, wherein the unit dose (e.g. the lower limit of the single unit dose) is at least 500 pg of modified BoNT/A.

32. The modified BoNT/A for use, the method or the use according to any one of the preceding claims, wherein the unit dose (e.g. the lower limit of the single unit dose) is at least 1,000 pg of modified BoNT/A.

33. The modified BoNT/A for use, the method or the use according to any one of claims 1-28, wherein the unit dose of the modified BoNT/A is 2,000 pg to 4,500 pg of modified BoNT/A.

34. The modified BoNT/A for use, the method or the use according to any one of the preceding claims, wherein the total dose administered during the treatment is 25,000 pg to 50,000 pg of modified BoNT/A.

35. The modified BoNT/A for use, the method or the use according to any one of claims 1-28 or 33, wherein the unit dose of the modified BoNT/A is 2,000 to 4,500 pg of modified BoNT/A; and wherein the total dose administered during the treatment is 25,000 pg to 50,000 pg of modified BoNT/A.

36. The modified BoNT/A for use, the method or the use according to any one of the preceding claims, wherein the modified BoNT/A comprises a polypeptide sequence having at least 70% sequence identity to SEQ ID NO: 6.

37. The modified BoNT/A for use, the method or the use according to any one of the preceding claims, wherein the modified BoNT/A has a Safety Ratio of greater than 7, wherein the Safety Ratio is calculated as: dose of toxin required for −10% bodyweight change measured as pg/mouse divided by DAS ED50 measured as pg/mouse, wherein ED50=dose required to produce a DAS score of 2.

38. The modified BoNT/A for use, the method or the use according to any one of the preceding claims, wherein the method for treating blepharospasm further comprises:
administering a unit dose of the modified BoNT/A to the lateral upper orbicularis oculi muscle proximal to a second eye of the subject;
administering a unit dose of the modified BoNT/A to the medial upper orbicularis oculi muscle proximal to the second eye of the subject; and
administering a unit dose of the modified BoNT/A to the lateral lower orbicularis oculi muscle proximal to the second eye of the subject.

39. The modified BoNT/A for use, the method or the use according to any one of the preceding claims, wherein the method for treating blepharospasm further comprises administering:
a unit dose of the modified BoNT/A to the medial lower orbicularis oculi muscle proximal to the eye of the subject, wherein the total dose of modified BoNT/A administered during the treatment does not exceed that stated (e.g. in the preceding claim).

40. The modified BoNT/A for use, the method or the use according to any one of the preceding claims, wherein the method for treating blepharospasm further comprises administering:
at least a unit dose (e.g. two unit doses) of the modified BoNT/A to the frontalis muscle proximal to the eye of the subject, wherein the total dose of modified BoNT/A administered during the treatment does not exceed that stated (e.g. in the preceding claim).

41. The modified BoNT/A for use, the method or the use according to any one of the preceding claims, wherein the method for treating blepharospasm further comprises administering:
at least a unit dose (e.g. two unit doses) of the modified BoNT/A to the corrugator muscle proximal to the eye of the subject, wherein the total dose of modified BoNT/A administered during the treatment does not exceed that stated (e.g. in the preceding claim).

42. The modified BoNT/A for use, the method or the use according to any one of the preceding claims, wherein the method for treating blepharospasm further comprises administering:
a unit dose of the modified BoNT/A to the medial lower orbicularis oculi muscle proximal to the eye of the subject, wherein the total dose of modified BoNT/A administered during the treatment does not exceed that stated (e.g. in the preceding claim).

43. The modified BoNT/A for use, the method or the use according to any one of the preceding claims, wherein the modified BoNT/A is administered by way of a single unit dose per injection site.

44. A unit dosage form of modified BoNT/A, the unit dosage form comprising:
a) at least 10 Units (preferably 10 Units to 333 Units) of modified BoNT/A, wherein 1 Unit is an amount of the modified BoNT/A that corresponds to the calculated median lethal dose (LD₅₀) in mice; or
b) at least 240 pg (preferably 240 pg to 8,000 pg) of modified BoNT/A; and
c) optionally a pharmaceutically acceptable carrier, excipient, adjuvant, and/or salt,
wherein the modified BoNT/A comprises a BoNT/A light-chain and translocation domain, and a BoNT/B receptor binding domain (H_{C} domain).

45. A kit comprising:
a) the unit dosage form according to claim 44; and
b) instructions for use of the same in treating blepharospasm; and
c) optionally a diluent.

46. A kit comprising:
(a) the unit dosage form according to claim 44; and
(b) instructions for use of the same in treating typical hemifacial spasm; and
(c) optionally a diluent.

47. A kit comprising:
(a) the unit dosage form according to claim 44; and
(b) instructions for use of the same in treating atypical hemifacial spasm; and
(c) optionally a diluent.
